SEQUENCE LISTING (427 sequences)

SEQ ID NO: 1 - DNA, 21 bp, Artificial Sequence
  Description of Artificial Sequence: Synthetic oligonucleotide
accacagtcttggtggagccg

SEQ ID NO: 2 - DNA, 21 bp, Artificial Sequence
  Description of Artificial Sequence: Synthetic oligonucleotide
ccagagccgaagctagactgg

SEQ ID NO: 3 - DNA, 21 bp, Artificial Sequence
  Description of Artificial Sequence: Synthetic oligonucleotide
gatgcccaaaacaagctggct

SEQ ID NO: 4 - DNA, 21 bp, Artificial Sequence
  Description of Artificial Sequence: Synthetic oligonucleotide
gcagcctccagggaagccctc

SEQ ID NO: 5 - DNA, 21 bp, Artificial Sequence
  Description of Artificial Sequence: Synthetic oligonucleotide
gcagcctccagggaagccctc

SEQ ID NO: 6 - DNA, 21 bp, Artificial Sequence
  Description of Artificial Sequence: Synthetic oligonucleotide
tcagtcttcgtacgacgaagg

SEQ ID NO: 7 - DNA, 21 bp, Artificial Sequence
  Description of Artificial Sequence: Synthetic oligonucleotide
cgtacgacgaaggtcatcccc

SEQ ID NO: 8 - DNA, 21 bp, Artificial Sequence
  Description of Artificial Sequence: Synthetic oligonucleotide
atctgggcctgcagctcacgg

SEQ ID NO: 9 - DNA, 21 bp, Artificial Sequence
  Description of Artificial Sequence: Synthetic oligonucleotide
tcagctcttctgccttctccc

SEQ ID NO: 10 - DNA, 21 bp, Artificial Sequence
  Description of Artificial Sequence: Synthetic oligonucleotide
gccgggcccgctcgtgtaaga

SEQ ID NO: 11 - DNA, 21 bp, Artificial Sequence
  Description of Artificial Sequence: Synthetic oligonucleotide
gccacagccgaccaaaggtat

SEQ ID NO: 12 - DNA, 21 bp, Artificial Sequence
  Description of Artificial Sequence: Synthetic oligonucleotide
ccacagccgaccaaaggtata

SEQ ID NO: 13 - DNA, 21 bp, Artificial Sequence
  Description of Artificial Sequence: Synthetic oligonucleotide
aagtttgcgaccagcttccaa

SEQ ID NO: 14 - DNA, 21 bp, Artificial Sequence
  Description of Artificial Sequence: Synthetic oligonucleotide
caccgtacggggcagaaagcg

SEQ ID NO: 15 - DNA, 21 bp, Artificial Sequence
  Description of Artificial Sequence: Synthetic oligonucleotide
cttggccaggtttgcactgag

SEQ ID NO: 16 - DNA, 21 bp, Artificial Sequence
  Description of Artificial Sequence: Synthetic oligonucleotide
cattagccccccttggtatct

SEQ ID NO: 17 - DNA, 21 bp, Artificial Sequence
  Description of Artificial Sequence: Synthetic oligonucleotide
ggccctgaatgccagcgacaa

SEQ ID NO: 18 - DNA, 21 bp, Artificial Sequence
  Description of Artificial Sequence: Synthetic oligonucleotide
ggccctgaatgccagcgacaa

SEQ ID NO: 19 - DNA, 21 bp, Artificial Sequence
  Description of Artificial Sequence: Synthetic oligonucleotide
tcctgaaaaatcctgatgtca

SEQ ID NO: 20 - DNA, 21 bp, Artificial Sequence
  Description of Artificial Sequence: Synthetic oligonucleotide
cgtttcacctactgcttctga

SEQ ID NO: 21 - DNA, 21 bp, Artificial Sequence
  Description of Artificial Sequence: Synthetic oligonucleotide
tgctggtatcctgttgcgttt

SEQ ID NO: 22 - DNA, 21 bp, Artificial Sequence
  Description of Artificial Sequence: Synthetic oligonucleotide
ctcttttaatgaggaagagat

SEQ ID NO: 23 - DNA, 21 bp, Artificial Sequence
  Description of Artificial Sequence: Synthetic oligonucleotide
ataagcccgaagggatgggga

SEQ ID NO: 24 - DNA, 21 bp, Artificial Sequence
  Description of Artificial Sequence: Synthetic oligonucleotide
tgggaaatgcggcggggtgag

SEQ ID NO: 25 - DNA, 21 bp, Artificial Sequence
  Description of Artificial Sequence: Synthetic oligonucleotide
ccatggcccaggtcgaagtga

SEQ ID NO: 26 - DNA, 21 bp, Artificial Sequence
  Description of Artificial Sequence: Synthetic oligonucleotide
gaatttcaagttggtgggtgc

SEQ ID NO: 27 - DNA, 21 bp, Artificial Sequence
  Description of Artificial Sequence: Synthetic oligonucleotide
ttttcttttgctagatgtctc

SEQ ID NO: 28 - DNA, 21 bp, Artificial Sequence
  Description of Artificial Sequence: Synthetic oligonucleotide
cttgattctccttttctagag

SEQ ID NO: 29 - DNA, 21 bp, Artificial Sequence
  Description of Artificial Sequence: Synthetic oligonucleotide
ttctacgggtgtaagtagttc

SEQ ID NO: 30 - DNA, 21 bp, Artificial Sequence
  Description of Artificial Sequence: Synthetic oligonucleotide
cggatgctctccaagatcgcc

SEQ ID NO: 31 - DNA, 21 bp, Artificial Sequence
  Description of Artificial Sequence: Synthetic oligonucleotide
gcctcgagtggctgctttctc

SEQ ID NO: 32 - DNA, 21 bp, Artificial Sequence
  Description of Artificial Sequence: Synthetic oligonucleotide
attcaagcgagtagacaatgg

SEQ ID NO: 33 - DNA, 21 bp, Artificial Sequence
  Description of Artificial Sequence: Synthetic oligonucleotide
cggttcacgtgcaacccagac

SEQ ID NO: 34 - DNA, 21 bp, Artificial Sequence
  Description of Artificial Sequence: Synthetic oligonucleotide
tcgctgaaggactctctccct

SEQ ID NO: 35 - DNA, 21 bp, Artificial Sequence
  Description of Artificial Sequence: Synthetic oligonucleotide
actgatccgaagctcccgcag

SEQ ID NO: 36 - DNA, 21 bp, Artificial Sequence
  Description of Artificial Sequence: Synthetic oligonucleotide
tttctttgatgcagtagatcc

SEQ ID NO: 37 - DNA, 21 bp, Artificial Sequence
  Description of Artificial Sequence: Synthetic oligonucleotide
tgcagtagatccaactctcat

SEQ ID NO: 38 - DNA, 21 bp, Artificial Sequence
  Description of Artificial Sequence: Synthetic oligonucleotide
ccacacccccacccctcctcc

SEQ ID NO: 39 - DNA, 21 bp, Artificial Sequence
  Description of Artificial Sequence: Synthetic oligonucleotide
cctttaccggcctggatgtgg

SEQ ID NO: 40 - DNA, 21 bp, Artificial Sequence
  Description of Artificial Sequence: Synthetic oligonucleotide
ggaagatgtctgtggattttc

SEQ ID NO: 41 - DNA, 21 bp, Artificial Sequence
  Description of Artificial Sequence: Synthetic oligonucleotide
tgggcacgctgcccctccacg

SEQ ID NO: 42 - DNA, 21 bp, Artificial Sequence
  Description of Artificial Sequence: Synthetic oligonucleotide
ggaggagacagaggctgcccg

SEQ ID NO: 43 - DNA, 21 bp, Artificial Sequence
  Description of Artificial Sequence: Synthetic oligonucleotide
tcggccatcttccactgcgtc

SEQ ID NO: 44 - DNA, 21 bp, Artificial Sequence
  Description of Artificial Sequence: Synthetic oligonucleotide
cgcgctatccagcagacggct

SEQ ID NO: 45 - DNA, 21 bp, Artificial Sequence
  Description of Artificial Sequence: Synthetic oligonucleotide
ctatgtccgtccctctggcca

SEQ ID NO: 46 - DNA, 21 bp, Artificial Sequence
  Description of Artificial Sequence: Synthetic oligonucleotide
gtggcacaggaggaaaagggg

SEQ ID NO: 47 - DNA, 21 bp, Artificial Sequence
  Description of Artificial Sequence: Synthetic oligonucleotide
tcctcgttggggtcgcccccg

SEQ ID NO: 48 - DNA, 21 bp, Artificial Sequence
  Description of Artificial Sequence: Synthetic oligonucleotide
gggagcacggtggggccccca

SEQ ID NO: 49 - DNA, 21 bp, Artificial Sequence
  Description of Artificial Sequence: Synthetic oligonucleotide
gctcccatgaggacattctcc

SEQ ID NO: 50 - DNA, 21 bp, Artificial Sequence
  Description of Artificial Sequence: Synthetic oligonucleotide
aggatgtcgaagcccaggttt

SEQ ID NO: 51 - DNA, 21 bp, Artificial Sequence
  Description of Artificial Sequence: Synthetic oligonucleotide
ctgatgccctgctggcagccc

SEQ ID NO: 52 - DNA, 21 bp, Artificial Sequence
  Description of Artificial Sequence: Synthetic oligonucleotide
tctacctgcagcccggggact

SEQ ID NO: 53 - skipped

SEQ ID NO: 54 - DNA, 4 bp, Artificial Sequence
  Description of Artificial Sequence: Synthetic oligonucleotide
ggta

SEQ ID NO: 55 - DNA, 4 bp, Artificial Sequence
  Description of Artificial Sequence: Synthetic oligonucleotide
gaag

SEQ ID NO: 56 - DNA, 4 bp, Artificial Sequence
  Description of Artificial Sequence: Synthetic oligonucleotide
tgag

SEQ ID NO: 57 - DNA, 4 bp, Artificial Sequence
  Description of Artificial Sequence: Synthetic oligonucleotide
ctgt

SEQ ID NO: 58 - DNA, 4 bp, Artificial Sequence
  Description of Artificial Sequence: Synthetic oligonucleotide
ctgt

SEQ ID NO: 59 - DNA, 4 bp, Artificial Sequence
  Description of Artificial Sequence: Synthetic oligonucleotide
gtca

SEQ ID NO: 60 - DNA, 4 bp, Artificial Sequence
  Description of Artificial Sequence: Synthetic oligonucleotide
cgtg

SEQ ID NO: 61 - DNA, 4 bp, Artificial Sequence
  Description of Artificial Sequence: Synthetic oligonucleotide
gatc

SEQ ID NO: 62 - DNA, 4 bp, Artificial Sequence
  Description of Artificial Sequence: Synthetic oligonucleotide
cggg

SEQ ID NO: 63 - DNA, 4 bp, Artificial Sequence
  Description of Artificial Sequence: Synthetic oligonucleotide
agcg

SEQ ID NO: 64 - DNA, 4 bp, Artificial Sequence
  Description of Artificial Sequence: Synthetic oligonucleotide
taat

SEQ ID NO: 65 - DNA, 4 bp, Artificial Sequence
  Description of Artificial Sequence: Synthetic oligonucleotide
aata

SEQ ID NO: 66 - DNA, 4 bp, Artificial Sequence
  Description of Artificial Sequence: Synthetic oligonucleotide
aaca

SEQ ID NO: 67 - DNA, 4 bp, Artificial Sequence
  Description of Artificial Sequence: Synthetic oligonucleotide
gcac

SEQ ID NO: 68 - DNA, 4 bp, Artificial Sequence
  Description of Artificial Sequence: Synthetic oligonucleotide
ggcc

SEQ ID NO: 69 - DNA, 4 bp, Artificial Sequence
  Description of Artificial Sequence: Synthetic oligonucleotide
tggg

SEQ ID NO: 70 - DNA, 4 bp, Artificial Sequence
  Description of Artificial Sequence: Synthetic oligonucleotide
agcg

SEQ ID NO: 71 - DNA, 4 bp, Artificial Sequence
  Description of Artificial Sequence: Synthetic oligonucleotide
agcg

SEQ ID NO: 72 - DNA, 4 bp, Artificial Sequence
  Description of Artificial Sequence: Synthetic oligonucleotide
agtg

SEQ ID NO: 73 - DNA, 4 bp, Artificial Sequence
  Description of Artificial Sequence: Synthetic oligonucleotide
atgc

SEQ ID NO: 74 - DNA, 4 bp, Artificial Sequence
  Description of Artificial Sequence: Synthetic oligonucleotide
tact

SEQ ID NO: 75 - DNA, 4 bp, Artificial Sequence
  Description of Artificial Sequence: Synthetic oligonucleotide
tgaa

SEQ ID NO: 76 - DNA, 4 bp, Artificial Sequence
  Description of Artificial Sequence: Synthetic oligonucleotide
aacc

SEQ ID NO: 77 - DNA, 4 bp, Artificial Sequence
  Description of Artificial Sequence: Synthetic oligonucleotide
gcct

SEQ ID NO: 78 - DNA, 4 bp, Artificial Sequence
  Description of Artificial Sequence: Synthetic oligonucleotide
agga

SEQ ID NO: 79 - DNA, 4 bp, Artificial Sequence
  Description of Artificial Sequence: Synthetic oligonucleotide
cagg

SEQ ID NO: 80 - DNA, 4 bp, Artificial Sequence
  Description of Artificial Sequence: Synthetic oligonucleotide
cagt

SEQ ID NO: 81 - DNA, 4 bp, Artificial Sequence
  Description of Artificial Sequence: Synthetic oligonucleotide
gatt

SEQ ID NO: 82 - DNA, 4 bp, Artificial Sequence
  Description of Artificial Sequence: Synthetic oligonucleotide
ctag

SEQ ID NO: 83 - DNA, 4 bp, Artificial Sequence
  Description of Artificial Sequence: Synthetic oligonucleotide
cacg

SEQ ID NO: 84 - DNA, 4 bp, Artificial Sequence
  Description of Artificial Sequence: Synthetic oligonucleotide
cagt

SEQ ID NO: 85 - DNA, 4 bp, Artificial Sequence
  Description of Artificial Sequence: Synthetic oligonucleotide
gaaa

SEQ ID NO: 86 - DNA, 4 bp, Artificial Sequence
  Description of Artificial Sequence: Synthetic oligonucleotide
caca

SEQ ID NO: 87 - DNA, 4 bp, Artificial Sequence
  Description of Artificial Sequence: Synthetic oligonucleotide
tggt

SEQ ID NO: 88 - DNA, 4 bp, Artificial Sequence
  Description of Artificial Sequence: Synthetic oligonucleotide
gccg

SEQ ID NO: 89 - DNA, 4 bp, Artificial Sequence
  Description of Artificial Sequence: Synthetic oligonucleotide
caac

SEQ ID NO: 90 - DNA, 4 bp, Artificial Sequence
  Description of Artificial Sequence: Synthetic oligonucleotide
tgat

SEQ ID NO: 91 - DNA, 4 bp, Artificial Sequence
  Description of Artificial Sequence: Synthetic oligonucleotide
cact

SEQ ID NO: 92 - DNA, 4 bp, Artificial Sequence
  Description of Artificial Sequence: Synthetic oligonucleotide
ggct

SEQ ID NO: 93 - DNA, 4 bp, Artificial Sequence
  Description of Artificial Sequence: Synthetic oligonucleotide
ctgt

SEQ ID NO: 94 - DNA, 4 bp, Artificial Sequence
  Description of Artificial Sequence: Synthetic oligonucleotide
gccc

SEQ ID NO: 95 - DNA, 4 bp, Artificial Sequence
  Description of Artificial Sequence: Synthetic oligonucleotide
ggga

SEQ ID NO: 96 - DNA, 4 bp, Artificial Sequence
  Description of Artificial Sequence: Synthetic oligonucleotide
cttg

SEQ ID NO: 97 - DNA, 4 bp, Artificial Sequence
  Description of Artificial Sequence: Synthetic oligonucleotide
tgac

SEQ ID NO: 98 - DNA, 4 bp, Artificial Sequence
  Description of Artificial Sequence: Synthetic oligonucleotide
atga

SEQ ID NO: 99 - DNA, 4 bp, Artificial Sequence
  Description of Artificial Sequence: Synthetic oligonucleotide
gctg

SEQ ID NO: 100 - DNA, 4 bp, Artificial Sequence
  Description of Artificial Sequence: Synthetic oligonucleotide
gacc

SEQ ID NO: 101 - DNA, 4 bp, Artificial Sequence
  Description of Artificial Sequence: Synthetic oligonucleotide
acaa

SEQ ID NO: 102 - DNA, 4 bp, Artificial Sequence
  Description of Artificial Sequence: Synthetic oligonucleotide
cgca

SEQ ID NO: 103 - DNA, 4 bp, Artificial Sequence
  Description of Artificial Sequence: Synthetic oligonucleotide
tggg

SEQ ID NO: 104 - DNA, 4 bp, Artificial Sequence
  Description of Artificial Sequence: Synthetic oligonucleotide
catg

SEQ ID NO: 105 - DNA, 4 bp, Artificial Sequence
  Description of Artificial Sequence: Synthetic oligonucleotide
tacc

SEQ ID NO: 106 - DNA, 21 bp, Artificial Sequence
  Description of Artificial Sequence: Synthetic oligonucleotide
accacagtcttggtggagccg

SEQ ID NO: 107 - DNA, 21 bp, Artificial Sequence
  Description of Artificial Sequence: Synthetic oligonucleotide
cagagccgaagctagactgga

SEQ ID NO: 108 - DNA, 21 bp, Artificial Sequence
  Description of Artificial Sequence: Synthetic oligonucleotide
gatgcccaaaacaagctggct

SEQ ID NO: 109 - DNA, 21 bp, Artificial Sequence
  Description of Artificial Sequence: Synthetic oligonucleotide
ggcagcctccagggaagccct

SEQ ID NO: 110 - skipped

SEQ ID NO: 111 - DNA, 21 bp, Artificial Sequence
  Description of Artificial Sequence: Synthetic oligonucleotide
ggcagcctccagggaagccct

SEQ ID NO: 112 - DNA, 21 bp, Artificial Sequence
  Description of Artificial Sequence: Synthetic oligonucleotide
gtcttcgtacgacgaaggtca

SEQ ID NO: 113 - DNA, 21 bp, Artificial Sequence
  Description of Artificial Sequence: Synthetic oligonucleotide
cgtacgacgaaggtcatcccc

SEQ ID NO: 114 - DNA, 21 bp, Artificial Sequence
  Description of Artificial Sequence: Synthetic oligonucleotide
ctgggcctgcagctcacggat

SEQ ID NO: 115 - DNA, 21 bp, Artificial Sequence
  Description of Artificial Sequence: Synthetic oligonucleotide
atcagctcttctgccttctcc

SEQ ID NO: 116 - DNA, 21 bp, Artificial Sequence
  Description of Artificial Sequence: Synthetic oligonucleotide
gccgggcccgctcgtgtaaga

SEQ ID NO: 117 - DNA, 21 bp, Artificial Sequence
  Description of Artificial Sequence: Synthetic oligonucleotide
ccacagccgaccaaaggtata

SEQ ID NO: 118 - DNA, 21 bp, Artificial Sequence
  Description of Artificial Sequence: Synthetic oligonucleotide
ccacagccgaccaaaggtata

SEQ ID NO: 119 - DNA, 21 bp, Artificial Sequence
  Description of Artificial Sequence: Synthetic oligonucleotide
agtttgcgaccagcttccaaa

SEQ ID NO: 120 - DNA, 21 bp, Artificial Sequence
  Description of Artificial Sequence: Synthetic oligonucleotide
caccgtacggggcagaaagcg

SEQ ID NO: 121 - DNA, 21 bp, Artificial Sequence
  Description of Artificial Sequence: Synthetic oligonucleotide
cttggccaggtttgcactgag

SEQ ID NO: 122 - DNA, 21 bp, Artificial Sequence
  Description of Artificial Sequence: Synthetic oligonucleotide
cattagccccccttggtatct

SEQ ID NO: 123 - DNA, 21 bp, Artificial Sequence
  Description of Artificial Sequence: Synthetic oligonucleotide
ggccctgaatgccagcgacaa

SEQ ID NO: 124 - DNA, 21 bp, Artificial Sequence
  Description of Artificial Sequence: Synthetic oligonucleotide
ggccctgaatgccagcgacaa

SEQ ID NO: 125 - DNA, 21 bp, Artificial Sequence
  Description of Artificial Sequence: Synthetic oligonucleotide
tcctgaaaaatcctgatgtca

SEQ ID NO: 126 - DNA, 21 bp, Artificial Sequence
  Description of Artificial Sequence: Synthetic oligonucleotide
cgtttcacctactgcttctga

SEQ ID NO: 127 - DNA, 21 bp, Artificial Sequence
  Description of Artificial Sequence: Synthetic oligonucleotide
gtctgctggtatcctgttgcg

SEQ ID NO: 128 - DNA, 21 bp, Artificial Sequence
  Description of Artificial Sequence: Synthetic oligonucleotide
ctcttttaatgaggaagagat

SEQ ID NO: 129 - DNA, 21 bp, Artificial Sequence
  Description of Artificial Sequence: Synthetic oligonucleotide
agcccgaagggatggggaacc

SEQ ID NO: 130 - DNA, 21 bp, Artificial Sequence
  Description of Artificial Sequence: Synthetic oligonucleotide
ggaaatgcggcggggtgagcc

SEQ ID NO: 131 - DNA, 21 bp, Artificial Sequence
  Description of Artificial Sequence: Synthetic oligonucleotide
gaatttcaagttggtgggtgc

SEQ ID NO: 132 - DNA, 21 bp, Artificial Sequence
  Description of Artificial Sequence: Synthetic oligonucleotide
tgattttcttttgctagatgt

SEQ ID NO: 133 - DNA, 21 bp, Artificial Sequence
  Description of Artificial Sequence: Synthetic oligonucleotide
tgattctccttttctagagat

SEQ ID NO: 134 - DNA, 21 bp, Artificial Sequence
  Description of Artificial Sequence: Synthetic oligonucleotide
tttctacgggtgtaagtagtt

SEQ ID NO: 135 - DNA, 21 bp, Artificial Sequence
  Description of Artificial Sequence: Synthetic oligonucleotide
gtgcggatgctctccaagatc

SEQ ID NO: 136 - DNA, 21 bp, Artificial Sequence
  Description of Artificial Sequence: Synthetic oligonucleotide
aaggcctcgagtggctgcttt

SEQ ID NO: 137 - DNA, 21 bp, Artificial Sequence
  Description of Artificial Sequence: Synthetic oligonucleotide
tcaagcgagtagacaatggaa

SEQ ID NO: 138 - DNA, 21 bp, Artificial Sequence
  Description of Artificial Sequence: Synthetic oligonucleotide
cggttcacgtgcaacccagac

SEQ ID NO: 139 - DNA, 21 bp, Artificial Sequence
  Description of Artificial Sequence: Synthetic oligonucleotide
acctcgctgaaggactctctc

SEQ ID NO: 140 - DNA, 21 bp, Artificial Sequence
  Description of Artificial Sequence: Synthetic oligonucleotide
actgatccgaagctcccgcag

SEQ ID NO: 141 - DNA, 21 bp, Artificial Sequence
  Description of Artificial Sequence: Synthetic oligonucleotide
tttgatgcagtagatccaact

SEQ ID NO: 142 - DNA, 21 bp, Artificial Sequence
  Description of Artificial Sequence: Synthetic oligonucleotide
tgcagtagatccaactctcat

SEQ ID NO: 143 - DNA, 21 bp, Artificial Sequence
  Description of Artificial Sequence: Synthetic oligonucleotide
ttaccggcctggatgtgggct

SEQ ID NO: 144 - DNA, 21 bp, Artificial Sequence
  Description of Artificial Sequence: Synthetic oligonucleotide
ggaagatgtctgtggattttc

SEQ ID NO: 145 - DNA, 21 bp, Artificial Sequence
  Description of Artificial Sequence: Synthetic oligonucleotide
cctgggcacgctgcccctcca

SEQ ID NO: 146 - DNA, 21 bp, Artificial Sequence
  Description of Artificial Sequence: Synthetic oligonucleotide
ggaggagacagaggctgcccg

SEQ ID NO: 147 - DNA, 21 bp, Artificial Sequence
  Description of Artificial Sequence: Synthetic oligonucleotide
cggccatcttccactgcgtct

SEQ ID NO: 148 - DNA, 21 bp, Artificial Sequence
  Description of Artificial Sequence: Synthetic oligonucleotide
gcgcgctatccagcagacggc

SEQ ID NO: 149 - DNA, 21 bp, Artificial Sequence
  Description of Artificial Sequence: Synthetic oligonucleotide
tatgtccgtccctctggccat

SEQ ID NO: 150 - DNA, 21 bp, Artificial Sequence
  Description of Artificial Sequence: Synthetic oligonucleotide
tggcacaggaggaaaaggggc

SEQ ID NO: 151 - DNA, 21 bp, Artificial Sequence
  Description of Artificial Sequence: Synthetic oligonucleotide
cctcctcgttggggtcgcccc

SEQ ID NO: 152 - DNA, 21 bp, Artificial Sequence
  Description of Artificial Sequence: Synthetic oligonucleotide
gggagcacggtggggccccca

SEQ ID NO: 153 - DNA, 21 bp, Artificial Sequence
  Description of Artificial Sequence: Synthetic oligonucleotide
cgctcccatgaggacattctc

SEQ ID NO: 154 - DNA, 21 bp, Artificial Sequence
  Description of Artificial Sequence: Synthetic oligonucleotide
aggatgtcgaagcccaggttt

SEQ ID NO: 155 - DNA, 21 bp, Artificial Sequence
  Description of Artificial Sequence: Synthetic oligonucleotide
tgatgccctgctggcagccca

SEQ ID NO: 156 - DNA, 21 bp, Artificial Sequence
  Description of Artificial Sequence: Synthetic oligonucleotide
acctgcagcccggggactacc

SEQ ID NO: 157 - DNA, 20 bp, Artificial Sequence
  Description of Artificial Sequence: Synthetic oligonucleotide
cagccccagcagtagggcca

SEQ ID NO: 158 - DNA, 4 bp, Artificial Sequence
  Description of Artificial Sequence: Synthetic oligonucleotide
ggta

SEQ ID NO: 159 - DNA, 4 bp, Artificial Sequence
  Description of Artificial Sequence: Synthetic oligonucleotide
aagg

SEQ ID NO: 160 - DNA, 4 bp, Artificial Sequence
  Description of Artificial Sequence: Synthetic oligonucleotide
tgag

SEQ ID NO: 161 - DNA, 4 bp, Artificial Sequence
  Description of Artificial Sequence: Synthetic oligonucleotide
tctg

SEQ ID NO: 162 - DNA, 4 bp, Artificial Sequence
  Description of Artificial Sequence: Synthetic oligonucleotide
tctg

SEQ ID NO: 163 - DNA, 4 bp, Artificial Sequence
  Description of Artificial Sequence: Synthetic oligonucleotide
atcc

SEQ ID NO: 164 - DNA, 4 bp, Artificial Sequence
  Description of Artificial Sequence: Synthetic oligonucleotide
cgtg

SEQ ID NO: 165 - DNA, 4 bp, Artificial Sequence
  Description of Artificial Sequence: Synthetic oligonucleotide
tctc

SEQ ID NO: 166 - DNA, 4 bp, Artificial Sequence
  Description of Artificial Sequence: Synthetic oligonucleotide
ccgg

SEQ ID NO: 167 - DNA, 4 bp, Artificial Sequence
  Description of Artificial Sequence: Synthetic oligonucleotide
agcg

SEQ ID NO: 168 - DNA, 4 bp, Artificial Sequence
  Description of Artificial Sequence: Synthetic oligonucleotide
aata

SEQ ID NO: 169 - DNA, 4 bp, Artificial Sequence
  Description of Artificial Sequence: Synthetic oligonucleotide
aata

SEQ ID NO: 170 - DNA, 4 bp, Artificial Sequence
  Description of Artificial Sequence: Synthetic oligonucleotide
acaa

SEQ ID NO: 171 - DNA, 4 bp, Artificial Sequence
  Description of Artificial Sequence: Synthetic oligonucleotide
gcac

SEQ ID NO: 172 - DNA, 4 bp, Artificial Sequence
  Description of Artificial Sequence: Synthetic oligonucleotide
ggcc

SEQ ID NO: 173 - DNA, 4 bp, Artificial Sequence
  Description of Artificial Sequence: Synthetic oligonucleotide
tggg

SEQ ID NO: 174 - DNA, 4 bp, Artificial Sequence
  Description of Artificial Sequence: Synthetic oligonucleotide
agcg

SEQ ID NO: 175 - DNA, 4 bp, Artificial Sequence
  Description of Artificial Sequence: Synthetic oligonucleotide
agcg

SEQ ID NO: 176 - DNA, 4 bp, Artificial Sequence
  Description of Artificial Sequence: Synthetic oligonucleotide
agtg

SEQ ID NO: 177 - DNA, 4 bp, Artificial Sequence
  Description of Artificial Sequence: Synthetic oligonucleotide
atgc

SEQ ID NO: 178 - DNA, 4 bp, Artificial Sequence
  Description of Artificial Sequence: Synthetic oligonucleotide
gttt

SEQ ID NO: 179 - DNA, 4 bp, Artificial Sequence
  Description of Artificial Sequence: Synthetic oligonucleotide
tgaa

SEQ ID NO: 180 - DNA, 4 bp, Artificial Sequence
  Description of Artificial Sequence: Synthetic oligonucleotide
ctga

SEQ ID NO: 181 - DNA, 4 bp, Artificial Sequence
  Description of Artificial Sequence: Synthetic oligonucleotide
ctgg

SEQ ID NO: 182 - DNA, 4 bp, Artificial Sequence
  Description of Artificial Sequence: Synthetic oligonucleotide
cagg

SEQ ID NO: 183 - DNA, 4 bp, Artificial Sequence
  Description of Artificial Sequence: Synthetic oligonucleotide
tctc

SEQ ID NO: 184 - DNA, 4 bp, Artificial Sequence
  Description of Artificial Sequence: Synthetic oligonucleotide
ttgc

SEQ ID NO: 185 - DNA, 4 bp, Artificial Sequence
  Description of Artificial Sequence: Synthetic oligonucleotide
tcta

SEQ ID NO: 186 - DNA, 4 bp, Artificial Sequence
  Description of Artificial Sequence: Synthetic oligonucleotide
cgcc

SEQ ID NO: 187 - DNA, 4 bp, Artificial Sequence
  Description of Artificial Sequence: Synthetic oligonucleotide
tctc

SEQ ID NO: 188 - DNA, 4 bp, Artificial Sequence
  Description of Artificial Sequence: Synthetic oligonucleotide
aagg

SEQ ID NO: 189 - DNA, 4 bp, Artificial Sequence
  Description of Artificial Sequence: Synthetic oligonucleotide
caca

SEQ ID NO: 190 - DNA, 4 bp, Artificial Sequence
  Description of Artificial Sequence: Synthetic oligonucleotide
ccct

SEQ ID NO: 191 - DNA, 4 bp, Artificial Sequence
  Description of Artificial Sequence: Synthetic oligonucleotide
gccg

SEQ ID NO: 192 - DNA, 4 bp, Artificial Sequence
  Description of Artificial Sequence: Synthetic oligonucleotide
tctc

SEQ ID NO: 193 - DNA, 4 bp, Artificial Sequence
  Description of Artificial Sequence: Synthetic oligonucleotide
tgat

SEQ ID NO: 194 - DNA, 4 bp, Artificial Sequence
  Description of Artificial Sequence: Synthetic oligonucleotide
ttgg

SEQ ID NO: 195 - DNA, 4 bp, Artificial Sequence
  Description of Artificial Sequence: Synthetic oligonucleotide
ctgt

SEQ ID NO: 196 - DNA, 4 bp, Artificial Sequence
  Description of Artificial Sequence: Synthetic oligonucleotide
acgc

SEQ ID NO: 197 - DNA, 4 bp, Artificial Sequence
  Description of Artificial Sequence: Synthetic oligonucleotide
ggga

SEQ ID NO: 198 - DNA, 4 bp, Artificial Sequence
  Description of Artificial Sequence: Synthetic oligonucleotide
ttgc

SEQ ID NO: 199 - DNA, 4 bp, Artificial Sequence
  Description of Artificial Sequence: Synthetic oligonucleotide
ctga

SEQ ID NO: 200 - DNA, 4 bp, Artificial Sequence
  Description of Artificial Sequence: Synthetic oligonucleotide
tgag

SEQ ID NO: 201 - DNA, 4 bp, Artificial Sequence
  Description of Artificial Sequence: Synthetic oligonucleotide
ctga

SEQ ID NO: 202 - DNA, 4 bp, Artificial Sequence
  Description of Artificial Sequence: Synthetic oligonucleotide
ccga

SEQ ID NO: 203 - DNA, 4 bp, Artificial Sequence
  Description of Artificial Sequence: Synthetic oligonucleotide
acaa

SEQ ID NO: 204 - DNA, 4 bp, Artificial Sequence
  Description of Artificial Sequence: Synthetic oligonucleotide
ccgc

SEQ ID NO: 205 - DNA, 4 bp, Artificial Sequence
  Description of Artificial Sequence: Synthetic oligonucleotide
tggg

SEQ ID NO: 206 - DNA, 4 bp, Artificial Sequence
  Description of Artificial Sequence: Synthetic oligonucleotide
atgt

SEQ ID NO: 207 - DNA, 4 bp, Artificial Sequence
  Description of Artificial Sequence: Synthetic oligonucleotide
cgtg

SEQ ID NO: 208 - DNA, 3 bp, Artificial Sequence
  Description of Artificial Sequence: Synthetic oligonucleotide
ggg

SEQ ID NO: 209 - DNA, 151 bp, Artificial Sequence
  Description of Artificial Sequence: Synthetic polynucleotide
gcctgggaaggccccctcatcagcatgagataggcagccaatctcttatccactggagaggcaccatcgaagaaaacctgaagaagggcttcctggatctgtagccaaaagaggggactattgtactatcaccgtcctttggtcagaacac

SEQ ID NO: 210 - DNA, 151 bp, Artificial Sequence
  Description of Artificial Sequence: Synthetic polynucleotide
gcccaggggaagaaagatccatgtgttcataaagcccatctgaaaaatccattacctttttcatctcttcctcatcaaaagagaagtttgcagatccatttatctgttttattgaaaagttcaattttatttctcactttagcaattcaaa

SEQ ID NO: 211 - DNA, 151 bp, Artificial Sequence
  Description of Artificial Sequence: Synthetic polynucleotide
gcacagggaaggagtggggcaagggaggaggagaaaggggatggtgggaggcaggagtctaccttggctccagtcaggcccttctctccttgcttgcccggttttccttcaggaccctgaagagagaggagggaaaagagtgagaggaagg

SEQ ID NO: 212 - DNA, 151 bp, Artificial Sequence
  Description of Artificial Sequence: Synthetic polynucleotide
ttcctcaccttgggcttcctattcacccagaactcaacaattcctgagaccgactcccaagtcacacacaaatgctatggtgcaaatgtatcaggatgctggaaaactacttcagtcccacccacagataagctgtattctcctggcttgt

SEQ ID NO: 213 - DNA, 151 bp, Artificial Sequence
  Description of Artificial Sequence: Synthetic polynucleotide
tagacaagatctcagctaccatctgaaacagcacctcacctgtctctgaaaaagcaatggagaggctaaggaaggccaggaaacacagcaacagcttctccatgttccctccttgctgtctgtgtggccagaactttggttatcctcttgg

SEQ ID NO: 214 - DNA, 151 bp, Artificial Sequence
  Description of Artificial Sequence: Synthetic polynucleotide
atgtcgcagagatgaccctcccagccttcgttgcaaacacactgcccgggctcgaagcagatcccattgacgcaggcaggagagggcacgcaatggtcacacaggggaccctgccagccagggtggcacctgcgggaaggaaaagtgggca

SEQ ID NO: 215 - DNA, 151 bp, Artificial Sequence
  Description of Artificial Sequence: Synthetic polynucleotide
agtcatctgaataactttatcaactttttcatgggtgactttgatactgagtttgctttttacctcttttgcagagcaaacagaaatgaccatcgaaggcttgcagcccacagtggagtatgtggttagcgtctatgctcagaatcgaaac

SEQ ID NO: 216 - DNA, 151 bp, Artificial Sequence
  Description of Artificial Sequence: Synthetic polynucleotide
tatttcttttttgtaggtgaatttatccatgaaaaatttagccaaaaggacttaaacagtaagaccattctttacgtcataaatccatctcgggaagtaaattcggatgtcatggaatttcaaatcatggaccctacagggaactctgccc

SEQ ID NO: 217 - DNA, 151 bp, Artificial Sequence
  Description of Artificial Sequence: Synthetic polynucleotide
agttacatcaccacagaaagccttaccactactgctgtgagatcagaggcagcagaacgagcacccagctccgaggtgcctgtcccagactatacctccatccatataatacagtctccacagggcctcgtcctcaacgcgactgccttgc

SEQ ID NO: 218 - DNA, 151 bp, Artificial Sequence
  Description of Artificial Sequence: Synthetic polynucleotide
atttgtgttttttaaagtccttcatgttcccgaccttcaacacctaaaagtgattcagaattggtcagcaagtccatggataggacagtgcagaagaataacccagaaatgctctggctgtggggagaattgccacaagctgcaaaggtta

SEQ ID NO: 219 - DNA, 151 bp, Artificial Sequence
  Description of Artificial Sequence: Synthetic polynucleotide
taataaggcgcgcaggccacgcgagcggcgcggcggccgggcgcaccgggcagggcggccgtggccatggctttgcccggggcgctggcgggtctggccgcgggcttgcagggcgcgcgggccgccccgagcccggacaccgactcggaca

SEQ ID NO: 220 - DNA, 151 bp, Artificial Sequence
  Description of Artificial Sequence: Synthetic polynucleotide
aggtacctggagagctgcagcgaggctgccacactgaagaggaagtatgagctcccagccgacactcaggccctgtatgccagcgacaagcgcaggacgacaggaggtagtgtgtttgctttgagccgtggagcgtgtgaggttgtgtgtc

SEQ ID NO: 221 - DNA, 151 bp, Artificial Sequence
  Description of Artificial Sequence: Synthetic polynucleotide
ttaccaatttcccgttttcttttaaggactgtgtttactgtgaaaacaaggggaaaggcaacttttgtgcaccgtgtgaggaagatattcctgctctgggactcagcaaagccacagacaccaaagaggaagaaagtggacccccctgagt

SEQ ID NO: 222 - DNA, 151 bp, Artificial Sequence
  Description of Artificial Sequence: Synthetic polynucleotide
ctgccttgcctggacacctaccttcactgcacactccacccccaggccccgtgtgggatgcggagaatgtcctcacgggagcgcctattggggacctgcagggacccttgagtcccctggtgcagcaggaggccctcgcagcccctcagga

SEQ ID NO: 223 - DNA, 151 bp, Artificial Sequence
  Description of Artificial Sequence: Synthetic polynucleotide
gtggtcttccactgggacttcggggatggggccccagtgcaggcaacagcagagccctgggctacccacatctacgtgcagcccggggactaccgtgtacaggtgaacgcgtccaacctggtcagcttctttgtggcacaggctgcggtga

SEQ ID NO: 224 - DNA, 151 bp, Artificial Sequence
  Description of Artificial Sequence: Synthetic polynucleotide
gagcttatcctcatgggtctccggtgcttttccccaggctgtgagcccgggtcccctgccacagagaacgacggcatgatggccttggccctgcagcaggagctggggcaggaacgagtacccacacttgaggagagcctggaggaggagg

SEQ ID NO: 225 - DNA, 151 bp, Artificial Sequence
  Description of Artificial Sequence: Synthetic polynucleotide
cttcactttgttccccttcagtttctcggttcaagctactacttgcttcatctgaaagtttgtcaatgtcaacatcctccaaaacagctgctgaactacctgatgcctcctctttaactgtagccagcaagaaagatgagtctttcttctt

SEQ ID NO: 226 - DNA, 151 bp, Artificial Sequence
  Description of Artificial Sequence: Synthetic polynucleotide
aggcctggcccctgagtgaggcccaggtgcaggcctcagtggcgaaggtcctggctgagttgctggagcagaagaagaaaaaggctgcggatgctgccaaggagagcagcaagaaggcccgaggaggcctcaagcggaagctgtcgggaga

SEQ ID NO: 227 - DNA, 151 bp, Artificial Sequence
  Description of Artificial Sequence: Synthetic polynucleotide
ctaacatctacccaacagcaactcaccgatttgatccaactctctgtttcctcctccggaagccgggacaccgtatggggcagaaagcgcaccaacttctccttgatgatggaaggtgtcaagatgtcctccatctccaccaggctcgcga

SEQ ID NO: 228 - DNA, 151 bp, Artificial Sequence
  Description of Artificial Sequence: Synthetic polynucleotide
cagtttgtctttttgtcgttctgggcactctatctgtatgaccgagaactcgtttactcaaaggtcctagataacatctttccaaaatggctgaatcacgcagtggtgagtgatgggaaagagtggggaacacagaaatccaaaagtctct

SEQ ID NO: 229 - skipped

SEQ ID NO: 230 - DNA, 151 bp, Artificial Sequence
  Description of Artificial Sequence: Synthetic polynucleotide
ctgcagctcattcggatggtgccaggactacaggaggccgccgggagacaggggagtcccgacttggaaggtgaagagttgaaggcacatgggttacaggggagcctaggagaaagaaaatgttcagattaccctggtctcccaggtacaa

SEQ ID NO: 231-233 - skipped (3 entries)

SEQ ID NO: 234 - DNA, 151 bp, Artificial Sequence
  Description of Artificial Sequence: Synthetic polynucleotide
gtgttctgaccaaaggacggtgatagtacaatagtcccctcttttggctacagatccaggaagcccttcttcaggttttcttcgatggtgcctctccagtggataagagattggctgcctatctcatgctgatgagggggccttcccaggc

SEQ ID NO: 235 - DNA, 151 bp, Artificial Sequence
  Description of Artificial Sequence: Synthetic polynucleotide
tttgaattgctaaagtgagaaataaaattgaacttttcaataaaacagataaatggatctgcaaacttctcttttgatgaggaagagatgaaaaaggtaatggatttttcagatgggctttatgaacacatggatctttcttcccctgggc

SEQ ID NO: 236 - DNA, 151 bp, Artificial Sequence
  Description of Artificial Sequence: Synthetic polynucleotide
ccttcctctcactcttttccctcctctctcttcagggtcctgaaggaaaaccgggcaagcaaggagagaagggcctgactggagccaaggtagactcctgcctcccaccatcccctttctcctcctcccttgccccactccttccctgtgc

SEQ ID NO: 237 - DNA, 151 bp, Artificial Sequence
  Description of Artificial Sequence: Synthetic polynucleotide
acaagccaggagaatacagcttatctgtgggtgggactgaagtagttttccagcatcctgatacatttgcaccatagcatttgtgtgtgacttgggagtcggtctcaggaattgttgagttctgggtgaataggaagcccaaggtgaggaa

SEQ ID NO: 238 - DNA, 151 bp, Artificial Sequence
  Description of Artificial Sequence: Synthetic polynucleotide
ccaagaggataaccaaagttctggccacacagacagcaaggagggaacatggagaagctgttgctgtgtttcctggccttccttagcctctccattgctttttcagagacaggtgaggtgctgtttcagatggtagctgagatcttgtcta

SEQ ID NO: 239 - DNA, 151 bp, Artificial Sequence
  Description of Artificial Sequence: Synthetic polynucleotide
tgcccacttttccttcccgcaggtgccaccctggctggcagggtcccctgtgtgaccattgcgtgccctctcctgcctgcgtcaatgggatctgcttcgagcccgggcagtgtgtttgcaacgaaggctgggagggtcatctctgcgacat

SEQ ID NO: 240 - DNA, 151 bp, Artificial Sequence
  Description of Artificial Sequence: Synthetic polynucleotide
gtttcgattctgagcatagacgctaaccacatactccactgtgggctgcaagccttcgatggtcatttctgtttgctctgcaaaagaggtaaaaagcaaactcagtatcaaagtcacccatgaaaaagttgataaagttattcagatgact

SEQ ID NO: 241 - DNA, 151 bp, Artificial Sequence
  Description of Artificial Sequence: Synthetic polynucleotide
gggcagagttccctgtagggtccatgatttgaaattccatgacatccgaatttacttcccgagatggatttatgacgtaaagaatggtcttactgtttaagtccttttggctaaatttttcatggataaattcacctacaaaaaagaaata

SEQ ID NO: 242 - DNA, 151 bp, Artificial Sequence
  Description of Artificial Sequence: Synthetic polynucleotide
gcaaggcagtcgcgttgaggacgaggccctgtggagactgtattatatggatggaggtatagtctgggacaggcacctcggagctgggtgctcgttctgctgcctctgatctcacagcagtagtggtaaggctttctgtggtgatgtaact

SEQ ID NO: 243 - DNA, 151 bp, Artificial Sequence
  Description of Artificial Sequence: Synthetic polynucleotide
taacctttgcagcttgtggcaattctccccacagccagagcatttctgggttattcttctgcactgtcctatccatggacttgctgaccaattctgaatcacttttaggtgttgaaggtcgggaacatgaaggactttaaaaaacacaaat

SEQ ID NO: 244 - DNA, 151 bp, Artificial Sequence
  Description of Artificial Sequence: Synthetic polynucleotide
tgtccgagtcggtgtccgggctcggggcggcccgcgcgccctgcaagcccgcggccagacccgccagcgccccgggcaaagccatggccacggccgccctgcccggtgcgcccggccgccgcgccgctcgcgtggcctgcgcgccttatta

SEQ ID NO: 245 - DNA, 151 bp, Artificial Sequence
  Description of Artificial Sequence: Synthetic polynucleotide
gacacacaacctcacacgctccacggctcaaagcaaacacactacctcctgtcgtcctgcgcttgtcgctggcatacagggcctgagtgtcggctgggagctcatacttcctcttcagtgtggcagcctcgctgcagctctccaggtacct

SEQ ID NO: 246 - DNA, 151 bp, Artificial Sequence
  Description of Artificial Sequence: Synthetic polynucleotide
actcaggggggtccactttcttcctctttggtgtctgtggctttgctgagtcccagagcaggaatatcttcctcacacggtgcacaaaagttgcctttccccttgttttcacagtaaacacagtccttaaaagaaaacgggaaattggtaa

SEQ ID NO: 247 - DNA, 151 bp, Artificial Sequence
  Description of Artificial Sequence: Synthetic polynucleotide
tcctgaggggctgcgagggcctcctgctgcaccaggggactcaagggtccctgcaggtccccaataggcgctcccgtgaggacattctccgcatcccacacggggcctgggggtggagtgtgcagtgaaggtaggtgtccaggcaaggcag

SEQ ID NO: 248 - DNA, 151 bp, Artificial Sequence
  Description of Artificial Sequence: Synthetic polynucleotide
tcaccgcagcctgtgccacaaagaagctgaccaggttggacgcgttcacctgtacacggtagtccccgggctgcacgtagatgtgggtagcccagggctctgctgttgcctgcactggggccccatccccgaagtcccagtggaagaccac

SEQ ID NO: 249 - DNA, 151 bp, Artificial Sequence
  Description of Artificial Sequence: Synthetic polynucleotide
cctcctcctccaggctctcctcaagtgtgggtactcgttcctgccccagctcctgctgcagggccaaggccatcatgccgtcgttctctgtggcaggggacccgggctcacagcctggggaaaagcaccggagacccatgaggataagctc

SEQ ID NO: 250 - DNA, 151 bp, Artificial Sequence
  Description of Artificial Sequence: Synthetic polynucleotide
aagaagaaagactcatctttcttgctggctacagttaaagaggaggcatcaggtagttcagcagctgttttggaggatgttgacattgacaaactttcagatgaagcaagtagtagcttgaaccgagaaactgaaggggaacaaagtgaag

SEQ ID NO: 251 - DNA, 151 bp, Artificial Sequence
  Description of Artificial Sequence: Synthetic polynucleotide
tctcccgacagcttccgcttgaggcctcctcgggccttcttgctgctctccttggcagcatccgcagcctttttcttcttctgctccagcaactcagccaggaccttcgccactgaggcctgcacctgggcctcactcaggggccaggcct

SEQ ID NO: 252 - DNA, 151 bp, Artificial Sequence
  Description of Artificial Sequence: Synthetic polynucleotide
tcgcgagcctggtggagatggaggacatcttgacaccttccatcatcaaggagaagttggtgcgctttctgccccatacggtgtcccggcttccggaggaggaaacagagagttggatcaaatcggtgagttgctgttgggtagatgttag

SEQ ID NO: 253 - DNA, 151 bp, Artificial Sequence
  Description of Artificial Sequence: Synthetic polynucleotide
agagacttttggatttctgtgttccccactctttcccatcactcaccactgcgtgattcagccattttggaaagatgttatctaggacctttgagtaaacgagttctcggtcatacagatagagtgcccagaacgacaaaaagacaaactg

SEQ ID NO: 254 - skipped

SEQ ID NO: 255 - DNA, 151 bp, Artificial Sequence
  Description of Artificial Sequence: Synthetic polynucleotide
ttgtacctgggagaccagggtaatctgaacattttctttctcctaggctcccctgtaacccatgtgccttcaactcttcaccttccaagtcgggactcccctgtctcccggcggcctcctgtagtcctggcaccatccgaatgagctgcag

SEQ ID NO: 256-257 - skipped (2 entries)

SEQ ID NO: 258 - DNA, 60 bp, Artificial Sequence
  Description of Artificial Sequence: Synthetic oligonucleotide
cctcttttggctacagatccgatccaggaagcccttcttccttcttcaggctttcttcga

SEQ ID NO: 259 - DNA, 40 bp, Artificial Sequence
  Description of Artificial Sequence: Synthetic oligonucleotide
tgcaaacttctcttttaatgtaatgaggaagagatgaaaa

SEQ ID NO: 260 - DNA, 100 bp, Artificial Sequence
  Description of Artificial Sequence: Synthetic polynucleotide
gaaggaaaaccgggcaagcaaccgggcaagcaaggagagaccgggcaagcaaggagagaacaaggagagaagggccagacgaagggccagactggagcca

SEQ ID NO: 261 - DNA, 40 bp, Artificial Sequence
  Description of Artificial Sequence: Synthetic oligonucleotide
cattgcatttgtgtgtgactattgcatttgtgtgtgactt

SEQ ID NO: 262 - skipped

SEQ ID NO: 263 - DNA, 60 bp, Artificial Sequence
  Description of Artificial Sequence: Synthetic oligonucleotide
gaccattgcgtgccctctccccctctcctggctgcgtcaacctctcctggctgcgtcaat

SEQ ID NO: 264 - DNA, 40 bp, Artificial Sequence
  Description of Artificial Sequence: Synthetic oligonucleotide
tgtgggctgcaagccttcgattctgtttgatctgcaaaag

SEQ ID NO: 265 - DNA, 40 bp, Artificial Sequence
  Description of Artificial Sequence: Synthetic oligonucleotide
tccgaatttacttcccgagatggatttatgatgtaaagaa

SEQ ID NO: 266 - skipped

SEQ ID NO: 267 - DNA, 120 bp, Artificial Sequence
  Description of Artificial Sequence: Synthetic polynucleotide
atatggatggaggtatagtctatggatggaggtatagtctatggaggtatagtctgggacatagtctgggacaggcatcttgggacaggcatctcggagcgggacaggcatctcggagct

SEQ ID NO: 268 - DNA, 20 bp, Artificial Sequence
  Description of Artificial Sequence: Synthetic oligonucleotide
cttctgcactgtcctatcca

SEQ ID NO: 269 - DNA, 60 bp, Artificial Sequence
  Description of Artificial Sequence: Synthetic oligonucleotide
ggccagacccgccagcgccccagcgccccggccaaagccacccggccaaagccatggcca

SEQ ID NO: 270 - DNA, 120 bp, Artificial Sequence
  Description of Artificial Sequence: Synthetic polynucleotide
tgtcgtcctgcgcttgtcgctgcgcttgtcgctggcattcgcgcttgtcgctggcattcaggcattcagggcctgagtgtttcagggcctgagtgtcggctcagggcctgagtgtcggct

SEQ ID NO: 271 - DNA, 40 bp, Artificial Sequence
  Description of Artificial Sequence: Synthetic oligonucleotide
gctttgctgagtcccagagcgcaggaatatcttcctcata

SEQ ID NO: 272 - DNA, 40 bp, Artificial Sequence
  Description of Artificial Sequence: Synthetic oligonucleotide
ggtccctgcaggtccccaatccccaataggcgctcccatg

SEQ ID NO: 273 - DNA, 140 bp, Artificial Sequence
  Description of Artificial Sequence: Synthetic polynucleotide
cacctgtacacggtagtcccacctgtacacggtagtcccccacggtagtccccgggctgcccccgggctgcaggtagatgcccgggctgcaggtagatgtcaggtagatgtgggtagcccaggtagatgtgggtagccca

SEQ ID NO: 274 - DNA, 120 bp, Artificial Sequence
  Description of Artificial Sequence: Synthetic polynucleotide
cctgccccagctcctgctgcctgccccagctcctgctgcacagctcctgctgcagggccacatcacgccgtcgttctctgacgccgtcgttctctgtggccgccgtcgttctctgtggca

SEQ ID NO: 275 - DNA, 20 bp, Artificial Sequence
  Description of Artificial Sequence: Synthetic oligonucleotide
aggtagttcagcagctgttt

SEQ ID NO: 276 - DNA, 80 bp, Artificial Sequence
  Description of Artificial Sequence: Synthetic oligonucleotide
ttccatcatcaaggagaagtggtgcgctttctgccccgtattctgccccgtacggtgtccccgtacggtgtcccggcttc

SEQ ID NO: 277 - DNA, 40 bp, Artificial Sequence
  Description of Artificial Sequence: Synthetic oligonucleotide
actgcgtgattcagccatttattttggaaagacgttatct

SEQ ID NO: 278 - skipped

SEQ ID NO: 279 - DNA, 40 bp, Artificial Sequence
  Description of Artificial Sequence: Synthetic oligonucleotide
caactattcaccttccaagtaactattcaccttccaagtc

SEQ ID NO: 280-281 - skipped (2 entries)

SEQ ID NO: 282 - DNA, 80 bp, Artificial Sequence
  Description of Artificial Sequence: Synthetic oligonucleotide
caatctcttatccactggagcatcgaagaaagcctgaagaatcgaagaaagcctgaagaaaagcctgaagaagggcttcc

SEQ ID NO: 283 - DNA, 40 bp, Artificial Sequence
  Description of Artificial Sequence: Synthetic oligonucleotide
tgggaggcaggagtctacctagtctaccttggctccagtc

SEQ ID NO: 284 - DNA, 40 bp, Artificial Sequence
  Description of Artificial Sequence: Synthetic oligonucleotide
caagtcacacacaaatgcaatgcaatggtgcaaatgtatc

SEQ ID NO: 285 - DNA, 80 bp, Artificial Sequence
  Description of Artificial Sequence: Synthetic oligonucleotide
tctctgaaaaagcaatggagaaaaagcaatggagaggctaagcaatggagaggctaaggatggagaggctaaggaaggtc

SEQ ID NO: 286 - DNA, 80 bp, Artificial Sequence
  Description of Artificial Sequence: Synthetic oligonucleotide
cagatcccattgacgcagcccccattgacgcagccaggagccattgacgcagccaggagaagccaggagagggcacgcaa

SEQ ID NO: 287 - DNA, 20 bp, Artificial Sequence
  Description of Artificial Sequence: Synthetic oligonucleotide
caaacagaaatgaccatcga

SEQ ID NO: 288 - DNA, 40 bp, Artificial Sequence
  Description of Artificial Sequence: Synthetic oligonucleotide
tttacatcataaatccatctttacatcataaatccatctc

SEQ ID NO: 289 - DNA, 60 bp, Artificial Sequence
  Description of Artificial Sequence: Synthetic oligonucleotide
acctaaaagtgattcagaatagaattggtcagcaagtccgtggtcagcaagtccgtggat

SEQ ID NO: 290 - DNA, 200 bp, Artificial Sequence
  Description of Artificial Sequence: Synthetic polynucleotide
cgcaccgggcagggcggccggggcagggcggccgtggccaggcggccgtggccatggcttgccgtggccatggctttggcccgtggccatggctttggcccgtggccatggctttggccgcatggctttggccggggcgcggctttggccggggcgctgggctttggccggggcgctggcggccggggcgctggcgggtc

SEQ ID NO: 291 - DNA, 40 bp, Artificial Sequence
  Description of Artificial Sequence: Synthetic oligonucleotide
tgagctcccagccgacactctgaatgccagcgacaagcgc

SEQ ID NO: 292 - DNA, 40 bp, Artificial Sequence
  Description of Artificial Sequence: Synthetic oligonucleotide
caacttttgtgcaccgtatgtgaggaagatattcctgctc

SEQ ID NO: 293 - DNA, 120 bp, Artificial Sequence
  Description of Artificial Sequence: Synthetic polynucleotide
cccaggccccgtgtgggatgggatgcggagaatgtcctcagatgcggagaatgtcctcatgtcctcatgggagcgcctattcctcatgggagcgcctattcctcatgggagcgcctattg

SEQ ID NO: 294 - DNA, 80 bp, Artificial Sequence
  Description of Artificial Sequence: Synthetic oligonucleotide
gcaggcaacagcagagccctacccacatctacctgcagcccccacatctacctgcagcccccacatctacctgcagcccg

SEQ ID NO: 295 - DNA, 60 bp, Artificial Sequence
  Description of Artificial Sequence: Synthetic oligonucleotide
tcccctgccacagagaacgacacagagaacgacggcgtgagaacgacggcgtgatggcct

SEQ ID NO: 296 - DNA, 80 bp, Artificial Sequence
  Description of Artificial Sequence: Synthetic oligonucleotide
gaaggtcctggctgagttgcctgagttgctggagcagaaggctggagcagaagaggaaaagcagaagaggaaaaaggctg

SEQ ID NO: 297 - DNA, 100 bp, Artificial Sequence
  Description of Artificial Sequence: Synthetic polynucleotide
ctgtttcctcctccggaagctgtttcctcctccggaagccccggaagccgggacaccgtacggaagccgggacaccgtacggaagccgggacaccgtacg

SEQ ID NO: 298 - DNA, 40 bp, Artificial Sequence
  Description of Artificial Sequence: Synthetic oligonucleotide
ccgagaactcgtttactcaatagataacgtctttccaaaa

SEQ ID NO: 299 - skipped

SEQ ID NO: 300 - DNA, 100 bp, Artificial Sequence
  Description of Artificial Sequence: Synthetic polynucleotide
gagacaggggagtcccgactcaggggagtcccgacttggacttggaaggtgaatagttgaggtgaatagttgaaggcacagtgaatagttgaaggcacat

SEQ ID NO: 301 - DNA, 20 bp, Artificial Sequence
  Description of Artificial Sequence: Synthetic oligonucleotide
cctcttttggctacagatcc

SEQ ID NO: 302 - DNA, 20 bp, Artificial Sequence
  Description of Artificial Sequence: Synthetic oligonucleotide
gatccaggaagcccttcttc

SEQ ID NO: 303 - DNA, 20 bp, Artificial Sequence
  Description of Artificial Sequence: Synthetic oligonucleotide
cttcttcaggctttcttcga

SEQ ID NO: 304 - DNA, 20 bp, Artificial Sequence
  Description of Artificial Sequence: Synthetic oligonucleotide
tgcaaacttctcttttaatg

SEQ ID NO: 305 - DNA, 20 bp, Artificial Sequence
  Description of Artificial Sequence: Synthetic oligonucleotide
taatgaggaagagatgaaaa

SEQ ID NO: 306 - DNA, 20 bp, Artificial Sequence
  Description of Artificial Sequence: Synthetic oligonucleotide
gaaggaaaaccgggcaagca

SEQ ID NO: 307 - DNA, 20 bp, Artificial Sequence
  Description of Artificial Sequence: Synthetic oligonucleotide
accgggcaagcaaggagaga

SEQ ID NO: 308 - DNA, 20 bp, Artificial Sequence
  Description of Artificial Sequence: Synthetic oligonucleotide
ccgggcaagcaaggagagaa

SEQ ID NO: 309 - DNA, 20 bp, Artificial Sequence
  Description of Artificial Sequence: Synthetic oligonucleotide
caaggagagaagggccagac

SEQ ID NO: 310 - DNA, 20 bp, Artificial Sequence
  Description of Artificial Sequence: Synthetic oligonucleotide
gaagggccagactggagcca

SEQ ID NO: 311 - DNA, 20 bp, Artificial Sequence
  Description of Artificial Sequence: Synthetic oligonucleotide
cattgcatttgtgtgtgact

SEQ ID NO: 312 - DNA, 20 bp, Artificial Sequence
  Description of Artificial Sequence: Synthetic oligonucleotide
attgcatttgtgtgtgactt

SEQ ID NO: 313 - DNA, 20 bp, Artificial Sequence
  Description of Artificial Sequence: Synthetic oligonucleotide
gaccattgcgtgccctctcc

SEQ ID NO: 314 - DNA, 20 bp, Artificial Sequence
  Description of Artificial Sequence: Synthetic oligonucleotide
ccctctcctggctgcgtcaa

SEQ ID NO: 315 - DNA, 20 bp, Artificial Sequence
  Description of Artificial Sequence: Synthetic oligonucleotide
cctctcctggctgcgtcaat

SEQ ID NO: 316 - DNA, 20 bp, Artificial Sequence
  Description of Artificial Sequence: Synthetic oligonucleotide
tgtgggctgcaagccttcga

SEQ ID NO: 317 - DNA, 20 bp, Artificial Sequence
  Description of Artificial Sequence: Synthetic oligonucleotide
ttctgtttgatctgcaaaag

SEQ ID NO: 318 - DNA, 20 bp, Artificial Sequence
  Description of Artificial Sequence: Synthetic oligonucleotide
tccgaatttacttcccgaga

SEQ ID NO: 319 - DNA, 20 bp, Artificial Sequence
  Description of Artificial Sequence: Synthetic oligonucleotide
tggatttatgatgtaaagaa

SEQ ID NO: 320 - DNA, 20 bp, Artificial Sequence
  Description of Artificial Sequence: Synthetic oligonucleotide
atatggatggaggtatagtc

SEQ ID NO: 321 - DNA, 20 bp, Artificial Sequence
  Description of Artificial Sequence: Synthetic oligonucleotide
tatggatggaggtatagtct

SEQ ID NO: 322 - DNA, 20 bp, Artificial Sequence
  Description of Artificial Sequence: Synthetic oligonucleotide
atggaggtatagtctgggac

SEQ ID NO: 323 - DNA, 20 bp, Artificial Sequence
  Description of Artificial Sequence: Synthetic oligonucleotide
atagtctgggacaggcatct

SEQ ID NO: 324 - DNA, 20 bp, Artificial Sequence
  Description of Artificial Sequence: Synthetic oligonucleotide
tgggacaggcatctcggagc

SEQ ID NO: 325 - DNA, 20 bp, Artificial Sequence
  Description of Artificial Sequence: Synthetic oligonucleotide
gggacaggcatctcggagct

SEQ ID NO: 326 - DNA, 20 bp, Artificial Sequence
  Description of Artificial Sequence: Synthetic oligonucleotide
cttctgcactgtcctatcca

SEQ ID NO: 327 - DNA, 20 bp, Artificial Sequence
  Description of Artificial Sequence: Synthetic oligonucleotide
ggccagacccgccagcgccc

SEQ ID NO: 328 - DNA, 20 bp, Artificial Sequence
  Description of Artificial Sequence: Synthetic oligonucleotide
cagcgccccggccaaagcca

SEQ ID NO: 329 - DNA, 20 bp, Artificial Sequence
  Description of Artificial Sequence: Synthetic oligonucleotide
cccggccaaagccatggcca

SEQ ID NO: 330 - DNA, 20 bp, Artificial Sequence
  Description of Artificial Sequence: Synthetic oligonucleotide
tgtcgtcctgcgcttgtcgc

SEQ ID NO: 331 - DNA, 20 bp, Artificial Sequence
  Description of Artificial Sequence: Synthetic oligonucleotide
tgcgcttgtcgctggcattc

SEQ ID NO: 332 - DNA, 20 bp, Artificial Sequence
  Description of Artificial Sequence: Synthetic oligonucleotide
gcgcttgtcgctggcattca

SEQ ID NO: 333 - DNA, 20 bp, Artificial Sequence
  Description of Artificial Sequence: Synthetic oligonucleotide
ggcattcagggcctgagtgt

SEQ ID NO: 334 - DNA, 20 bp, Artificial Sequence
  Description of Artificial Sequence: Synthetic oligonucleotide
ttcagggcctgagtgtcggc

SEQ ID NO: 335 - DNA, 20 bp, Artificial Sequence
  Description of Artificial Sequence: Synthetic oligonucleotide
tcagggcctgagtgtcggct

SEQ ID NO: 336 - DNA, 20 bp, Artificial Sequence
  Description of Artificial Sequence: Synthetic oligonucleotide
gctttgctgagtcccagagc

SEQ ID NO: 337 - DNA, 20 bp, Artificial Sequence
  Description of Artificial Sequence: Synthetic oligonucleotide
gcaggaatatcttcctcata

SEQ ID NO: 338 - DNA, 20 bp, Artificial Sequence
  Description of Artificial Sequence: Synthetic oligonucleotide
ggtccctgcaggtccccaat

SEQ ID NO: 339 - DNA, 20 bp, Artificial Sequence
  Description of Artificial Sequence: Synthetic oligonucleotide
ccccaataggcgctcccatg

SEQ ID NO: 340 - DNA, 20 bp, Artificial Sequence
  Description of Artificial Sequence: Synthetic oligonucleotide
cacctgtacacggtagtccc

SEQ ID NO: 341 - DNA, 20 bp, Artificial Sequence
  Description of Artificial Sequence: Synthetic oligonucleotide
acctgtacacggtagtcccc

SEQ ID NO: 342 - DNA, 20 bp, Artificial Sequence
  Description of Artificial Sequence: Synthetic oligonucleotide
cacggtagtccccgggctgc

SEQ ID NO: 343 - DNA, 20 bp, Artificial Sequence
  Description of Artificial Sequence: Synthetic oligonucleotide
ccccgggctgcaggtagatg

SEQ ID NO: 344 - DNA, 20 bp, Artificial Sequence
  Description of Artificial Sequence: Synthetic oligonucleotide
cccgggctgcaggtagatgt

SEQ ID NO: 345 - DNA, 20 bp, Artificial Sequence
  Description of Artificial Sequence: Synthetic oligonucleotide
caggtagatgtgggtagccc

SEQ ID NO: 346 - DNA, 20 bp, Artificial Sequence
  Description of Artificial Sequence: Synthetic oligonucleotide
aggtagatgtgggtagccca

SEQ ID NO: 347 - DNA, 20 bp, Artificial Sequence
  Description of Artificial Sequence: Synthetic oligonucleotide
cctgccccagctcctgctgc

SEQ ID NO: 348 - DNA, 20 bp, Artificial Sequence
  Description of Artificial Sequence: Synthetic oligonucleotide
ctgccccagctcctgctgca

SEQ ID NO: 349 - DNA, 20 bp, Artificial Sequence
  Description of Artificial Sequence: Synthetic oligonucleotide
cagctcctgctgcagggcca

SEQ ID NO: 350 - DNA, 20 bp, Artificial Sequence
  Description of Artificial Sequence: Synthetic oligonucleotide
catcacgccgtcgttctctg

SEQ ID NO: 351 - DNA, 20 bp, Artificial Sequence
  Description of Artificial Sequence: Synthetic oligonucleotide
acgccgtcgttctctgtggc

SEQ ID NO: 352 - DNA, 20 bp, Artificial Sequence
  Description of Artificial Sequence: Synthetic oligonucleotide
cgccgtcgttctctgtggca

SEQ ID NO: 353 - DNA, 20 bp, Artificial Sequence
  Description of Artificial Sequence: Synthetic oligonucleotide
aggtagttcagcagctgttt

SEQ ID NO: 354 - DNA, 20 bp, Artificial Sequence
  Description of Artificial Sequence: Synthetic oligonucleotide
ttccatcatcaaggagaagt

SEQ ID NO: 355 - DNA, 20 bp, Artificial Sequence
  Description of Artificial Sequence: Synthetic oligonucleotide
ggtgcgctttctgccccgta

SEQ ID NO: 356 - DNA, 20 bp, Artificial Sequence
  Description of Artificial Sequence: Synthetic oligonucleotide
ttctgccccgtacggtgtcc

SEQ ID NO: 357 - DNA, 20 bp, Artificial Sequence
  Description of Artificial Sequence: Synthetic oligonucleotide
ccgtacggtgtcccggcttc

SEQ ID NO: 358 - DNA, 20 bp, Artificial Sequence
  Description of Artificial Sequence: Synthetic oligonucleotide
actgcgtgattcagccattt

SEQ ID NO: 359 - DNA, 20 bp, Artificial Sequence
  Description of Artificial Sequence: Synthetic oligonucleotide
attttggaaagacgttatct

SEQ ID NO: 360 - DNA, 20 bp, Artificial Sequence
  Description of Artificial Sequence: Synthetic oligonucleotide
caactattcaccttccaagt

SEQ ID NO: 361 - DNA, 20 bp, Artificial Sequence
  Description of Artificial Sequence: Synthetic oligonucleotide
aactattcaccttccaagtc

SEQ ID NO: 362 - DNA, 20 bp, Artificial Sequence
  Description of Artificial Sequence: Synthetic oligonucleotide
caatctcttatccactggag

SEQ ID NO: 363 - DNA, 20 bp, Artificial Sequence
  Description of Artificial Sequence: Synthetic oligonucleotide
catcgaagaaagcctgaaga

SEQ ID NO: 364 - DNA, 20 bp, Artificial Sequence
  Description of Artificial Sequence: Synthetic oligonucleotide
atcgaagaaagcctgaagaa

SEQ ID NO: 365 - DNA, 20 bp, Artificial Sequence
  Description of Artificial Sequence: Synthetic oligonucleotide
aagcctgaagaagggcttcc

SEQ ID NO: 366 - DNA, 20 bp, Artificial Sequence
  Description of Artificial Sequence: Synthetic oligonucleotide
tgggaggcaggagtctacct

SEQ ID NO: 367 - DNA, 20 bp, Artificial Sequence
  Description of Artificial Sequence: Synthetic oligonucleotide
agtctaccttggctccagtc

SEQ ID NO: 368 - DNA, 20 bp, Artificial Sequence
  Description of Artificial Sequence: Synthetic oligonucleotide
caagtcacacacaaatgcaa

SEQ ID NO: 369 - DNA, 20 bp, Artificial Sequence
  Description of Artificial Sequence: Synthetic oligonucleotide
tgcaatggtgcaaatgtatc

SEQ ID NO: 370 - DNA, 20 bp, Artificial Sequence
  Description of Artificial Sequence: Synthetic oligonucleotide
tctctgaaaaagcaatggag

SEQ ID NO: 371 - DNA, 20 bp, Artificial Sequence
  Description of Artificial Sequence: Synthetic oligonucleotide
aaaaagcaatggagaggcta

SEQ ID NO: 372 - DNA, 20 bp, Artificial Sequence
  Description of Artificial Sequence: Synthetic oligonucleotide
agcaatggagaggctaagga

SEQ ID NO: 373 - DNA, 20 bp, Artificial Sequence
  Description of Artificial Sequence: Synthetic oligonucleotide
tggagaggctaaggaaggtc

SEQ ID NO: 374 - DNA, 20 bp, Artificial Sequence
  Description of Artificial Sequence: Synthetic oligonucleotide
cagatcccattgacgcagcc

SEQ ID NO: 375 - DNA, 20 bp, Artificial Sequence
  Description of Artificial Sequence: Synthetic oligonucleotide
cccattgacgcagccaggag

SEQ ID NO: 376 - DNA, 20 bp, Artificial Sequence
  Description of Artificial Sequence: Synthetic oligonucleotide
ccattgacgcagccaggaga

SEQ ID NO: 377 - DNA, 20 bp, Artificial Sequence
  Description of Artificial Sequence: Synthetic oligonucleotide
agccaggagagggcacgcaa

SEQ ID NO: 378 - DNA, 20 bp, Artificial Sequence
  Description of Artificial Sequence: Synthetic oligonucleotide
caaacagaaatgaccatcga

SEQ ID NO: 379 - DNA, 20 bp, Artificial Sequence
  Description of Artificial Sequence: Synthetic oligonucleotide
tttacatcataaatccatct

SEQ ID NO: 380 - DNA, 20 bp, Artificial Sequence
  Description of Artificial Sequence: Synthetic oligonucleotide
ttacatcataaatccatctc

SEQ ID NO: 381 - DNA, 20 bp, Artificial Sequence
  Description of Artificial Sequence: Synthetic oligonucleotide
acctaaaagtgattcagaat

SEQ ID NO: 382 - DNA, 20 bp, Artificial Sequence
  Description of Artificial Sequence: Synthetic oligonucleotide
agaattggtcagcaagtccg

SEQ ID NO: 383 - DNA, 20 bp, Artificial Sequence
  Description of Artificial Sequence: Synthetic oligonucleotide
tggtcagcaagtccgtggat

SEQ ID NO: 384 - DNA, 20 bp, Artificial Sequence
  Description of Artificial Sequence: Synthetic oligonucleotide
cgcaccgggcagggcggccg

SEQ ID NO: 385 - DNA, 20 bp, Artificial Sequence
  Description of Artificial Sequence: Synthetic oligonucleotide
gggcagggcggccgtggcca

SEQ ID NO: 386 - DNA, 20 bp, Artificial Sequence
  Description of Artificial Sequence: Synthetic oligonucleotide
ggcggccgtggccatggctt

SEQ ID NO: 387 - DNA, 20 bp, Artificial Sequence
  Description of Artificial Sequence: Synthetic oligonucleotide
gccgtggccatggctttggc

SEQ ID NO: 388 - DNA, 20 bp, Artificial Sequence
  Description of Artificial Sequence: Synthetic oligonucleotide
ccgtggccatggctttggcc

SEQ ID NO: 389 - DNA, 20 bp, Artificial Sequence
  Description of Artificial Sequence: Synthetic oligonucleotide
cgtggccatggctttggccg

SEQ ID NO: 390 - DNA, 20 bp, Artificial Sequence
  Description of Artificial Sequence: Synthetic oligonucleotide
catggctttggccggggcgc

SEQ ID NO: 391 - DNA, 20 bp, Artificial Sequence
  Description of Artificial Sequence: Synthetic oligonucleotide
ggctttggccggggcgctgg

SEQ ID NO: 392 - DNA, 20 bp, Artificial Sequence
  Description of Artificial Sequence: Synthetic oligonucleotide
gctttggccggggcgctggc

SEQ ID NO: 393 - DNA, 20 bp, Artificial Sequence
  Description of Artificial Sequence: Synthetic oligonucleotide
ggccggggcgctggcgggtc

SEQ ID NO: 394 - DNA, 20 bp, Artificial Sequence
  Description of Artificial Sequence: Synthetic oligonucleotide
tgagctcccagccgacactc

SEQ ID NO: 395 - DNA, 20 bp, Artificial Sequence
  Description of Artificial Sequence: Synthetic oligonucleotide
tgaatgccagcgacaagcgc

SEQ ID NO: 396 - DNA, 20 bp, Artificial Sequence
  Description of Artificial Sequence: Synthetic oligonucleotide
caacttttgtgcaccgtatg

SEQ ID NO: 397 - DNA, 20 bp, Artificial Sequence
  Description of Artificial Sequence: Synthetic oligonucleotide
tgaggaagatattcctgctc

SEQ ID NO: 398 - DNA, 20 bp, Artificial Sequence
  Description of Artificial Sequence: Synthetic oligonucleotide
cccaggccccgtgtgggatg

SEQ ID NO: 399 - DNA, 20 bp, Artificial Sequence
  Description of Artificial Sequence: Synthetic oligonucleotide
ggatgcggagaatgtcctca

SEQ ID NO: 400 - DNA, 20 bp, Artificial Sequence
  Description of Artificial Sequence: Synthetic oligonucleotide
gatgcggagaatgtcctcat

SEQ ID NO: 401 - DNA, 20 bp, Artificial Sequence
  Description of Artificial Sequence: Synthetic oligonucleotide
gtcctcatgggagcgcctat

SEQ ID NO: 402 - DNA, 20 bp, Artificial Sequence
  Description of Artificial Sequence: Synthetic oligonucleotide
tcctcatgggagcgcctatt

SEQ ID NO: 403 - DNA, 20 bp, Artificial Sequence
  Description of Artificial Sequence: Synthetic oligonucleotide
cctcatgggagcgcctattg

SEQ ID NO: 404 - DNA, 20 bp, Artificial Sequence
  Description of Artificial Sequence: Synthetic oligonucleotide
gcaggcaacagcagagccct

SEQ ID NO: 405 - DNA, 20 bp, Artificial Sequence
  Description of Artificial Sequence: Synthetic oligonucleotide
acccacatctacctgcagcc

SEQ ID NO: 406 - DNA, 20 bp, Artificial Sequence
  Description of Artificial Sequence: Synthetic oligonucleotide
cccacatctacctgcagccc

SEQ ID NO: 407 - DNA, 20 bp, Artificial Sequence
  Description of Artificial Sequence: Synthetic oligonucleotide
ccacatctacctgcagcccg

SEQ ID NO: 408 - DNA, 20 bp, Artificial Sequence
  Description of Artificial Sequence: Synthetic oligonucleotide
tcccctgccacagagaacga

SEQ ID NO: 409 - DNA, 20 bp, Artificial Sequence
  Description of Artificial Sequence: Synthetic oligonucleotide
cacagagaacgacggcgtga

SEQ ID NO: 410 - DNA, 20 bp, Artificial Sequence
  Description of Artificial Sequence: Synthetic oligonucleotide
gaacgacggcgtgatggcct

SEQ ID NO: 411 - DNA, 20 bp, Artificial Sequence
  Description of Artificial Sequence: Synthetic oligonucleotide
gaaggtcctggctgagttgc

SEQ ID NO: 412 - DNA, 20 bp, Artificial Sequence
  Description of Artificial Sequence: Synthetic oligonucleotide
ctgagttgctggagcagaag

SEQ ID NO: 413 - DNA, 20 bp, Artificial Sequence
  Description of Artificial Sequence: Synthetic oligonucleotide
gctggagcagaagaggaaaa

SEQ ID NO: 414 - DNA, 20 bp, Artificial Sequence
  Description of Artificial Sequence: Synthetic oligonucleotide
gcagaagaggaaaaaggctg

SEQ ID NO: 415 - DNA, 20 bp, Artificial Sequence
  Description of Artificial Sequence: Synthetic oligonucleotide
ctgtttcctcctccggaagc

SEQ ID NO: 416 - DNA, 20 bp, Artificial Sequence
  Description of Artificial Sequence: Synthetic oligonucleotide
tgtttcctcctccggaagcc

SEQ ID NO: 417 - DNA, 20 bp, Artificial Sequence
  Description of Artificial Sequence: Synthetic oligonucleotide
ccggaagccgggacaccgta

SEQ ID NO: 418 - DNA, 20 bp, Artificial Sequence
  Description of Artificial Sequence: Synthetic oligonucleotide
cggaagccgggacaccgtac

SEQ ID NO: 419 - DNA, 20 bp, Artificial Sequence
  Description of Artificial Sequence: Synthetic oligonucleotide
ggaagccgggacaccgtacg

SEQ ID NO: 420 - DNA, 20 bp, Artificial Sequence
  Description of Artificial Sequence: Synthetic oligonucleotide
ccgagaactcgtttactcaa

SEQ ID NO: 421 - DNA, 20 bp, Artificial Sequence
  Description of Artificial Sequence: Synthetic oligonucleotide
tagataacgtctttccaaaa

SEQ ID NO: 422 - DNA, 20 bp, Artificial Sequence
  Description of Artificial Sequence: Synthetic oligonucleotide
gagacaggggagtcccgact

SEQ ID NO: 423 - DNA, 20 bp, Artificial Sequence
  Description of Artificial Sequence: Synthetic oligonucleotide
caggggagtcccgacttgga

SEQ ID NO: 424 - DNA, 20 bp, Artificial Sequence
  Description of Artificial Sequence: Synthetic oligonucleotide
cttggaaggtgaatagttga

SEQ ID NO: 425 - DNA, 20 bp, Artificial Sequence
  Description of Artificial Sequence: Synthetic oligonucleotide
ggtgaatagttgaaggcaca

SEQ ID NO: 426 - DNA, 20 bp, Artificial Sequence
  Description of Artificial Sequence: Synthetic oligonucleotide
gtgaatagttgaaggcacat

SEQ ID NO: 427 - protein, 6 aa, Artificial Sequence
  Description of Artificial Sequence: Synthetic 6xHis tag
HHHHHH

(19) United States
(12) Patent Application Publication
(10) Pub. No.: US 2024/0101967 A1
(43) Pub. Date: Mar. 28, 2024

(54) COMPOSITIONS AND METHODS FOR GENE EDITING WITH WOOLLY MAMMOTH ALLELES

(71) Applicant: PRESIDENT AND FELLOWS OF HARVARD COLLEGE, Cambridge, MA (US)

(72) Inventors: George M. Church, Cambridge, MA (US); Eriona Hysolli, Cambridge, MA (US); Jessica Weber, Cambridge, MA (US); Pranam Chatterjee, Cambridge, MA (US); Cory Smith, Cambridge, MA (US)

(73) Assignee: PRESIDENT AND FELLOWS OF HARVARD COLLEGE, Cambridge, MA (US)

(21) Appl. No.: 18/266,093

(22) PCT Filed: Dec. 10, 2021
(86) PCT No.: PCT/US2021/062872

§ 371 Date: Jun. 8, 2023

Related U.S. Application Data
(60) Provisional application No. 63/123,616, filed on Dec. 10, 2020.

(51) Int. Cl. C12N 5/074; C12N 5/0775; C12N 9/22; C12N 15/11

(52) U.S. Cl. CPC C12N 5/0696; C12N 5/0662; C12N 9/22; C12N 15/11; C12N 2310/20; C12N 2510/00

(57) ABSTRACT

Described herein are compositions and methods for generating a viable cell that expresses at least one or more woolly mammoth genes. Also described herein are compositions and methods for generating an embryo, blastula, oocyte, or non-human organism that expresses one or more woolly mammoth genes.

### CROSS-REFERENCE TO RELATED APPLICATIONS

This application is a 35 U.S.C. § 371 National Phase Entry application of International Application No. PCT/US2021/062872 filed Dec. 10, 2021, which designates the U.S. and claims benefit under 35 U.S.C. § 119(e) of U.S. Provisional Application No. 63/123,616 filed Dec. 10, 2020, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jan. 27, 2022, is named 002806-098250WOPT_SL.txt and is 106,090 bytes in size.

### TECHNICAL FIELD

The technology described herein relates to gene edited, and/or reprogrammed mammalian cells, and uses thereof.

### BACKGROUND

There is currently an unmet need for the development of elephant tissue cultures, genome editing of non-human cells, and biological tools to aid animal conservation efforts. Synthetic biology and gene editing can improve treatments for wildlife diseases and rectify ecological imbalances caused by climate change, pollution, human consumption, hunting, human-caused disturbances, depletion of resources, and deforestation. Biobanking of tissues and cell lines from endangered and extinct species can cryopreserve them for future research well into the future. However, there is currently a lack of these tissues and cells from these species.

### SUMMARY

The compositions and methods described herein are based, in part, on the discovery that elephant somatic cells (e.g., Loxodonta africana cells) can be reprogrammed to a stem-cell-like phenotype, and can also be gene-edited to include one or more gene variant alleles from the extinct woolly mammoth (e.g., Mammuthus primigenius). The compositions and methods described herein provide a synthetic alternative to wildlife products and tools for understanding genetic diversity and cellular biology in endangered and extinct species.

In one aspect, described herein is a viable cell comprising at least one exogenous nucleic acid sequence selected from the group consisting of: the woolly mammoth genes listed in TABLE 1.

In one embodiment of any of the aspects, the cell expresses a polypeptide encoded by at least one nucleic acid sequence.

In another embodiment of any of the aspects, the cell is a reprogrammable cell.

In another embodiment of any of the aspects, the cell is a reprogrammed cell.

In another embodiment of any of the aspects, the cell is a stem cell. In another embodiment, the cell expresses at least one endogenous gene of a stem cell phenotype.

In another embodiment of any of the aspects, the stem cell is an induced pluripotent stem cell, embryonic stem cell, or mesenchymal stem cell.

In another embodiment of any of the aspects, the cell is a fibroblast cell or a mesenchymal cell.

In another embodiment of any of the aspects, the cell is selected from the group consisting of: a nerve cell, cartilage cell, bone cell, muscle cell, bone cell, fat cell, or epidermal cell.

In another embodiment of any of the aspects, the cell was previously differentiated in vitro into a cell selected from the group consisting of: a nerve cell, cartilage cell, bone cell, muscle cell, bone cell, fat cell, and an epidermal cell.

In another embodiment of any of the aspects, the cell does not express an endogenous homologue of the at least one woolly mammoth gene.

In another embodiment of any of the aspects, the cell is edited to inhibit expression of an endogenous homologue of the at least one woolly mammoth one gene.

In another embodiment of any of the aspects, the cell is a non-human cell.

In another embodiment of any of the aspects, the cell is an elephant cell.

In another embodiment of any of the aspects, the elephant cell is a Loxodonta africana (African elephant) cell or Elephas maximus (Asian elephant) cell.

In another embodiment of any of the aspects, the cell is a hyrax cell or manatee cell. In another embodiment of any of the aspects, the hyrax cell is selected from the group consisting of: a Dendrohyrax arboreus cell, a Dendrohyrax dorsalis cell, a Heterohyrax brucei cell, and a Procavia capensis cell. In another embodiment, the manatee cell is selected from the group consisting of: a Trichechus inunguis cell, a Trichechus manatus cell, a Trichechus manatus latirostris cell, a Trichechus manatus manatus cell, and a Trichechus senegalensis cell.

In another embodiment of any of the aspects, the cell is cryopreserved.

In another embodiment of any of the aspects, the cell was previously cryopreserved.

In another embodiment of any of the aspects, the cells exhibit a phenotype selected from the group consisting of: increased expression of one or more woolly mammoth polypeptides, modulation of calcium signaling, modulation of electrophysiological function, modulation of lipid composition of the cellular membrane, modulation of the rate of protein synthesis, and modulation of the rate of cell proliferation compared to an appropriate control, and, for stem cells, differentiation potential into other cell lineages.

In another aspect, described herein is an oocyte in which the endogenous nucleus has been replaced by the nucleus of a cell as described herein.

In another aspect, described herein is a non-wooly mammoth cell comprising at least one exogenous nucleic acid sequence selected from the group consisting of: the woolly mammoth genes listed in TABLE 1.

In another aspect, described herein is a gene-edited elephant cell comprising at least one exogenous nucleic acid sequence selected from the group consisting of: the woolly mammoth genes listed in TABLE 1, wherein the elephant cell is edited to alter or inactivate an elephant homologue of the at least one woolly mammoth gene.

In another aspect, described herein is an elephant cell comprising at least one guide RNA listed in TABLES 2 or 3. In one embodiment, the elephant cell further expresses an RNA-guided endonuclease guided by the at least one guide RNA.

In another aspect, described herein is a non-human cell comprising at least one guide RNA listed in TABLES 2 or 3. In one embodiment, the non-human cell further expresses an RNA-guided endonuclease guided by the at least one guide RNA.

In another aspect, described herein is a gene-edited elephant cell having the endogenous homologue of at least one gene selected from the group consisting of: the woolly mammoth genes listed in TABLE 1 that is edited to mimic the wooly mammoth variant of the homologue.

In one embodiment of any of the aspects, the cell is altered to delete or inhibit the function of the elephant homologue.

In another embodiment of any of the aspects, the stem cell marker is selected from the group consisting of: TRA 1-60, TRA 1-81, SSEA4, POU5F1, NANOG, REX1, hTERT, GDF3, miR-290 and mir-302 clusters among others.

In another embodiment, the cell comprises exogenous nucleic acid encoding one or more exogenous polypeptide(s) selected from the group consisting of: the woolly mammoth polypeptides listed in TABLE 1.

In another embodiment, the elephant homologue gene(s) corresponding to the one or more exogenous polypeptide(s) is/are inactivated.

In another aspect, described herein is a non-human organism comprising a viable cell as described herein.

In another aspect, described herein is a non-human embryo comprising a cell as described herein.

In another aspect, described herein is a non-human embryo comprising at least one exogenous nucleic acid sequence selected from the group consisting of: the woolly mammoth genes listed in TABLE 1.

In another aspect, described herein is a non-human oocyte comprising at least one exogenous nucleic acid sequence selected from the group consisting of: the woolly mammoth genes listed in TABLE 1.

In another aspect, described herein is a non-human 4-cell stage embryo comprising at least one exogenous nucleic acid sequence selected from the group consisting of: the woolly mammoth genes listed in TABLE 1.

In another aspect, described herein is a non-human 8-cell stage embryo comprising at least one exogenous nucleic acid sequence selected from the group consisting of: the woolly mammoth genes listed in TABLE 1.

In another aspect, described herein is a non-human blastula comprising at least one exogenous nucleic acid sequence selected from the group consisting of: the woolly mammoth genes listed in TABLE 1.

In another aspect, described herein is an enucleated non-human oocyte comprising a donor nucleus comprising the nucleic acid sequence of at least one gene selected from the group consisting of: the woolly mammoth genes listed in TABLE 1.

In another aspect, described herein is a non-human organism comprising the nucleic acid sequence of at least one gene selected from the group consisting of: the woolly mammoth genes listed in TABLE 1.

In one embodiment of any of the aspects, the embryo is a pre-gastrulation embryo.

In another embodiment of any of the aspects, the embryo is a chimeric embryo.

In another embodiment of any of the aspects, the embryo, blastula, or oocyte is cryopreserved.

In another embodiment of any of the aspects, the embryo, blastula, or oocyte was previously cryopreserved.

In another embodiment of any of the aspects, the non-woolly mammoth homologue of the exogenous nucleic acid sequence has been deleted or inactivated.

In another aspect, described herein is a guide RNA comprising a sequence selected from SEQ ID NO: 1 to SEQ ID NO: 426.

In another aspect, described herein is a nucleic acid encoding any of the guide RNAs described herein.

In one embodiment of any of the aspects, the nucleic acid encoding the guide RNA is operably linked to a nucleic acid sequence directing the expression of the guide RNA.

In another aspect, described herein is a vector comprising any of the nucleic acids described herein.

In another aspect, described herein is a cell comprising any of the guide RNAs described herein.

In another aspect, described herein is a cell comprising any of the nucleic acids described herein.

In another aspect, described herein is a cell comprising any of the vectors described herein.

In one embodiment of any of the aspects, the cell further comprises an RNA-guided endonuclease, the activity of which is guided by the guide RNA.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in biology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Genetics: Analysis of Genes and Genomes 9^{th} ed., published by Jones & Bartlett Publishers, 2014 (ISBN: 978-1284122930); Biology published by Pearson, 11^{th} ed. 2016, (ISBN: 0134093410); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, A D A M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein the term “stem cell” refers to a cell that can self-renew and differentiate to at least one more-differentiated or less developmentally-capable phenotype. The term “stem cell” encompasses stem cell lines, induced stem cells, non-human embryonic stem cells, pluripotent stem cells, multipotent stem cells, amniotic stem cells, placental stem cells, or adult stem cells. An “induced stem cell” is one derived from a non-pluripotent cell induced to a less-differentiated or more developmentally-capable phenotype by introduction of one or more reprogramming factors or genes. As the term is used herein, an induced stem cell need not be pluripotent, but has the capacity to differentiate, under appropriate conditions, to more than one more-highly-differentiated phenotype—it should be understood that that capacity was not present prior to the introduction of reprogramming factors. An induced stem cell will express at least one stem cell marker not expressed by the parent cell prior to the introduction of reprogramming factors. In this context, a stem cell marker is exclusive of a factor introduced for reprogramming. An induced pluripotent stem cell, or iPS cell, has the induced capacity to differentiate, under appropriate conditions, to a cell phenotype derived from each of the endoderm, mesoderm and ectoderm germ layers.

The term “marker” as used herein is used to describe a characteristic and/or phenotype of a cell. Markers can be used for selection of cells comprising characteristics of interest and can vary with specific cells. Markers are characteristics, whether morphological, structural, functional or biochemical (enzymatic) characteristics of the cell of a particular cell type, or molecules expressed by the cell type. In one aspect, such markers are proteins. Such proteins can possess an epitope for antibodies or other binding molecules available in the art. However, a marker can consist of any molecule found in or on a cell, including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method available to one of skill in the art. Markers can also be the absence of a morphological characteristic or absence of proteins, lipids etc. Markers can be a combination of a panel of unique characteristics of the presence and/or absence of polypeptides and other morphological or structural characteristics. In one embodiment, the marker is a cell surface marker.

As used herein, the phrase “expresses at least one stem cell marker” indicates that a cell expresses a marker, as the term is defined herein, that is characteristic of a stem cell as defined herein. The marker can be a particular morphology, but is more often expression of one or more polypeptides, whether on the cell surface or intracellular. The gain of expression of a stem cell marker will most often be accompanied by loss of expression of one or more markers of a differentiated phenotype. It should be understood that the “at least one stem cell marker” of a cell that “expresses at least one stem cell marker” is not a marker expressed from a construct exogenously introduced to the cell, but is expressed as part of the cell's response to the introduction of a reprogramming factor. Examples of stem cell markers include, but are not limited to TRA 1-60, TRA 1-81, SSEA4, POU5F1, NANOG, REX1, hTERT, GDF3, miR-290 and mir-302 clusters among others for embryonic stem cells, and differentiation markers like SOX2, MYOD, PAX6, NESTIN, NEUROGENIN1/2, CD34, IL-7, IL-3, NEUROD among many and depending on which differentiation lineage is preferred.

The term “exogenous” refers to a substance present in a cell that was introduced by the hand of man. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found. Alternatively, “exogenous” can refer to a nucleic acid or a polypeptide that has been introduced by a process involving the hand of man into a biological system such as a cell or organism in which it is found in relatively lower amounts and in which one wishes to increase the amount of the nucleic acid or polypeptide in the cell or organism, e.g., to create ectopic expression or levels.

The term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows: (Identical Deoxyribonucleotides.times.100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

As used herein, the term “reprogramming genes” or “reprogramming factors” refers to agents or nucleic acid molecules that can induce the reprogramming process in a somatic cell to re-express a less-differentiated, more stem-cell like phenotype. The reprogramming factor can be a nucleic acid, a polypeptide, or a small molecule that promotes a reprogrammed phenotype when introduced to a cell. Non-limiting examples of reprogramming factors include: Oct4 (Octamer binding transcription factor-4), SOX2 (Sex determining region Y)-box 2, Klf4 (Kruppel Like Factor-4), and c-Myc. These are the so-called “classical” or “standard” set of reprogramming factors used to derive, for example, induced pluripotent stem cells. Additional factors that can be considered reprogramming factors when introduced in the process of reprogramming cells to a less differentiated or stem cell phenotype include LIN28+Nanog, Esrrb, Pax5 shRNA, C/EBPα, p53 siRNA, UTF1, DNMT shRNA, Wnt3a, SV40 LT(T), hTERT), small molecule chemical agents including, but not limited to BIX-01294, BayK8644, RG108, AZA, dexamethasone, VPA, TSA, SAHA, PD0325901+CHIR99021(2i) and A-83-01. In some embodiments, the reprogramming genes or factors are Oct4, Klf4, SOX2, and c-Myc.

As used herein, the terms “dedifferentiation” or “retrodifferentiation” or “reprogramming” refer to a process that generates a cell that re-expresses a less differentiated phenotype than the cell from which it is derived and/or expresses at least one stem cell marker not expressed prior to that process. For example, a terminally-differentiated cell can be dedifferentiated to a multipotent cell. That is, dedifferentiation shifts a cell backward along the differentiation spectrum of totipotent cells to fully differentiated cells. Typically, reversal of the differentiation phenotype of a cell requires artificial manipulation of the cell, for example, by introducing or expressing exogenous polypeptide factors. Reprogramming is not typically observed under native conditions in vivo or in vitro.

As used herein, a “reprogrammed cell” is a cell that has been contacted with one or more reprogramming factors and expresses a less differentiated phenotype than the cell from which it was derived. The reprogrammed cell can also have the capacity to self-renew and will express at least one stem cell marker that was not delivered to the cell as a reprogramming factor. Furthermore, the reprogrammed cell will have the capacity to differentiate into a more-differentiated somatic cell type following differentiation protocols provided herein or described in the art.

As used herein, the term “somatic cell” refers to any cell other than a germ cell, a cell present in or obtained from a pre-implantation embryo, or a cell resulting from proliferation of such a cell in vitro. Stated another way, a somatic cell refers to any cells forming the body of an organism, excluding germ cells. Every cell type in the mammalian body—apart from the sperm and ova and the cells from which they are made (gametocytes)—is a somatic cell: internal organs, skin, bones, blood, and connective tissue are all substantially made up of somatic cells. In some embodiments the somatic cell is a “non-embryonic somatic cell,” by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro.

In the context of cell ontogeny, the term “differentiate”, or “differentiating” is a relative term that indicates a “differentiated cell” is a cell that has progressed further down the developmental pathway than its precursor cell. Thus in some embodiments, a stem cell as the term is defined herein, can differentiate to lineage-restricted precursor cells (such as a human cardiac progenitor cell or mid-primitive streak cardiogenic mesoderm progenitor cell), which in turn can differentiate into other types of precursor cells further down the pathway (such as a tissue specific precursor, such as a cardiomyocyte precursor), and then to an end-stage differentiated cell, which plays a characteristic role in a certain tissue type, and may or may not retain the capacity to proliferate further. Methods for in vitro differentiation of stem cells to other cell types are known in the art. Methods of differentiating stem cell-derived skeletal muscle cells, smooth muscle, and/or adipose cells are described, e.g., in U.S. Pat. No. 10,240,123 B2; and Cheng et al. Am J Physiol Cell Physiol (2014). Methods of differentiating kidney cells are described, e.g., in Tajiri et al. Scientific Reports 8:14919 (2018); Taguchi et al. Cell Stem Cell 14:53-67 (2014); and US application 2010/0021438 A1. Methods of differentiating cardiovascular cells are described, e.g., US Applicant No. 2017/0058263 A1; 2008/0089874 A1; 2006/0040389 A1; U.S. Pat. Nos. 10,155,927 B2; 9,994,812 B2; and 9,663,764 B2, Methods of differentiating endothelial cells (e.g., vascular endothelium) are described in, e.g., U.S. Pat. No. 10,344,262 B2, and Olgasi et al., Stem Cell Reports 11:1391-1406 (2018). Methods of differentiating hormone-producing cells are described, e.g., in U.S. Pat. No. 7,879,603 B2, and Abu-Bonsrah et al. Stem Cell Reports 10:134-150 (2018). Methods of differentiating bone cells are described, e.g., in Csobonyeiova et al. J Adv Res 8: 321-327 (2017), U.S. Pat. Nos. 7,498,170 B2; 6,391,297 B1; and US application No. 2010/0015164 A1. Methods of differentiating microglial cells are described, e.g., in WO 2017/152081 A1. Methods of differentiating epithelial cells and skin cells are described, e.g., in Kim et al., Stem Cell Research and Therapy (2018); U.S. Pat. Nos. 7,794,742 B2; 6,902,881 B2. Methods of differentiating blood cells and white blood cells are described, e.g., in U.S. Pat. Nos. 6,010,696 A and 6,743,634 B2. Methods of differentiating stem cell-derived beta cells are described, e.g., in WO 2016/100930A1. Each of the above references are incorporated herein by reference in their entireties.

As used herein, the term “cryopreserved” refers to a viable cell frozen in aqueous solution, where the aqueous solution is formulated to protect the cell during the freezing process.

The terms “decrease”, “reduce”, “reduction”, or “inhibit” are all used herein to mean a decrease by a statistically significant amount. In some embodiments, “reduce,” “reduction”, “decrease” or “inhibit” means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIG. 1 demonstrates the mammoth related species used to identify mammoth-specific traits. Adapted from Palkopoulou, et al. 2018, PNAS 115 (11) E2566-E2574.

FIG. 2 shows temperature ranges over which TRP genes are active. Adapted from Lynch et al., 2015, Cell Reports 12, 217-228.

FIG. 3 shows a multicistronic vector with cloned mammoth alleles. FIG. 3 discloses “6His” as SEQ ID NO: 427.

FIG. 4 shows the reprogramming overview and list of factors used for generating elephant iPSCs from elephant fibroblast cells. Reprogramming factors included Oct4, SOX2, KLF4, and cMyc.

FIG. 5 shows the pMPH86 vector used for reprogramming.

FIG. 6 shows a reprogramming vector.

FIG. 7 shows the initial reprogramming of elephant fibroblast cells to an induced phenotype having stem cell characteristics.

FIG. 8 shows Lox africana reprogrammed cells expanded in feeder-free conditions with MATRIGEL™.

FIG. 9 shows Principal Component Analysis (PCA) analysis of elephant cell populations.

FIG. 10 demonstrates a heatmap of various cell markers. The heatmap shows a comparison of stem cell markers that are high in elephant reprogrammed cells and low in fibroblast-like cells.

FIG. 11 shows differential expression analysis of differentiation markers that are high in elephant reprogrammed cells and low in differentiated parental populations.

FIG. 12 shows differential expression analysis of differentiation markers that are low in elephant reprogrammed cells and high in differentiated parental populations.

### DETAILED DESCRIPTION

Woolly mammoths (Mammuthus primigenius) were cold-tolerant members of the elephant family that once ranged across the vast mammoth steppe of the Northern Hemisphere in the last ice age, and became extinct across the majority of their range approximately 10,000 years ago. The woolly mammoth is arguably the best-characterized prehistoric animal, both through prehistoric art and from frozen remains found in Siberia and Alaska. These well-preserved specimens provide the rare opportunity to functionally characterize adaptive evolution in an extinct animal. Inhabitation of extreme environments, such as the cold regions of the northern latitudes, necessitates a suite of adaptive evolutionary changes. Genetic and morphological analyses of woolly mammoth specimens have revealed multiple physiological adaptations to cold, including dense, long hair, increased adipose tissue, decreased ears and tails, and hemoglobin structural polymorphisms. Studies of other cold-tolerant mammals have identified a number of convergent adaptations across the same genes and pathways, as well as unique adaptations to a shared environmental stressor.

The compositions and methods described herein are based, in part, on the discovery that cells (e.g., Loxodonta africana cells) can be modified to comprise and express alleles or homologues from the woolly mammoth (e.g., Mammuthus primigenius). In particular, viable cells can be gene-edited, whether by transfection, transduction or modification of existing elephant homologues to mimic the mammoth variants or alleles of the elephant genes. In some embodiments, the endogenous homologues of the mammoth genes are deleted or inactivated. Similar modifications to introduce woolly mammoth genes can be made to viable cells of other, non-human relatives of the elephant. The mammoth variants or alleles can modify the phenotype of the gene edited cells. Also described herein are oocytes, embryos, including chimeric embryos, and non-human organisms comprising such gene-edited cells. The compositions and methods described herein provide a synthetic alternative to wildlife products and new tools for understanding genetic diversity and cellular biology in endangered and extinct species of wildlife.

### Woolly Mammoth Genes

In one aspect, described herein is a viable cell comprising at least one exogenous nucleic acid sequence encoding a woolly mammoth gene, or comprising a modification of an endogenous gene to express a woolly mammoth homologue or variant of the endogenous gene. Of particular interest are genes that are shared by every woolly mammoth genome sequenced, which are not shared by any elephant genome (Asian or African) sequenced. By choosing genes in this manner, effects of individual variation within the group of woolly mammoth genomes sequenced and variations in Asian and/or African elephant genomes are minimized to focus on those variant sequences that are fully mammoth. In view of this, as used herein, a “woolly mammoth gene,” “woolly mammoth gene variant” or “woolly mammoth homologue” is a gene encoding a polypeptide that has a sequence encoded by all woolly mammoth genomes sequenced, and which differs from the homologous polypeptide encoded in all African and Asian elephant genomes sequenced. In this context, “differs from” refers to a difference of at least one amino acid relative to the homologous polypeptides encoded by the African or Asian elephant. A non-coding or regulatory nucleic acid sequence can be considered a “woolly mammoth sequence” if a non-coding motif of at least 20 nucleotides is present in every woolly mammoth genome sequenced, and not present in any Asian or African elephant genome sequenced. An Asian or African elephant gene or sequence modified by human intervention to encode a woolly mammoth gene or gene variant sequence is a woolly mammoth gene or gene or gene variant as the term is used herein. Where a woolly mammoth gene or gene variant as referred to herein is only found encoded in a woolly mammoth genome, and where the woolly mammoth is extinct, a woolly mammoth gene or gene variant sequence is necessarily exogenous to a viable cell; that is, the woolly mammoth gene or gene variant sequence is “exogenous” whether the sequence is in the cell through introduction of a foreign sequence or through gene editing an endogenous sequence to encode the woolly mammoth gene or gene variant sequence.

In one embodiment, the mammoth variant gene or genes is/are selected from the group consisting of: the woolly mammoth (e.g., Mammuthus primigenius) genes listed in TABLE 1.

Non-limiting examples of woolly mammoth genes that can be used are listed in the table below (TABLE 1). The woolly mammoth genes described herein are involved in a range of biological processes including but not limited to regulation of cold sensitivity, regulation of heat sensitivity, regulation of intracellular pH, regulation of axonogenesis and development, tRNA, metabolic processes, cellular adhesion, tissue development and formation, microtubule-based movement of cells, negative regulation of biological processes, gene expression, cellular macromolecule metabolic processes, and the like.

TABLE 1
WOOLLY MAMMOTH GENES

| Mammuthus | | Adaptive Phenotype; |
|---|---|---|
| Gene Name | Polypeptide Name(s) | Cellular Function |
| KRT8 | Keratin 8, Type II; KRT8 | Hair development |
| TRPM8 | Transient receptor potential cation | Decreased cold sensitivity; noxious |
| | channel subfamily M (melastatin) | cold sensing |
| | member 8 (TRPM8); cold and | |
| | menthol receptor 1 (CMR1) | |
| TRPV3 | Transient receptor potential cation | Decreased cold sensitivity; sense |
| | channel, subfamily V, member 3 | innocuous warmth. A mammoth- |
| | | specific substitution in TRPV3 |
| | | (N647D) occurring at a well- |
| | | conserved site seems to affect |
| | | thermosensation by mammoth TRPV3. |
| | | Associate to evolution of cold |
| | | tolerance, long hair, and large |
| | | adipose stores in mammoths. |
| TRPA1 | Transient receptor potential cation | Decreased cold sensitivity; sense |
| | channel, subfamily A, member 1; | noxious cold or heat depending on |
| | transient receptor potential ankyrin | species |
| | 1; TRPA1 | |
| TRPV4 | Transient receptor potential cation | Decreased cold sensitivity; heat |
| | channel subfamily V member 4 | sensitive but not known to be |
| | | involved in temperature sensation |
| PER2 | period circadian regulator 2 | Circadian biology; Transcriptional |
| | | repressor which forms a component |
| | | of the circadian clock |
| BMAL1 | Brain and Muscle ARNT-Like 1; | Circadian biology |
| | Aryl hydrocarbon receptor nuclear | |
| | translocator-like protein 1 | |
| | (ARNTL); BMAL1 | |
| HRH3 | Histamine H₃ receptor | Circadian biology |
| LEPR | Leptin receptor | Circadian biology; metabolism; |
| | | brown fat |
| CD109 | Cluster of Differentiation 109; | Sebaceous glands |
| | CD109; CPAMD7, p180, r150, | |
| | CD109 molecule | |
| BARX2 | BARX homeobox 1 | Sebaceous glands & Hair |
| RBL1 | Retinoblastoma-like 1 | Sebaceous glands & Hair |
| MKI67 | Marker of proliferation KI67; MKI67 | Hair development |
| BNC1 | Basonuclin (BNC); BNC1 | Hair development |
| POF1B | POF1B; actin-binding protein | Hair development |
| FREM1 | Fras1-related extracellular matrix | Hair development |
| | protein 1: FREM1 | |
| BMP2 | Bone morphogenetic protein 2; | Hair development |
| | BMP2 | |
| PRDM1 | PR Domain-containing prtein 1; | Hair development |
| | PRDM1 | |
| NES | Nestin; NES | Hair development |
| DLL1 | Delta-like canonical notch ligand 1; | Hair development |
| | DLL1 | |
| PTCH1 | Patched 1; PTCH1 | Hair development |
| SEMA5A | Semaphorin 5A; SEMA5A | Hair development |
| BHLHE22 | Basic helix-loop-helix family, | Hair development |
| | Member E22; BHLHE22 | |
| GLMN | glomulin; GLMN | Hair development |
| ACKR4 | Atypical chemokine receptor 4; | Hair development |
| | ACKR4 | |
| AKT1 | AKT serine/threonine kinase 1; | Hair development |
| | AKT1 | |
| SELENOP | Selenoprotein P; SELENOP | Hair development |
| NCAM1 | Neural Cell Adhesion Molecule 1 | Hair development |
| APOB | Apolipoprotein B; APOB | Lipid metabolism |
| ABCG8 | ATP-binding cassette sub-family G | Lipid metabolism |
| | member 8; ABCG8 | |
| CRP | C-reactive protein; CRP | Lipid metabolism |
| FABP2 | Fatty acid-binding protein 2; FABP-2; | Lipid metabolism |
| | FABP2 | |
| UCP1 | Uncoupling Protein 1; UCP1; | Brown fat; mitochondrial |
| | SLC25A7; Mitochondrial Brown | anion carrier protein |
| | Fat Uncoupling Protein 1; Solute | |
| | Carrier Family 25 Member 7; Thermogenin | |
| DLK1 | Delta Like Non-Canonical Notch | Brown fat |
| | Ligand 1; DLK1; Protein delta | |
| | homolog 1; Delta-like 1 homolog; | |
| | Preadipocyte factor 1 (Pref-1); | |
| | Fetal antigen (FA1) | |
| GHR | Growth hormone receptor; GHR | Brown fat |
| GPD2 | Glycerol-3-phosphate | Brown fat |
| | dehydrogenase 2; GPD2 | |
| HRH1 | Histamine Receptor H1 | Brown fat |
| LGALS12 | Galectin 12 | Brown fat |
| LPIN1 | Lipin-1 | Brown fat |
| MED13 | Mediator Complex Subunit 13; | Brown fat |
| | Thyroid Hormone Receptor- | |
| | Associated Protein Complex 240; | |
| | TRAP240; Thyroid Hormone Receptor- | |
| | Associated Protein 1; DRIP250 | |
| MLXIPL | MLX Interacting Protein Like | Brown fat |
| PDS5B | PDS5 Cohesin Associated Factor B; | Brown fat |
| | Androgen-Induced Proliferation | |
| | Inhibitor; AS3 | |
| SIK3 | SIK family kinase; Salt-Inducible | Brown fat |
| | Kinase 3; SIK3; Serine/Threonine- | |
| | Protein Kinase; QSK; | |
| ITPRID2 | ITPR Interacting Domain | Brown fat |
| | Containing 2; ITPRID2 | |
| COL27A1 | Collagen Type XXVII Alpha 1 | Domed cranium |
| | Chain; Collagen, Type XXVII, | |
| | Alpha 1; COL27A1 | |
| FIG4 | FIG4 Phosphoinositide 5- | Domed cranium |
| | Phosphatase; FIG4 | |
| HDAC4 | Histone Deacetylase 4; HDAC4 | Domed cranium |
| HTT | Huntingtin | Domed cranium |
| PFAS | Phosphoribosylformylglycinamidine | Domed cranium |
| | Synthase; PFAS | |
| PKD1 | Polycystin 1; Transient Receptor | Domed cranium |
| | Potential Cation Channel, | |
| | Subfamily P, Member 1; PKD1 | |
| SLX4 | SLX4 Structure-Specific | Domed cranium |
| | Endonuclease Subunit | |
| TCOF1 | Treacle Ribosome Biogenesis | Domed cranium |
| | Factor 1; Treacle | |
| TRIP1 | translation initiation factor 3 | Domed cranium |
| | subunit I; TRIP1 | |
| PHC1 | polyhomeotic homolog 1; PHC1 | Small tail bud |
| PHC2 | Polyhomeotic Homolog 2; PHC2 | Small tail bud |
| FN1 | Fibronectin 1; FN1 | Small tail bud |
| DACT1 | Dapper homolog 1; Dishevelled | Small tail bud |
| | Binding Antagonist Of Beta Catenin 1 | |
| HBB | Beta globin; β-globin; Hemoglobin β | Oxygen delivery |
| HBA | alpha-globin; α-globin; hemoglobin | Oxygen delivery |
| | A; adult hemoglobin; hemoglobin A1; | |
| HBA2 | alpha-globin 2; α-globin 2; | Oxygen delivery; variant of |
| | hemoglobin, alpha 2; HBA2; alpha | hemoglobin subunit A |
| | globin chain of hemoglobin; | |

The woolly mammoth genes described herein are common to all available woolly mammoth genome, but not found in any elephant genomes available. A database of woolly mammoth genes is also available on the world wide web at https://<usegalaxy.org/u/webb/p/mammoth>. See also, Lynch et al. Elephantid genomes reveal the molecular bases of Woolly Mammoth adaptations to the arctic. Cell Reports 12, 217-228, (2015), which is incorporated herein by reference in its entirety.

The woolly mammoth genes described herein can be used in any combination to be expressed in a viable cell as described herein. In some embodiments of any of the aspects, the at least one woolly mammoth nucleic acid sequence comprised by a viable cell encodes KRT8. In some embodiments of any of the aspects, the cell encodes and expresses woolly mammoth KRT8, and further encodes and expresses at least one exogenous woolly mammoth nucleic acid sequence selected from TABLE 1.

In another embodiment of any of the aspects, the cell comprises exogenous nucleic acid encoding one or more exogenous polypeptide(s) selected from the group consisting of: the woolly mammoth polypeptides listed in TABLE 1.

### Cell Preparations

The woolly mammoth genes described herein can be expressed by any viable cell that can accept exogenous genetic material. The cell can be, for example, a prokaryotic cell or a eukaryotic cell. In some embodiments, the cell is a eukaryotic cell. The cell can be a reprogrammed cell, a non-human oocyte, a cell of a non-human embryo or a cell of a non-human blastula. In some embodiments of any of the aspects, the cell is a fibroblast cell. In some embodiments, the cell is selected from the group consisting of: a nerve cell, a cartilage cell, a bone cell, a muscle cell, a bone cell, a fat cell, and an epidermal cell. In some embodiments, the cell was previously differentiated into a cell selected from the group consisting of: a nerve cell, cartilage cell, bone cell, muscle cell, bone cell, fat cell, and an epidermal cell.

The scientific literature provides guidance for one of ordinary skill in the art to isolate and prepare cells as necessary for use in the compositions and methods described herein. Sources of cells are discussed further herein below.

Cell sources: The cells described herein can be from any viable non-human source or organism. Usually the organism is an animal or vertebrate such as a wild animal, zoo animal, endangered animal, rodent, domestic animal, or bird. Animals can include, as non-limiting examples, an elephant, hippopatomus, hyrax, manatee, bear, panda, feline species, e.g., tiger, lion, cheetah, bobcat, canine species, e.g., fox, wolf, avian species, e.g., ostrich, emu, penguin, pigeon, and fish, e.g., trout, catfish, and salmon. In some embodiments, the cell described herein is from a mammal. Non-limiting examples of organisms from which cells can be derived include: elephants (e.g., Loxodonta africana, Elephas maximus, L. cyclotis); hyrax (e.g., Dendrohyrax arboreus, Dendrohyrax dorsalis, Heterohyrax brucei, Procavia capensis); and manatees (Trichechus inunguis, Trichechus manatus, Trichechus manatus latirostris, Trichechus manatus manatus, Trichechus senegalensis).

Elephant cells: In certain embodiments, a cell useful in the methods and compositions described herein is an elephant cell. In some embodiments, the cell is an elephant fibroblast cell. In some embodiments, the cell is an elephant stem cell. In some embodiments, the cell described herein is an elephant somatic cell reprogrammed to a stem cell or stem cell-like phenotype having stem cell-like morphology and/or expressing at least one stem cell marker described herein.

Elephant cells are unique among mammalian cells in exhibiting a high level of resistance to DNA damage. Perhaps for this reason, elephants have a lower rate of cancer than other mammalian species, including humans. See e.g., Abegglen et al. Potential Mechanisms for Cancer Resistance in Elephants and Comparative Cellular Response to DNA Damage in Humans. JAMA. (2015) 314(17): 1850-1860, which is incorporated herein by reference in its entirety. Abegglen determined that one mechanism of elephant cell resistance to DNA damage is that elephant cells have multiple copies of TP53, the gene encoding tumor suppressor p53. Tumor suppressor protein p53, plays an important role in regulating the cell cycle, apoptosis, and genomic stability of mammalian cells. p53 is also involved in the activation of DNA repair proteins and can arrest cell growth. Reprogramming of somatic cells to exhibit stem cell characteristics or pluripotency (so-called induced pluripotent stem, or iPS cells) is well established for cells of a wide range of eukaryotic and mammalian organisms. However, efforts to reprogram elephant cells to pluripotency have, to date, been unsuccessful. Without wishing to be bound by theory, it is thought that high levels of p53 expression in elephant cells may inhibit the genetic or epigenetic modifications necessary for reprogramming to a pluripotent stem cell phenotype. Manipulation of p53 expression or active gene copy number is contemplated as an approach for rendering elephant cells more amenable to reprogramming to a stem cell phenotype. Such manipulation can comprise transient expression knockdown, e.g., by RNA interference (RNAi) or related methods, or stable genome modification, e.g., by inactivation of one or more copies of p53 in the elephant genome (there are 20 copies of the p53 gene in the elephant genome). Such inactivation can include, for example, gene editing by, e.g., CRISPR or other method, to delete or interrupt one or more active copies of the p53 gene. Thus, in some embodiments, the viable cell described herein is a gene-edited elephant cell, which can include a cell edited to delete or inactivate one or more copies of TP53.

While not absolutely necessary for the introduction of exogenous gene sequences or manipulation of endogenous gene sequences in elephant cells, it is also contemplated that reducing p53 expression or gene copy number, alone or in combination with manipulation of other DNA damage sensors or DNA repair enzymes, can facilitate further genetic or epigenetic manipulation of elephant cells.

Described herein is the reprogramming of elephant somatic cells to a stem cell phenotype that has a stem cell morphology, and that expresses at least one stem cell marker. In some embodiments, the reprogrammed elephant cells form embryoid bodies or aggregate into clusters.

### Asasasas

Cell types: The cell described herein can be from any tissue isolated from an organism by methods known in the art. For example, placental tissue can be isolated from a given organism (e.g., an elephant), after full term delivery of young, and subsequently processed for cellular isolation and/or culture by methods known in the art. Additional exemplary cell types that can be used for the compositions and methods described herein include but are not limited to fibroblasts, skin cells, blood cells (e.g., leukocytes, monocytes, dendritic cells), stem cells, hematopoietic cells, liver cells, vascular cells, muscle cells, pancreatic cells, neural cells, ocular or retinal cells, epithelial or endothelial cells, lung cells, cardiac cells, intestinal cells, diaphragmatic cells, renal (i.e., kidney) cells, bone marrow cells, or any one or more selected tissues or cells of an organism for which genetic modification or gene editing to express a woolly mammoth gene is contemplated.

The cell can also be obtained from a cryopreserved viable tissue or cell sample. Thus, the cell described herein can be previously cryopreserved or can be progeny of a previously cryopreserved cell. Cells and tissues are frequently cryopreserved to temporally extend their viability and usefulness in biomedical applications. The process of cryopreservation involves, in part, placing cells into aqueous solutions containing electrolytes and chemical compounds that protect the cells during the freezing process (cryoprotectants). Such cryoprotectants are often small molecular weight molecules, such as glycerol, propylene glycol, ethylene glycol or dimethyl sulfoxide (DMSO), which prevent or limit intracellular ice crystal formation upon freezing of the cells. Protocols for both cryopreservation and thawing or re-establishing previously frozen cells in culture are known in the art, e.g., U.S. Pat. No. 9,877,475 B2; Karlsson J. O., Toner M. Long-term storage of tissues by cryopreservation: critical issues. Biomaterials. 1996; 17:243-256; and D.E. Principles of cryopreservation. Methods Mol Biol. 2007; 368:39-57, which are incorporated herein by reference in their entireties.

Stem cells: In certain embodiments, the compositions and methods described herein use or generate stem cells. Stem cells are cells that retain the ability to renew themselves through mitotic cell division and can differentiate into more specialized cell types. Three broad types of mammalian stem cells include: embryonic stem (ES) cells that are found in blastocysts, induced pluripotent stem cells (iPSCs) that are reprogrammed from somatic cells, and adult stem cells that are found in adult tissues. Other sources of stem cells can include, for example, amnion-derived or placental-derived stem cells. Pluripotent stem cells can differentiate into cells derived from any of the three germ layers.

Cells useful in the compositions and methods described herein can be obtained from essentially any somatic tissue, but where elephants or other species are endangered, efforts are taken to avoid any procedure that has the potential for causing long term harm to the animal. Where cells of, for example, an elephant are desired, one source of cells for manipulation, including, but not limited to introduction of woolly mammoth genes and testing for phenotypic effects of such genes, is post-partum placenta, which is normally delivered after delivery of a newborn. Placental tissues provide a rich source of viable cells that can be obtained without risk of harm to the animal, and are available, for example following birth of animals bred in captivity. In some embodiments, then, the cells described herein are obtained from the post-partum placenta of a species of animal. Where placenta and, for example, umbilical cord tissues and umbilical cord blood tend to be rich in stem cells, these tissues represent a source of cells, including elephant cells, that already have stem cell characteristics. While the stem cells in these elephant tissues are not pluripotent, it is specifically contemplated that where these tissues naturally include stem cells, placental or umbilical cord or umbilical cord blood stem cells can be used to derive even less differentiated stem cells, including pluripotent stem cells via reprogramming (see below for more on reprogramming to stem cell or pluripotent stem cell phenotypes). In some embodiments, the compositions and methods provided herein do not encompass generation or use of differentiated human cells derived from cells taken from a viable human embryo.

Embryonic stem cells: Cells derived from embryonic sources can include embryonic stem cells or stem cell lines obtained from a stem cell bank or other recognized depository institution. Other means of producing stem cell lines include methods comprising the use of a blastomere cell from an early stage embryo prior to formation of the blastocyst (at around the 8-cell stage). Such techniques use, for example, single cells removed in the pre-implantation genetic diagnosis technique routinely practiced in assisted reproduction clinics. A single blastomere cell can be co-cultured with established ES-cell lines and then separated from them to form fully competent ES cell lines. Analogous methods can be performed on early stage animal embryos produced, e.g., in the process of animal husbandry, e.g., through in vitro fertilization.

Embryonic stem cells and methods for their retrieval are described, for example, in Trounson A. O. Reprod. Fertil. Dev. (2001) 13: 523, Roach M L Methods Mol. Biol. (2002) 185: 1, and Smith A. G. Annu Rev Cell Dev Biol (2001) 17:435. The term “embryonic stem cell” is used to refer to the pluripotent stem cells of the inner cell mass of the embryonic blastocyst (see e.g., U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970).

Undifferentiated embryonic stem (ES) cells are easily recognized by those skilled in the art, and typically appear in the two dimensions of a microscopic view as colonies of cells having morphology including high nuclear/cytoplasmic ratios and prominent nucleoli. Endogenous polypeptide markers of embryonic stem cells include, for example, any one or any combination of Oct3, Nanog, SOX2, SSEA1, SSEA4 and TRA-1-60. In some embodiments, the cells for use in the methods and compositions described herein are not derived from embryonic stem cells or any other cells of embryonic origin.

In some embodiments of any of the aspects described herein, the cell described herein expresses at least one stem cell marker.

In some embodiments of any of the aspects, the stem cell marker is selected from the group consisting of TRA-1-60, POU5F1, NANOG.

Induced-pluripotent stem cells (iPSCs): In certain embodiments described herein, reprogramming of a differentiated somatic cell causes the differentiated cell to assume an undifferentiated state with the capacity for self-renewal and differentiation to cells of all three germ layer lineages. These are induced pluripotent stem cells (iPSCs or iPS cells).

Although differentiation is generally irreversible under physiological contexts, several methods have been developed in recent years to reprogram somatic cells to induced pluripotent stem cells. Exemplary methods are known to those of skill in the art and are described briefly herein below.

Methods of reprogramming somatic cells into iPS cells are described, for example, in U.S. Pat. Nos. 8,129,187 B2; 8,058,065 B2; US Patent Application 2012/0021519 A1; Singh et al. Front. Cell Dev. Biol. (February, 2015); and Park et al., Nature 451: 141-146 (2008); which are incorporated herein by reference in their entireties. Specifically, iPSCs are generated from somatic cells by introducing a combination of reprogramming transcription factors. The reprogramming factors can be introduced as, for example, proteins, nucleic acids (mRNA molecules, DNA constructs or vectors encoding them) or any combination thereof. Small molecules can also augment or supplement introduced transcription factors. While additional factors have been determined to affect, for example, the efficiency of reprogramming, a standard set of four reprogramming factors sufficient in combination to reprogram somatic cells to an induced pluripotent state includes Oct4 (Octamer binding transcription factor-4), SOX2 (Sex determining region Y)-box 2, Klf4 (Kruppel Like Factor-4), and c-Myc. Additional protein or nucleic acid factors (or constructs encoding them) including, but not limited to LIN28+Nanog, Esrrb, Pax5 shRNA, C/EBPα, p53 siRNA, UTF1, DNMT shRNA, Wnt3a, SV40 LT(T), hTERT) or small molecule chemical agents including, but not limited to BIX-01294, BayK8644, RG108, AZA, dexamethasone, VPA, TSA, SAHA, PD0325901+CHIR99021(2i) and A-83-01 have been found to replace one or the other reprogramming factors from the basal or standard set of four reprogramming factors, or to enhance the efficiency of reprogramming.

Reprogramming is a process that alters or reverses the differentiation state of a differentiated cell (e.g., a somatic cell). Stated another way, reprogramming is a process of driving the differentiation of a cell backwards to a more undifferentiated or more primitive type of cell. It should be noted that placing many primary cells in culture can lead to some loss of fully differentiated characteristics. However, simply culturing such cells included in the term differentiated cells does not render these cells non-differentiated cells or pluripotent cells. The transition of a differentiated cell to pluripotency requires a reprogramming stimulus beyond the stimuli that lead to partial loss of differentiated character when differentiated cells are placed in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. Thus, cells to be reprogrammed can be terminally differentiated somatic cells, as well as adult or somatic stem cells.

In some embodiments, reprogramming encompasses complete reversion of the differentiation state of a differentiated cell (e.g., a somatic cell) to a pluripotent state or a multipotent state. Reprogramming can result in expression of particular genes by the cells, the expression of which further contributes to reprogramming.

The efficiency of reprogramming (i.e., the number of reprogrammed cells) derived from a population of starting cells can be enhanced by the addition of various small molecules as shown by Shi, Y., et al. (2008) Cell-Stem Cell 2:525-528, Huangfu, D., et al. (2008) Nature Biotechnology 26(7):795-797, and Marson, A., et al. (2008) Cell-Stem Cell 3:132-135. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

Isolated iPSC clones can be tested for the expression of one or more stem cell markers. Such expression in a cell derived from a somatic cell identifies the cells as induced pluripotent stem cells. Stem cell markers can include but are not limited to SSEA3, SSEA4, CD9, Nanog, Oct4, Fbx15, Ecatl, Esgl, Eras, Gdf3, Fgf4, Cripto, Daxl, Zpf296, S1c2a3, Rexl, Utfl, and Natl, among others. In one embodiment, a cell that expresses Nanog and SSEA4 is identified as pluripotent.

In some embodiments of any of the aspects described herein, the cell described herein expresses at least one stem cell marker polypeptide or pluripotent stem cell marker polypeptide that the cell or its parent cells did not express prior to reprogramming. As used in this context, the new stem cell marker is not one encoded by an introduced nucleic acid sequence or construct, but is induced to be expressed following introduction of one or more reprogramming factors.

Methods for detecting the expression of such markers can include, for example, RT-PCR and immunological methods that detect the presence of the encoded polypeptides, such as Western blots, immunocytochemistry or flow cytometric analyses. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of isolated clones. The cells are introduced to nude mice and histology and/or immunohistochemistry using antibodies specific for markers of the different germ line lineages is performed on a tumor arising from the cells. The growth of a tumor comprising cells from all three germ layers, endoderm, mesoderm and ectoderm further indicates or confirms that the cells are pluripotent stem cells.

In some embodiments, a cell, such as an elephant cell, is treated to induce reprogramming, and produces a cell having a stem cell-like morphology distinct from the starting somatic cell and expressing one or more stem cell markers not expressed prior to reprogramming. Such markers are selected, for example, from stem cell markers TRA-1-60, SSEA4, POU5F1, and NANOG most prominently.

Mesenchymal stem cells (MSCs): In certain embodiments, a stem cell as described herein is a mesenchymal stem cell (MSC). Mesenchymal stem cells have the capacity to proliferate and to differentiate to muscle, skeletal (i.e. bone), blood, and vascular cell types and connective tissue, specifically osteoblasts, chondroblasts, adipocytes, fibroblasts, cardiomyoctes and skeletal myoblasts.

Mesenchymal stem cells can be recovered from bone marrow or adipose tissue of an adult organism described herein or cord blood of a neonate. These are referred to as mesenchymal stem cells (MSCs) because they can be cultured ex-vivo for a limited number of passages and be differentiated at the single cell level into mesodermal cell types as described above.

Methods of isolating, purifying and expanding mesenchymal stem cells (MSCs) are known in the art and include, for example, in U.S. Pat. No. 5,486,359 and Jones E. A. et al., 2002, Isolation and characterization of bone marrow multipotential mesenchymal progenitor cells, Arthritis Rheum. 46(12): 3349-60. A method of isolating mesenchymal stem cells from peripheral blood is described by Kassis et al [Bone Marrow Transplant. 2006 May; 37(10):967-76]. A method of isolating mesenchymal stem cells from placental tissue is described by Zhang et al. [Chinese Medical Journal, 2004, 117 (6):882-887]. Methods of isolating and culturing adipose tissue, placental and cord blood mesenchymal stem cells are described by Kern et al [Stem Cells, 2006; 24:1294-1301].

Embryonic stem cells (ESCs) can also be used as a source for generating MSCs. There are many methods to differentiate ESCs into MSCs known in the art. See, e.g., U.S. Pat. No. 9,725,698 B2; U.S. Pat. No. 5,486,359.

In some embodiments of any of the aspects described herein, the cell described herein expresses at least one MSC cell marker.

Markers for identifying MSCs include but are not limited to: Cluster of differentiation proteins including e.g., CD13, CD29, CD44, CD71, CD73, CD90, CD105, CD146, CD166, STRO-1, vimentin, and SSEA-4. Additional markers for MSCs and methods of culturing MSCs, as exemplified in human cells, but nonetheless applicable to non-human stem cell biology are reviewed, e.g., in Ullah I, et al. “Human mesenchymal stem cells—current trends and future prospective.” Biosci Rep. 2015; 35(2):e00191, which is incorporated herein by reference in its entirety.

Stem cells, induced pluripotent stem cells, induced mesenchymal stem cells or cells with induced stem cell morphology and expressing one or more stem cell markers have the capacity, when cultured under appropriate conditions, for differentiation to one or more different phenotypes. Thus, whether the somatic cells are reprogrammed to pluripotency or reprogrammed to a cell with induced, but more limited differentiation capacity, cells differentiated from the reprogrammed cells can be used, for example, to evaluate the phenotypic differences induced by the introduction of one or more woolly mammoth genes. For this purpose, the woolly mammoth gene(s) can be introduced prior to reprogramming of the cells to the less differentiated form. Alternatively, a woolly mammoth gene or genes can be introduced after the cells are reprogrammed and, for example, before they are re-differentiated to a desired phenotype.

In the context of cell ontogeny, the term “differentiate”, or “differentiating” is a relative term meaning a “differentiated cell” is a cell that has progressed further down the developmental pathway than its precursor cell. Thus, in some embodiments, a reprogrammed cell can differentiate to lineage-restricted precursor cells (such as a mesodermal stem cell), which in turn can differentiate into other types of precursor cells further down the pathway (such as a tissue specific precursor), and then to an end-stage differentiated cell, which plays a characteristic role in a certain tissue type, and may or may not retain the capacity to proliferate further.

In-vitro differentiated cells: Certain methods and compositions as described herein use cells that are differentiated in vitro from stem cells. Generally, throughout the differentiation process, a pluripotent cell will follow a developmental pathway along a particular developmental lineage, e.g., the primary germ layers—ectoderm, mesoderm, or endoderm.

The embryonic germ layers are the source from which all tissues and organs derive. For example, the mesoderm is the source of smooth and striated muscle, including cardiac muscle, connective tissue, vessels, the cardiovascular system, blood cells, bone marrow, skeleton, reproductive organs and excretory organs.

The germ layers can be identified by the expression of specific biomarkers and gene expression. Assays to detect these biomarkers include, e.g., RT-PCR, immunohistochemistry, and Western blotting. Non-limiting examples of biomarkers expressed by early mesodermal cells include HAND1, ESM1, HAND2, HOPX, BMP10, FCN3, KDR, PDGFR-α, CD34, Tbx-6, Snail-1, Mesp-1, and GSC, among others. Biomarkers expressed by early ectoderm cells include but are not limited to TRPM8, POU4F1, OLFM3, WNT1, LMX1A and CDH9, among others. Biomarkers expressed by early endoderm cells include but are not limited to LEFTY1, EOMES, NODAL and FOXA2, among others. One of skill in the art can determine which lineage markers to monitor while performing a differentiation protocol based on the cell type and the germ layer from which that cell is derived in development.

Induction of a particular developmental lineage in vitro is accomplished by culturing stem cells in the presence of specific agents or combinations thereof that promote lineage commitment. Generally, the methods described herein comprise the step-wise addition of agents (e.g., small molecules, growth factors, cytokines, polypeptides, vectors, etc.) into the cell culture medium or contacting a cell with agents that promote differentiation. For example, mesoderm formation is induced by transcription factors and growth factor signaling which includes but is not limited to VegT, Wnt signalling (e.g., via β-catenin), bone morphogenic protein (BMP) pathways, fibroblast growth factor (FGF) pathways, and TGFβ signaling (e.g., activin A). See e.g., Clemens et al. Cell Mol Life Sci. (2016), which is incorporated herein by reference in its entirety. Methods and agents that promote endoderm formation are described, e.g., in Loh et al. Cell Stem Cell 14(2) 237-252. (2014). Methods and agents that promote ectoderm formation are described, e.g., in Rogers et al. Birth Defects Res C Embryo Today 87(3): 249-262, (2009), Ozir et al., Wiley Interdicip. Rev Dev biol. 2(4): 479-498. (2013), and Sareen et al. J Comp Neurol 522(12) 2707-2728 (2014), which are incorporated herein by reference in their entireties.

Generally, in vitro-differentiated cells will exhibit a down-regulation of pluripotency or stem cell markers (e.g., HNF4-α, AFP, GATA-4, and GATA-6) throughout the step-wise process and exhibit an increase in expression of lineage-specific biomarkers (e.g., mesodermal, ectodermal, or endodermal markers). See for example, Tsankov et al. Nature Biotech (2015), which describes the characterization of human pluripotent stem cell lines and differentiation along a particular lineage. The differentiation process can be monitored for efficiency by a number of methods known in the art. This includes detecting the presence of germ layer biomarkers using standard techniques, e.g., immunocytochemistry, RT-PCR, flow cytometry, functional assays, optical tracking, etc.

### Methods for Introducing a Woolly Mammoth Gene to a Cell

In certain embodiments of any of the aspects, the cell compositions described herein express a polypeptide encoded by the at least one woolly mammoth nucleic acid sequence or gene (including, but not limited to the exogenous woolly mammoth genes in TABLE 1).

The cells described herein can be transfected, contacted with, or administered an exogenous woolly mammoth gene described herein by methods known in the art.

In some embodiments, the at least one nucleic acid sequence encoding a woolly mammoth gene is delivered via a vector.

A vector is a nucleic acid construct designed for delivery to a host cell or for transfer of genetic material between different host cells. As used herein, a vector can be viral or non-viral. The term “vector” encompasses any genetic element that is capable of replication when associated with the proper control elements and that can transfer genetic material to cells. A vector can include, but is not limited to, a cloning vector, an expression vector, a plasmid, phage, transposon, cosmid, artificial chromosome, virus, virion, etc.

In some embodiments of any of the aspects, the vector is selected from the group consisting of: a plasmid, a cosmid and a viral vector.

An expression vector is a vector that directs expression of an RNA or polypeptide (e.g., a woolly mammoth polypeptide) from nucleic acid sequences contained therein linked to transcriptional regulatory sequences on the vector. The sequences expressed will often, but not necessarily, be heterologous to the cell; a woolly mammoth gene introduced to a viable cell is heterologous to the cell. An expression vector may comprise additional elements, for example, the expression vector may have two replication systems, thus allowing it to be maintained in two organisms, for example in animal cells for expression and in a prokaryotic host for cloning and amplification. “Expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene.

In some embodiments, a vector is capable of driving expression of one or more sequences in a mammalian cell; i.e., the vector is a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant expression vector is capable of directing expression of the exogenous woolly mammoth nucleic acid sequence preferentially in a particular cell type (e.g., tissue-specific regulatory elements are used to express the nucleic acid in, for example, a hematopoietic cell or a hair follicle stem cell). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. hnmunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). While it can be useful to place woolly mammoth genes under the control of constitutive promoters to evaluate or quantitate their effect on cellular or tissue function, in certain embodiments, it can be advantageous to place exogenous woolly mammoth genes under the control of regulatory elements in a host cell that correspond to those connected to the woolly mammoth gene in its native context. Thus, to evaluate or quantitate the effect of a woolly mammoth hemoglobin gene or a woolly mammoth hair-related gene, as non-limiting examples, one would use regulatory elements that drive the respective homologues of those genes in cells of the host organism, e.g., hematopoietic cells or hair follicle stem cells. In addition, or alternatively, it can also be advantageous to modify the host cell's regulatory sequences for a given gene or sequence homologous to the woolly mammoth gene to be more similar to the mammoth regulatory sequence.

In some embodiments, the at least one nucleic acid sequence described herein is delivered to the cell described herein via an integrating vector. Integrating vectors have their delivered genetic material (or a copy of it) permanently incorporated into a host cell chromosome. Non-integrating vectors remain episomal which means the nucleic acid contained therein is never integrated into a host cell chromosome. Examples of integrating vectors include retroviral vectors, lentiviral vectors, hybrid adenoviral vectors, and herpes simplex viral vectors.

In some embodiments, the at least one nucleic acid sequence described herein is delivered to the cell described herein via a non-integrative vector. Non-integrative vectors include non-integrative viral vectors. Non-integrative viral vectors eliminate one of the primary risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. Containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free host cells. Other non-integrative viral vectors include adenoviral vectors and the adeno-associated viral (AAV) vectors.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages). This permits a self-limiting transient expression of a chosen heterologous gene or genes in a target cell. This aspect can be helpful, e.g., for the transient introduction of reprogramming factors, among other uses. As noted above, in some embodiments, the woolly mammoth nucleic acid sequence described herein is expressed in the cells from a viral vector. A “viral vector” includes a nucleic acid vector construct that includes at least one element of viral origin and has the capacity to be packaged into a viral vector particle. The viral vector can contain a nucleic acid encoding a polypeptide described herein in place of non-essential viral genes. The vector and/or particle can be utilized for the purpose of transferring nucleic acids into cells either in vitro or in vivo.

In certain embodiments, the woolly mammoth nucleic acid molecules described herein are introduced to a cell via a non-viral method. The nucleic acids described herein can be delivered using any transfection reagent or other physical means that facilitates entry of nucleic acids into a cell.

Methods of non-viral delivery of nucleic acids include lipofection, nucleofection, microinjection, electroporation, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

An “agent that increases cellular uptake” is a molecule that facilitates transport of a molecule, e.g., nucleic acid, or peptide or polypeptide, or other molecule that does not otherwise efficiently transit the cell membrane across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), or a polyamine (e.g., spermine). Further examples of agents that increase cellular uptake are disclosed, for example, in Winkler (2013). Oligonucleotide conjugates for therapeutic applications. Ther. Deliv. 4(7); 791-809.

In some embodiments of any of the aspects, the cell described herein, e.g., an elephant cell, is modified to express one or more woolly mammoth genes described herein. The one or more nucleic acid sequences encoding the woolly mammoth gene(s) can be delivered to the cell by any method discussed above or known in the art. Cell markers for the successful transfection of the cells described herein with the one or more nucleic acid sequences described herein are discussed further below.

### Methods of Inhibiting or Editing the Expression of an Endogenous Gene

In some embodiments of any the aspects, the cell described herein does not express an endogenous homologue of the at least one woolly mammoth gene described herein. In another embodiment of any of the aspects, the cell is edited to inhibit expression of an endogenous homologue of the at least one woolly mammoth gene.

In another embodiment of any of the aspects, the non-woolly mammoth homologue of the exogenous nucleic acid sequence has been deleted or inactivated.

It is contemplated herein that when one or more woolly mammoth genes are delivered to the host cell(s) it can be advantageous to modify the endogenous non-woolly mammoth homologue of the one or more genes to render the endogenous gene or genes non-functional. It is further contemplated herein that if two or more woolly mammoth genes are delivered to the host cell, one or both of the endogenous host cell genes would be altered. Thus, in this context, the host cell can comprise at least one non-functional endogenous homologue to the corresponding woolly mammoth gene.

In the context of elephant cells, the elephant homologue(s) of the one or more woolly mammoth genes to be expressed would be altered, deleted or inhibited such that only the one or more woolly mammoth genesis/are expressed by the cell. This can be achieved, for example, by standard gene editing of target sequences. It is also contemplated that rather than simply inactivating the endogenous gene, wholesale replacement of the endogenous gene, e.g. via homologous recombination, or via selective editing of the non-mammoth homologue gene(s) to encode and express the mammoth variant gene sequence(s) could also be effected.

The target sequence can be determined by methods known in the art. For example, sequence alignment tools can be used to compare the woolly mammoth nucleic acid sequences to those in the host organism, e.g., using NCBI Basic Local Alignment Sequence Tool (BLAST), OrthoMaM, Ensembl and/or Megalign (DNASTAR) software. Those skilled in the art can determine appropriate parameters for aligning sequences, including any algorithms needed to achieve maximal alignment over the full length of the sequences being compared.

Methods of inhibiting gene function in a host cell are known in the art. Non-limiting examples of gene knockdown, inhibition, and alteration include CRISPR/Cas9 systems, Transcription Activator-Like Effectors Nucleases (TALENS), and inhibitory nucleic acids. Exemplary embodiments of types of inhibitory nucleic acids can include, e.g., siRNA, shRNA, miRNA, and/or amiRNA, which are known in the art. One of ordinary skill in the art can design and test an inhibitory agent that targets the endogenous homologue of a woolly mammoth gene described herein.

Methods of preparing and delivering gene editing systems are described, e.g., in WO2015/013583A2; U.S. Pat. No. 10,640,789 B2; US Pg. No. US2019/0367948 A1; US Pg. No. 2017/0266320 A1; US Pg No. 2018/0171361 A1; US Pg. No. 2016/0175462 A1; and US Pg. No. 2018/0195089 A1, the contents of each of which are incorporated herein by reference in their entirety.

In general, CRISPR (clustered regularly interspaced short palindromic repeats) refers collectively to a gene modification system that uses enzymes and factors derived from a prokaryotic defense mechanism against bacteriophages to precisely modify target gene sequences in a given cell type. CRISPR gene editing systems can include transcripts and other elements involved in the expression of or directing the activity of CRISPR-associated (“Cas”) genes, including sequences encoding a Cas nuclease gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes.

A guide sequence of the CRISPR system is designed to have complementarity to a target sequence (e.g., an elephant homologue of one more of the woolly mammoth genes described herein). A target sequence may comprise any DNA, RNA polynucleotide sequence. Hybridization between the target sequence and a guide sequence promotes the formation of a CRISPR complex. The guide sequence hybridized to a target sequence and complexed with one or more Cas proteins results in cleavage of one or both strands in or near (e.g. within 1, 2, 3, 4, 5, 6, 7, 8, 9, 10, 20, 50, or more base pairs from) the target sequence. Full complementarity between the target sequence and the guide sequence is not necessarily required, provided there is sufficient complementarity to cause hybridization and promote formation of a CRISPR complex.

When editing of a gene is desired, an editing sequence or an editing template polynucleotide may be used for recombination into the targeted locus comprising the target sequences. In some embodiments, the recombination is homologous recombination. For example, an elephant homologue of the woolly mammoth gene can be altered or deleted and replaced with one or more of the woolly mammoth gene sequences described herein.

Base editing is another approach to alter an endogenous gene described herein. Base editing can be used to introduce point mutations in cellular DNA or RNA without making double-stranded breaks. In some embodiments, the method of altering an endogenous nucleic acid described herein is by cytosine base editing, adenine base editing, antisense-oligonucleotide-directed A to I RNA editing, or Cas 13 base editing. Methods of base editing are known in the art and described, e.g., in Rees et al. Nature Rev Genet. 19(12); 770-788 (2018) and Kopmor et al. Nature 533, 420-424 (2016), which are incorporated herein by reference in their entireties.

CRISPR system or base editing elements can be combined in a single vector and may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a cell as described herein is transiently transfected with the components of a gene editing system (such as by transient transfection of one or more vectors, or transfection with RNA), and modified through the activity of a CRISPR or base editing complex, to establish a new cell or cell line comprising cells containing a modification to the host cell gene.

In some embodiments, the cell described herein is a gene-edited elephant cell. In some embodiments, one or more elephant genes have been altered to encode one or more of the woolly mammoth genes described herein.

Provided herein is an elephant cell comprising at least one guide RNA listed in TABLES 2 or 3. In one embodiment, the elephant cell comprises at least 2; at least 3; at least 4; at least 5; at least 6; at least 7; at least 8; at least 9; at least 10; at least 11; at least 12; at least 13; at least 14; at least 15; at least 16; at least 17; at least 18; at least 19; at least 20; at least 21; at least 22; at least 23; at least 24; at least 25; at least 26; at least 27; at least 28; at least 29; at least 30; at least 31; at least 32; at least 33; at least 34; at least 35; at least 36; at least 37; at least 38; at least 39; at least 40; at least 41; at least 42; at least 43; at least 44; at least 45; at least 46; at least 47; at least 48; at least 49; at least 50; at least 51; at least 52; at least 53; at least 54; at least 55; at least 56; at least 57; at least 58; at least 59; at least 60; at least 61; at least 62; at least 63; at least 64; at least 65; at least 66; at least 67; at least 68; at least 69; at least 70; at least 71; at least 72; at least 73; at least 74; at least 75; at least 76; at least 77; at least 78; at least 79; at least 80; at least 81; at least 82; at least 83; at least 84; at least 85; at least 86; at least 87; at least 88; at least 89; at least 90; at least 91; at least 92; at least 93; at least 94; at least 95; at least 96; at least 97; at least 98; at least 99; at least 100 or more guide RNAs listed in TABLES 2 and/or 3. Where the elephant cell expresses more than 1 guide RNA (i.e., at least 2 guide RNAs), the expression of the at least 2 guide RNAs can be done concurrently or sequentially.

In one embodiment, the elephant cell further expresses an RNA-guided endonuclease guided by the at least one guide RNA. RNA-guided endonucleases are well known in the art and exemplary endonucleases are described herein.

Also provided herein is a non-human cell comprising at least one guide RNA listed in TABLES 2 or 3. In one embodiment, the non-human cell comprises at least 2; at least 3; at least 4; at least 5; at least 6; at least 7; at least 8; at least 9; at least 10; at least 11; at least 12; at least 13; at least 14; at least 15; at least 16; at least 17; at least 18; at least 19; at least 20; at least 21; at least 22; at least 23; at least 24; at least 25; at least 26; at least 27; at least 28; at least 29; at least 30; at least 31; at least 32; at least 33; at least 34; at least 35; at least 36; at least 37; at least 38; at least 39; at least 40; at least 41; at least 42; at least 43; at least 44; at least 45; at least 46; at least 47; at least 48; at least 49; at least 50; at least 51; at least 52; at least 53; at least 54; at least 55; at least 56; at least 57; at least 58; at least 59; at least 60; at least 61; at least 62; at least 63; at least 64; at least 65; at least 66; at least 67; at least 68; at least 69; at least 70; at least 71; at least 72; at least 73; at least 74; at least 75; at least 76; at least 77; at least 78; at least 79; at least 80; at least 81; at least 82; at least 83; at least 84; at least 85; at least 86; at least 87; at least 88; at least 89; at least 90; at least 91; at least 92; at least 93; at least 94; at least 95; at least 96; at least 97; at least 98; at least 99; at least 100 or more guide RNAs listed in TABLES 2 and/or 3. Where the non-human cell expresses more than 1 guide RNA (i.e., at least 2 guide RNAs), the expression of the at least 2 guide RNAs can be done concurrently or sequentially.

TABLES 2 and 3 include exemplary point mutations identified herein between certain African elephant and Woolly mammoth genes, as well as gene-editing methods for altering the African elephant gene to mimic the Wooly mammoth gene. For example, TABLES 2 and 3 provide guide RNAs sequences for various gene editing tools (i.e., CRISPR Cas-9 and SpRYC) that will generate the identified point mutation. “SpRYC” refers to a variant engineered from SpCas9-VRQR designed to recognize virtually all PAM sequences, and is exceptionally effective at base editing. SpRY is further described in, e.g., Zhang, D. and Shang, B. SpRY: Engineered CRISPR/Cas9 Harnesses New Genome-Editing Power. Trends Genet. 2020 August; 36(8):546-548; which is incorporated herein by reference in its entirety.

Further provided herein is a guide RNA comprising a sequence selected from SEQ ID NO: 1 to SEQ ID NO: 426.

Also provided herein is a cell comprising any of the guide RNAs described herein. In one embodiment, the cell further comprises an RNA-guided endonuclease, the activity of which is guided by the guide RNA.

Also provided herein is a nucleic acid encoding any of the guide RNAs described herein. In one embodiment, the nucleic acid encoding the guide RNA is operably linked to a nucleic acid sequence directing the expression of the guide RNA.

Also provided herein is a vector comprising any of the nucleic acids described herein.

Also provided herein is a cell comprising any of the nucleic acids described herein. In one embodiment, the cell further comprises an RNA-guided endonuclease, the activity of which is guided by the guide RNA.

Also provided herein is a cell comprising any of the vectors described herein. In one embodiment, the cell further comprises an RNA-guided endonuclease, the activity of which is guided by the guide RNA.

### Woolly Mammoth Gene Expression and Phenotypes

The compositions and methods described herein can be used to express a woolly mammoth gene in a viable non-human cell. In some embodiments of any of the aspects, an elephant cell expresses one or more of the woolly mammoth genes in TABLE 1.

In some embodiments of any of the aspects, a cell as described herein exhibits a phenotype associated with the cellular function or expression of the woolly mammoth gene or genes described herein (e.g., those in TABLE 1).

Woolly mammoth phenotypes can be distinguished from the host cell phenotype by any method known in the art, e.g., via morphology (e.g., via microscopy), immunohistochemistry, electrophysiological recordings, metabolic assays, RT-PCR, proteomics, or sequencing analysis.

Expression of genes indicative of a given phenotype (e.g., one or more of the woolly mammoth genes in TABLE 1) can be determined by detection or measurement of RNA and/or protein using standard methods.

Metabolic assays can be used to determine the differentiation stage and/or the functional phenotypes of the cells described herein. For example, the woolly mammoth genes described herein can modulate processes such as the rate of protein synthesis and ATP production in a given cell. Non-limiting examples of metabolic assays include cellular bioenergetics assays (e.g., Seahorse Bioscience XF Extracellular Flux Analyzer™), and oxygen consumption tests. Specifically, cellular metabolism can be quantified by oxygen consumption rate (OCR), OCR trace during a fatty acid stress test, maximum change in OCR, maximum change in OCR after FCCP addition, and maximum respiratory capacity among other parameters. Furthermore, a metabolic challenge or lactate enrichment assay can provide a measure of cellular maturity, differentiation stage, or a measure of the effects of various nucleic acid sequences delivered to such cells. Brown fat thermogenesis is measured through, e.g., UCP1 and HIF1a activity, via, for example, expression, fluorescence, or bioluminescence assays.

The woolly mammoth genes described herein can alter the electrophysiological properties of a host cell. Non-limiting examples of genes that can alter the electrophysiological properties of the cell described herein include: TRPM8, TRPV3, TRPA1, and TRPV4.

Methods of measuring electrophysiological function of a cell are known in the art. Non-limiting examples of such methods to determine electrophysiological function of a cell include whole cell patch clamp (manual or automated), multielectrode arrays, field potential stimulation, calcium imaging and optical mapping, among others. Cells can be electrically stimulated during whole cell current clamp or field potential recordings to produce an electrical response. Measurement of field potentials and biopotentials of the cells described herein can be used to determine the differentiation stage and/or woolly mammoth phenotypes.

Methods of detecting transient receptor potential (TRP) channel activity are known in the art and are described e.g., in Samanta et al. Subcell Biochem. 2018; 87: 141-165 and Talavera and Nilius, TRP Channels. Ch. 11. Boca Raton (FL): CRC Press/Taylor & Francis; 2011, which are incorporated herein by reference in their entireties. The majority of TRP channels are permeable to calcium (Ca2+), and therefore constitute Ca2+ entry pathways in multiple cell types. Accordingly, in some embodiments, the phenotype of a cell described here involves a modulation of calcium signals and/or a modulation of electrophysiological function compared to an appropriate control.

In certain embodiments, the phenotype of a cell described herein involves a modulation of lipid composition of the cellular membrane, as compared to an appropriate control. In some embodiments, the phenotype of a cell described herein involves a modulation of the rate of protein synthesis, and/or modulation of the rate of cell proliferation, transcriptomic profile, and differentiation potential (for a stem cell) compared to an appropriate control.

The lipid composition of a cell membrane can be determined e.g., by liquid chromatography-mass spectrometry (LC-MS) or electrospray ionization (ESI). Methods of measuring protein synthesis rate are discussed, e.g., in Princiotta et al. Immunity Vol 18, 343-354, (2003), which is incorporated herein by reference in its entirety. Cell proliferation rate can be determined using commercially available kits or flow cytometry, e.g., kits sold by ThermoFisher Scientific® (Catalog number: C34564) or Roche® (Cell Proliferation Kit I (MTT), Catalog #11465007001).

One of skill in the art can determine the appropriate assay to detect and measure alterations in a particular cellular phenotype. The results of the assay can be compared to an appropriate control cell. In some embodiments, the appropriate control cell is a cell that has not been modified to include or express a woolly mammoth gene described herein.

### Genetically Modified Oocytes, Blastulas, and Non-Human Organisms

The reconstruction of embryos by the transfer of a nucleus from a donor cell (e.g., an embryo) to an enucleated oocyte or one cell zygote allows the production of genetically identical individuals. Somatic cell nuclear transfer or SCNT is a laboratory procedure known in the art for the reconstruction and reproduction of organisms, e.g., mammals. This has clear advantages for both research and also in commercial applications (i.e. multiplication of genetically valuable livestock, uniformity of wildlife products, animal management, and ecological preservation efforts).

The compositions described herein can be generated by modifying the chromatin of a donor cell prior to nuclear transfer and/or nuclear transfer procedures.

The donor cell in each instance is modified to encode and express a woolly mammoth gene as described herein. In some embodiments of any of the aspects, the donor cell is a somatic cell. In some embodiments of any of the aspects, the donor cell is an elephant somatic cell. In some embodiments of any of the aspects, the donor cell is a fetal fibroblast cell. In some embodiments of any of the aspects, the donor cell is an elephant fetal fibroblast cell. In some embodiments of any of the aspects, the donor cell is a stem cell, including, but not limited to an adult stem cell, an induced stem cell, a stem cell derived or obtained from placenta, umbilical cord or umbilical cord blood, or a cell induced, e.g., via reprogramming, to a stem cell morphology and expressing at least one stem cell marker. The donor cell can be modified to reduce, inhibit or inactivate the expression of an endogenous gene corresponding to the woolly mammoth gene introduced.

In some embodiments of any of the aspects, the recipient cell is a non-human oocyte. In some embodiments of any of the aspects, the recipient cell is a non-human mammalian oocyte. In some embodiments of any of the aspects, the recipient cell is an elephant oocyte, a hyrax oocyte, or a manatee oocyte.

In some embodiments of any of the aspects, the recipient cell has had its genetic material or nucleus removed. Thus, described herein is an oocyte in which the endogenous nucleus has been replaced by the nucleus of a cell described herein. In another aspect, described herein is a non-human oocyte comprising at least one exogenous nucleic acid sequence selected from the group consisting of: the woolly mammoth genes listed in TABLE 1.

Methods of nuclear transfer are known in the art and described, e.g., in U.S. Pat. No. 7,355,094 B2, U.S. Pat. No. 7,332,648 B2, WO 1996/007732 A1, Keefer et al., Biol. Reprod. 50 935-939 (1994), Sims & First, PNAS 90 6143-6147 (1994)), Smith & Wilmut, Biol. Reprod. 40 1027-1035 (1989), and Wilmut et al. Nature 385, 810-813 (1997), R. P. Lanza, et al. Cloning of an endangered species (Bos gaurus) using interspecies nuclear transfer. Cloning, 2 (2000), pp. 79-90, M. C. Gomez, et al. Birth of African wildcat cloned kittens born from domestic cats. Cloning Stem Cells, 6 (2004), pp. 247-258, B. C. Lee, Dogs cloned from adult somatic cells. Nature, 436 (2005), p641, D. Shi et al., Buffalos (Bubalus bubalis) cloned by nuclear transfer of somatic cells. Biol Reprod, 77 (2007), pp. 285-291, N. A. Wani, et al. Production of the first cloned camel by somatic cell nuclear transfer. Biol Reprod, 82 (2010), pp. 373-379, which are incorporated herein by reference in their entireties. Methods of modifying the donor cell prior to SCNT are reviewed, e.g., in Rodriguez-Osorio et al. “Reprogramming mammalian somatic cells.” Theriogenology 78:9 (2012) 1869-1886, Loi et al., Genetic rescue of an endangered mammal by cross-species nuclear transfer using post-mortem somatic cells. Nat Biotechnol, 19 (2001), 962-964, In general, nuclear transfer is performed under a microscope with a thin needle or micropipette capable of extracting a nucleus from a donor cell (e.g., a somatic cell) and a host cell with a vacuum. Alternatively, a drill is used to pierce the outer layers of a cell to remove the nucleus. Once the nucleus of the donor and host cell are removed, the donor nucleus can replace the nucleus of the host cell (e.g., an oocyte). In another method, the host cell nucleus is removed and the donor somatic cell is fused with the empty host cell by electrical pulsing.

The genetic material from the donor cell allows for the reprogramming of the recipient (host) cell. In this context, reprogramming is not a process of reversing differentiation, but rather, a process of altering the entire genetic program of an oocyte to that encoded by a donor nucleus. Various strategies have been employed to improve the success rate of SCNT. Most of these focus on the donor cell, including: 1) cell type, or tissue of origin; 2) passage number; 3) cell cycle stage; and 4) use of chemical agents and cellular extracts to modify the donor cell's epigenetic state. See e.g., Hill et al. Development rates of male bovine nuclear transfer embryos derived from adult and fetal cells. Biol Reprod, 62 (2000), pp. 1135-1140, Kato et al. Cloning of calves from various somatic cell types of male and female adult, newborn and fetal cows. J Reprod Fertil, 120 (2000), pp. 231-237, Jones et al. DNA hypomethylation of karyoplasts for bovine nuclear transplantation. Mol Reprod Dev, 60 (2001), pp. 208-213, B. P. Enright et al. Methylation and acetylation characteristics of cloned bovine embryos from donor cells treated with 5-aza-2′-deoxycytidine. Biol Reprod, 72 (2005), pp. 944-948, Liu et al. Hypertonic medium treatment for localization of nuclear material in bovine metaphase II oocytes. Biol Reprod, 66 (2002), pp. 1342-1349, Yamanaka et al. Gene silencing of DNA methyltransferases by RNA interference in bovine fibroblast cells. J Reprod Dev, 56 (2010), pp. 60-67, and Wang et al. Sucrose pretreatment for enucleation: an efficient and non-damage method for removing the spindle of the mouse MII oocyte. Mol Reprod Dev, 58 (2001), pp. 432-436, which are incorporated herein by reference in their entireties.

Non-limiting examples of such reagents and conditions include microtubule inhibitors (e.g., nocodazole), cytochalasin B, DNA methyl-transferase inhibitors, trichostatin A, 5-aza-2′-deoxycytidine, knock down of DNMT1 gene expression, and direct current (DC) pulsing.

The oocyte bearing a modified donor nucleus as described herein can be stimulated to divide and form early-stage embryos. This process can be achieved by culturing the cells in medium comprising growth factors (e.g., as described in Wu et al., Cell. 168, 473-486 (2017), which is incorporated herein by reference in its entirety). Described herein is a non-human embryo comprising a cell or a population of cells described herein. In another aspect, described herein is a non-human embryo comprising at least one exogenous nucleic acid sequence selected from the group consisting of: the woolly mammoth genes listed in TABLE 1. In some embodiments of any of the aspects, the embryo comprises or is comprised of elephant cells comprising at least one exogenous nucleic acid sequence selected from the group consisting of: the woolly mammoth genes listed in TABLE 1.

The non-human embryos described herein can be implanted into the uterus of a female non-human organism (e.g., a female elephant) by embryo transfer or the embryos can be cultured under conditions that permit the formation of blastulas. Embryo transfer can be performed by a skilled practitioner at any stage of embryogenesis, including blastocyst stage. Methods of selecting and transferring an embryo or blastula into an organism are known in the art. See e.g., Mains L, Van Voorhis B J (August 2010). “Optimizing the technique of embryo transfer”. Fertility and Sterility. 94 (3): 785-90, Meseguer M, Rubio I, Cruz M, Basile N, Marcos J, Requena A (December 2012). “Embryo incubation and selection in a time-lapse monitoring system improves pregnancy outcome compared with a standard incubator: a retrospective cohort study”. Fertility and Sterility. 98 (6): 1481-9.e10, and Mullin C M, Fino M E, Talebian S, Krey L C, Licciardi F, Grifo J A (April 2010). “Comparison of pregnancy outcomes in elective single blastocyst transfer versus double blastocyst transfer stratified by age”. Fertility and Sterility. 93 (6): 1837-43, which are incorporated herein by reference in their entireties.

In instances where there may be constraints on the development of a nuclear transplanted oocyte-derived embryo to term, it may be preferable to generate a chimeric non-human organism formed from cells derived from a naturally formed embryo and an embryo modified by oocyte nuclear transfer. Such a chimera can be formed by taking a population of cells of the natural embryo and a population of the cells of the embryo modified by oocyte nuclear transfer at any stage up to the blastocyst stage and forming the new embryo by aggregation or injection. The proportion of added cells may be in the ratio of about 50:50 or another suitable ratio to achieve the formation of an embryo which develops to term. The presence of wild-type cells (e.g., cells not expressing a woolly mammoth gene described herein) in these circumstances is contemplated herein to assist in rescuing the reconstructed embryo and allowing successful development to term and a live birth of the non-human organism. Furthermore, the reconstituted embryo can be cultured, in vivo or in vitro to blastocyst. Additional protocols for forming chimeras are discussed, e.g., in U.S. Pat. No. 7,232,938 B2.

A blastula is a hollow sphere of cells formed during an early stage of embryonic development in animals. Described herein is a non-human blastula comprising at least one exogenous nucleic acid sequence selected from the group consisting of: the woolly mammoth genes listed in TABLE 1. In some embodiments of any of the aspects, the blastula is comprised of elephant cells that express one or more woolly mammoth genes described herein.

Markers for the blastula stage during embryogenesis are known in the art and are discussed e.g., in Lombardi, Julian (1998). “Embryogenesis”. Comparative vertebrate reproduction. Springer. p. 226. Methods of culturing and generating blastulas are discussed, e.g., by Latham et al. Alterations in Protein Synthesis Following Transplantation of Mouse 8-Cell Stage Nuclei to Enucleated 1-Cell Embryos, Developmental Biology. Vol 163, Issue 2, (1994) and Ng. et al. Epigenetic memory of active gene transcription is inherited through somatic cell nuclear transfer. Proc Natl Acad Sci USA, 102 (2005), pp. 1957-1962, which are incorporated herein by reference in their entireties.

Upon the successful transfer of an embryo or blastula described herein by the methods discussed above, embryonic development of the organism described herein can be permitted to progress, e.g., to gastrulation or further development. Such development can permit the generation of a live, genetically modified non-human organism that comprises one or more cells comprising and expressing one or more woolly mammoth genes as described herein. Described herein is an elephant comprising one or more cells expressing at least one exogenous nucleic acid sequence selected from the group consisting of: the woolly mammoth genes listed in TABLE 1.

It is to be understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The technology provided herein can be further be described by any of the numbered paragraphs herein below.
1) A viable cell comprising at least one exogenous nucleic acid sequence selected from the group consisting of: the woolly mammoth genes in TABLE 1.
2) The cell of paragraph 1, wherein the cell expresses a polypeptide encoded by the at least one nucleic acid sequence.
3) The cell of any of the preceding paragraphs, wherein the cell is a stem cell.
4) The cell of any of the preceding paragraphs, wherein the cell expresses at least one stem cell marker.
5) The cell of any of the preceding paragraphs, wherein the stem cell marker is selected from NANOG, SSEA1, SSEA4, or TRA-1-60.
6) The cell of any of the preceding paragraphs, wherein the stem cell is an induced stem cell, embryonic stem (ES) cell, or mesenchymal stem cell (MSC).
7) The cell of any of the preceding paragraphs, wherein the cell is a reprogrammed cell.
8) The cell of any of the preceding paragraphs, wherein the cell is a fibroblast cell or a mesenchymal cell.
9) The cell of any of the preceding paragraphs, wherein the cell is selected from the group consisting of a nerve cell, cartilage cell, bone cell, muscle cell, bone cell, fat cell, or epidermal cell.
10) The cell of any of the preceding paragraphs, wherein the cell was previously differentiated in vitro into a cell selected from the group consisting of a nerve cell, cartilage cell, bone cell, muscle cell, bone cell, fat cell, or epidermal cell.
11) The cell of any of the preceding paragraphs, wherein the cell does not express an endogenous homologue of the at least one gene.
12) The cell of any of the preceding paragraphs, wherein the cell is edited to inhibit expression of an endogenous homologue of the at least one gene.
13) The cell of any of the preceding paragraphs, wherein the cell is a non-human cell.
14) The cell of any of the preceding paragraphs, wherein the cell is an elephant cell.
15) The cell of any of the preceding paragraphs, wherein the elephant cell is an African elephant (Loxodanta Africanus) cell or an Asian elephant (Elephas maximus) cell.
16) The cell of any of the preceding paragraphs, wherein the cell is a hyrax cell or manatee cell.
17) The cell of any of the preceding paragraphs, wherein the hyrax cell is selected from the group consisting of: Dendrohyrax arboreus cell, a Dendrohyrax dorsalis cell, a Heterohyrax brucei cell, and a Procavia capensis cell.
18) The cell of any of the preceding paragraphs, wherein the manatee cell is selected from the group consisting of: a Trichechus inunguis cell, a Trichechus manatus cell, a Trichechus manatus latirostris cell, a Trichechus manatus manatus cell, and a Trichechus senegalensis cell.
19) The cell of any of the preceding paragraphs, wherein the cell is cryopreserved.
20) The cell of any of the preceding paragraphs, wherein the cell was previously cryopreserved.
21) The cell of any of the preceding paragraphs, wherein the cells exhibit one or more phenotypes selected from the group consisting of: a modulation of calcium signals; a modulation of electrophysiological function; a modulation in the rate of protein synthesis, a modulation in metabolic function; and a modulation in the lipid content of the cell membrane as compared to an appropriate control.
22) An oocyte in which the endogenous nucleus has been replaced by the nucleus of a cell as described in any of the preceding paragraphs.
23) A non-wooly mammoth cell comprising at least one exogenous nucleic acid sequence selected from the group consisting of: the woolly mammoth genes in TABLE 1.
24) A gene-edited elephant cell comprising at least one exogenous nucleic acid sequence selected from the group consisting of: the woolly mammoth genes in TABLE 1, wherein the elephant cell is edited to alter an elephant homologue of the at least one gene.
25) The cell of any of the preceding paragraphs, wherein the elephant cell is edited to delete or inhibit the function of at least one gene.
26) A gene-edited elephant cell having at least one gene selected from the group consisting of (1) that is edited to mimic the wooly mammoth variant of the same gene.
27) An elephant somatic cell reprogrammed to a phenotype that is morphologically stem-like and expresses at least one endogenous stem cell marker.
28) The elephant cell of any of the preceding paragraphs, wherein the stem cell marker is selected from NANOG, SSEA1, SSEA4, or TRA-1-60.
29) The elephant cell of any of the preceding paragraphs, wherein the cell comprises exogenous nucleic acid encoding one or more exogenous polypeptide(s) selected from the group consisting of woolly mammoth polypeptides.
30) The elephant cell of any of the preceding paragraphs, wherein the elephant homologue gene(s) corresponding to the one or more exogenous polypeptide(s) is/are inactivated.
31) A non-human organism comprising the cell of any of the preceding paragraphs.
32) A non-human embryo comprising the cell of any of the preceding paragraphs.
33) A non-human embryo comprising at least one exogenous nucleic acid sequence selected from the group consisting of: the woolly mammoth genes listed in TABLE 1.
34) A non-human oocyte comprising at least one exogenous nucleic acid sequence selected from the group consisting of: the woolly mammoth genes listed in TABLE 1.
35) A non-human 4-cell stage embryo comprising at least one exogenous nucleic acid sequence selected from the group consisting of: the woolly mammoth genes listed in TABLE 1.
36) A non-human 8-cell stage embryo comprising at least one exogenous nucleic acid sequence selected from the group consisting of: the woolly mammoth genes listed in TABLE 1.
37) A non-human blastula comprising at least one exogenous nucleic acid sequence selected from the group consisting of: the woolly mammoth genes listed in TABLE 1.
38) An enucleated non-human oocyte comprising a donor nucleus comprising the nucleic acid sequence of at least at least one gene selected from the group consisting of: the woolly mammoth genes listed in TABLE 1.
39) The embryo of any of the preceding paragraphs, wherein the embryo is a pre-gastrulation embryo.
40) The embryo of any of the preceding paragraphs, wherein the embryo is a chimeric embryo.
41) The embryo, blastula, or oocyte of any of the preceding paragraphs, wherein the embryo, blastula, or oocyte is cryopreserved.
42) The oocyte, embryo or blastula of any of the preceding paragraphs, wherein the non-woolly mammoth homologue of the exogenous nucleic acid sequence has been deleted or inactivated.
43) A non-human organism comprising the nucleic acid sequence of at least one gene selected from the group consisting of: the woolly mammoth genes in TABLE 1.
44) An elephant cell comprising at least one guide RNA listed in TABLES 2 or 3.
45) The elephant cell of paragraph 44, further expressing an RNA-guided endonuclease guided by the at least one guide RNA.
46) A non-human cell comprising at least one guide RNA listed in TABLES 2 or 3.
47) The non-human cell of paragraph 46, further expressing an RNA-guided endonuclease guided by the at least one guide RNA.
48) A guide RNA comprising a sequence selected from SEQ ID NO: 1 to SEQ ID NO: 426.
49) A nucleic acid encoding a guide RNA of paragraph 48.
50) The nucleic acid of paragraph 49, wherein the nucleic acid encoding the guide RNA is operably linked to a nucleic acid sequence directing the expression of the guide RNA.
51) A vector comprising a nucleic acid of paragraph 49 or 50.
52) A cell comprising a guide RNA of paragraph 48.
53) A cell comprising a nucleic acid of paragraph 49 or paragraph 50.
54) A cell comprising a vector of paragraph 51.
55) The cell of any one of paragraphs 52-54, further comprising an RNA-guided endonuclease, the activity of which is guided by the guide RNA.

### EXAMPLES

The following examples are provided by way of illustration, not limitation.

### Example 1: Cold Adaptations of the Woolly Mammoth

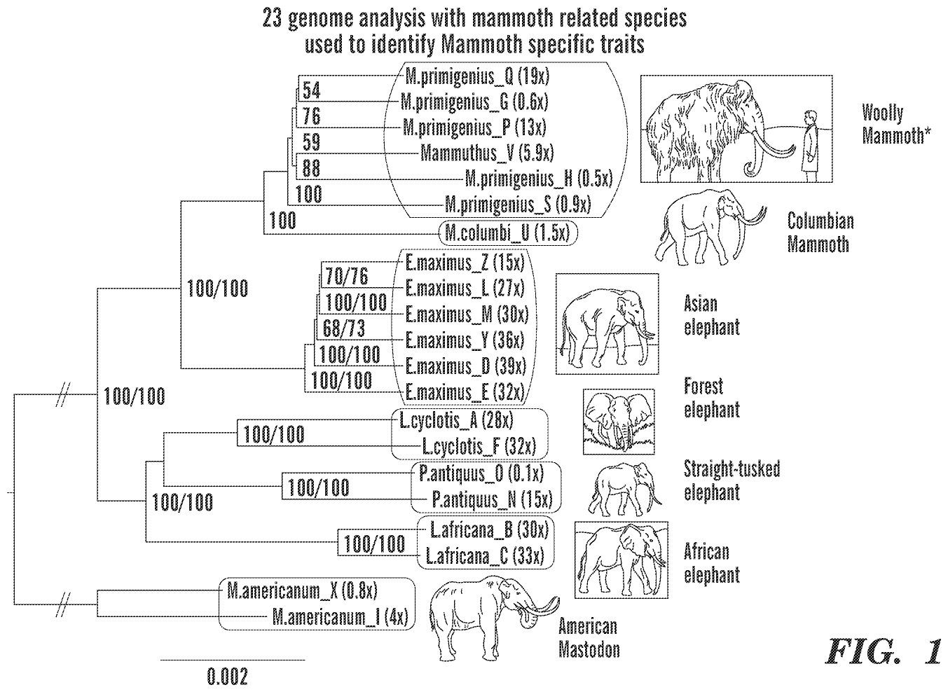

Woolly mammoths (Mammuthus primigenius) were cold-tolerant members of the elephant family that once ranged across the vast mammoth steppe of the Northern Hemisphere in the last ice age, and became extinct across the majority of their range 10,000 years ago. The woolly mammoth is arguably the best-characterized prehistoric animal, both through prehistoric art and from frozen remains found in Siberia and Alaska (FIG. 1). These well-preserved specimens provide the rare opportunity to functionally characterize adaptive evolution in an extinct animal. Inhabitation of extreme environments, such as the cold regions of the northern latitudes, necessitates a suite of adaptive evolutionary changes. Genetic and morphological analyses of woolly mammoth specimens have revealed multiple physiological adaptations to cold, including dense, long hair, increased adipose tissue, decreased ears and tails, and hemoglobin structural polymorphisms. Studies of other cold-tolerant mammals have identified a number of convergent adaptations across the same genes and pathways, as well as unique adaptations to a shared environmental stressor.

### Decreased Cold Sensitivity

The sensitivity to temperature is regulated by a series of temperature sensing ion channels in the somatosensory neurons. Polymorphisms in several of these genes (TRPM8, TRPV3, TRPA1, and TRPV4) have been identified in the woolly mammoth (Lynch et al. “Elephantid Genomes Reveal the Molecular Bases of Woolly Mammoth Adaptations to the Arctic.” Cell Reports. 12:2, p21′7-228, (2015)). Additionally, a study of the cold-tolerant thirteen-lined ground squirrel has experimentally demonstrated that the cold-insensitive TRPM8 protein, expressed in the somatosensory neurons of this species, is due to six genetic polymorphisms (Matos-Cruz et al., “Molecular Prerequisites for Diminished Cold Sensitivity in Ground Squirrels and Hamsters.” Cell Reports. 21:12, p3329-333′7, (2017)).

### Skin and Hair Development

Woolly mammoths had a number of well characterized physiological differences in their skin and hair development compared to their mid-latitude elephant relatives. Examinations of woolly mammoth hair has identified three distinct hair types, including a dense underfur that is absent in the Asian and African elephants. Examinations of well-preserved mammoth skin have also shown the presence of sebaceous glands, not present in the Asian or African elephants, which are necessary for repelling water and improving insulation. Gene ontology analyses have identified genetic polymorphisms linked to these traits in the woolly mammoth including (Lynch et al., Cell Reports. (2015)): substitutions in three genes leading to enlarged sebaceous glands (Barx2, Cd109, Rbl1), and hair development genes linked to hair root sheath development (Rbl1, Mki67, Barx2, Bnc1, Pof1b, Frem1, Bmp2, Prdm1), hair follicle (Nes, Rbl1, Dil1, Ptch1, Mki67, Sema5a, Barx2, Bnc1, Bhlhe22, Glmn, Ackr4, Frem1, Akt1, Bmp2, Selenop, Krt8, Lgals3, Ncam1, Prdm1), and hair outer root sheath (Rbl1, Mki67, Barx2, Bnc1, Frem1, Bmp2).

### Adipose Development and Lipid Metabolism

Examinations of well-preserved woolly mammoth specimens have revealed the presence of large brown-fat deposits behind the neck that are believed to have functioned as a heat source and fat reservoir during the winter (Boeskorov, G. G., Tikhonov, A. N. & Lazarev, P. A. A new find of a mammoth calf. Dokl Biol Sci 417, 480-483 (2007)). Gene ontology analyses have identified genetic polymorphisms linked to abnormal brown adipose tissue morphology (Adrb2, Dlk1, Ghr, Gpd2, Hrh1, Lepr, Lgalsl2, Lpin1, Med13, Mlxip1, Pds5b, Ptprs, Sik3, Sqstm1, ITPRID2) and abnormal brown adipose tissue amount (Dlk1, Ghr, Gpd2, Hrh1, Lepr, Lgals12, Lpin1, Med13, Mlxip1, Pds5b, Sik3, ITPRID2) in the woolly mammoth (Lynch et al., Cell Reports. (2015)). Additionally, evolutionary analyses of cold-tolerance in the mammoth revealed a statistically significant enrichment of LOF genes related to abnormal circulating lipid and cholesterol levels (Abcg8, Crp, Fabp2) (Lynch et al., Cell Reports. (2015)). Finally, altered lipid metabolism was also identified in genomic analyses of the polar bear (APOB).

### Morphological Traits

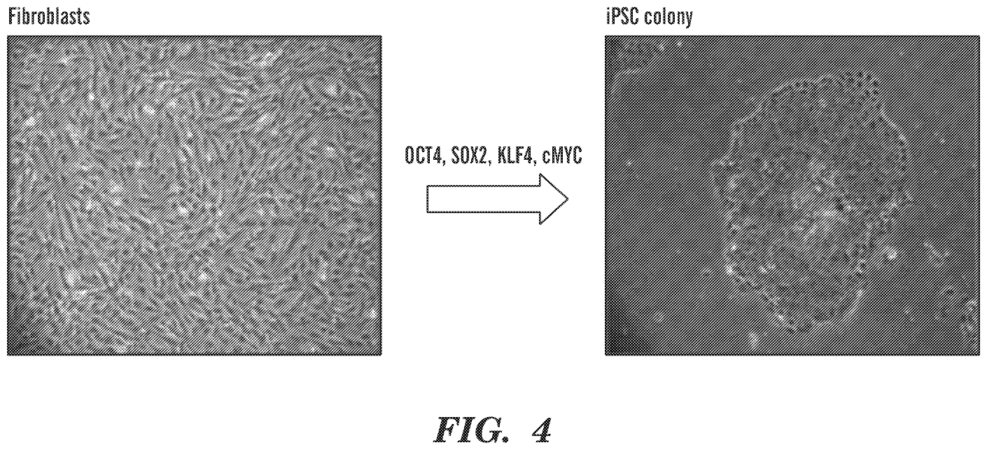

Well-preserved woolly mammoth specimens have revealed a number morphological adaptations to the cold, including smaller ears and tails, shorter trunks, and domed craniums. Gene ontology analyses have identified genetic polymorphisms linked to these traits in the woolly mammoth including: abnormal tail morphology (Apaf1, Avil, Axin2, Bmp2, Brca1, Brca2, Cdc7, Celsr1, Chst14, Crh, Dact1, Dil1, Dmrt2, Dst, Fat4, Fn1, Hist1h1c, Jak1, Krt76, Lepr, Lrp2, Lyst, Med12, Mthfr, Ndc1, Noto, Phc1, Phc2, Ptch1, Rc3h1, Sepp1, Slx4, Sytl1, Tcea1, Zeb1), abnormal tail bud morphology (Brca1, Dact1, Fn1, Phc1, Phc2), small tail bud (Phc1, Phc2), abnormal ear morphology (Apaf1, Atp8b1, Bhlhe22, Bmp2, Celsr1, Col9a1, Dil1, Fat4, Foxq1, Gpr98, Htt, Jag1, Jak1, Loxhd1, Lrp2, Lyst, Mecom, Muc5b, Nf1, Otoa, Pcdh15, Phc1, Phc2, Pqvq, Synj2, Tbx10, Tcof1, Tub, Zeb1), cup-shaped ears (Tcof1), domed cranium (Col27a1, FIG. 4, Hdac4, Htt, Pfas, Pkd1, Ptch1, Slx4, Tcof1, Trip1), abnormal parietal bone morphology (Apaf1, Hhat, Neil1, Ptch1, Sik3, Tcof1), and a short snout (Apaf1, Asph, Col27a1, Frem1, Hhat, Kif20b, Lrp2, Ltbp1, Mia3, Pds5b, Pfas, Pkd1, Rbl1, Trip11, Zc3hc1).

### Blood Adaptations

Hemoglobin is a temperature-sensitive tetrameric protein that binds oxygen in the blood. At cold temperatures, oxygen molecules cannot be offloaded to the tissues. Wooly mammoth substitutions in the hemoglobin alpha and beta genes (HBA, HBB) have been experimentally shown to improve oxygen delivery at cold temperatures (Campbell, K., Roberts, J., Watson, L. et al. Substitutions in woolly mammoth hemoglobin confer biochemical properties adaptive for cold tolerance. Nat Genet 42, 536-540 (2010)). The platelets of non-cold-tolerant mammals develop lesions upon exposure to cold. In contrast, platelets in the thirteen-lined ground squirrel have been experimentally shown to be resistant to these lesions (Cooper et al., The hibernating 13-lined ground squirrel as a model organism for potential cold storage of platelets. American Journal of Physiology-Regulatory, Integrative and Comparative Physiology (2012)).

### Circadian Biology

Clock genes play key roles in timing certain cellular and metabolic events. In arctic animals, which experience prolonged periods of darkness or daylight, loss of function (LOF) mutations have been identified in several of the key circadian clock genes. Notably, reindeer do not exhibit circadian melatonin rhythms and reindeer fibroblasts grown in culture lack the typical rhythmic clock gene activity. It has been suggested that these observed phenotypes are due to LOF mutations in Per2 and Bmal1. Similarly, in the woolly mammoth, LOF mutations in the following clock genes have been identified: Hrh3, Lepr, Per2 (Lynch et al. Cell Reports. (2015)).

### Example 2: Adaptive Genes that Confer Decreased Cold Sensitivity in the Woolly Mammoth and Other Cold-Climate Wildlife

The following genes were discovered to be important for the adaptations of the woolly mammoth and other animals (e.g., reindeer and polar bears) to colder climates.

| Gene | Adaptive phenotype | Species |
|---|---|---|
| TRPM8 | Decreased cold sensitivity | mammoth |
| TRPV3 | Decreased cold sensitivity | mammoth |
| TRPA1 | Decreased cold sensitivity | mammoth |
| TRPV4 | Decreased cold sensitivity | mammoth |
| PER2 | Circadian biology | reindeer, mammoth |
| BMAL1 | Circadian biology | reindeer, mammoth |
| HRH3 | Circadian biology | mammoth |
| LEPR | Circadian biology | mammoth |
| CD109 | Sebaceous glands | mammoth |
| BARX2 | Sebaceous glands & Hair | mammoth |
| RBL1 | Sebaceous glands & Hair | mammoth |
| MKI67 | Hair development | mammoth |
| BNC1 | Hair development | mammoth |
| POF1B | Hair development | mammoth |
| FREM1 | Hair development | mammoth |
| BMP2 | Hair development | mammoth |
| PRDM1 | Hair development | mammoth |
| NES | Hair development | mammoth |
| DLL1 | Hair development | mammoth |
| PTCH1 | Hair development | mammoth |
| SEMA5A | Hair development | mammoth |
| BNC1 | Hair development | mammoth |
| BHLHE22 | Hair development | mammoth |
| GLMN | Hair development | mammoth |
| ACKR4 | Hair development | mammoth |
| AKT1 | Hair development | mammoth |
| SELENOP | Hair development | mammoth |
| KRT8 | Hair development | mammoth |
| NCAM1 | Hair development | mammoth |
| APOB | Lipid metabolism | polar bear |
| ABCG8 | Lipid metabolism | polar bear |
| CRP | Lipid metabolism | polar bear |
| FABP2 | Lipid metabolism | polar bear |
| UCP1 | Brown fat | mouse |
| DLK1 | Brown fat | mammoth |
| GHR | Brown fat | mammoth |
| GPD2 | Brown fat | mammoth |
| HRH1 | Brown fat | mammoth |
| LEPR | Brown fat | mammoth |
| LGALS12 | Brown fat | mammoth |
| LPIN1 | Brown fat | mammoth |
| MED13 | Brown fat | mammoth |
| MLXIPL | Brown fat | mammoth |
| PDS5B | Brown fat | mammoth |
| SIK3 | Brown fat | mammoth |
| ITPRID2 | Brown fat | mammoth |
| COL27A1 | Domed cranium | mammoth |
| FIG4 | Domed cranium | mammoth |
| HDAC4 | Domed cranium | mammoth |
| HTT | Domed cranium | mammoth |
| PFAS | Domed cranium | mammoth |
| PKD1 | Domed cranium | mammoth |
| SLX4 | Domed cranium | mammoth |
| TCOF1 | Domed cranium | mammoth |
| TRIP1 | Domed cranium | mammoth |
| PHC1 | Small tail bud | mammoth |
| PHC2 | Small tail bud | mammoth |
| FN1 | Small tail bud | mammoth |
| DACT1 | Small tail bud | mammoth |
| HBB | Oxygen delivery | mammoth |
| HBA | Oxygen delivery | mammoth |

### Example 3: Additional Examples of Genes that Confer Decreased Cold-Climate Sensitivity

HBB (hemoglobin (3/6 fusion gene): amino acid polymorphism in the woolly mammoth HBB reduces oxygen affinity. Mutations in this gene subunit decrease the energetic cost of delivering oxygen from lungs.
HBA-2 (variant of hemoglobin subunit A)
Temperature-sensitive transient receptor potential (thermoTRP)
   TRPA1—sense noxious cold or heat depending on species
   TRPV3—sense innocuous warmth. A mammoth-specific substitution in TRPV3 (N647D) occurred at a well-conserved site that may affect thermosensation by mammoth TRPV3. Associates with evolution of cold tolerance, long hair, and large adipose stores in mammoths.
   TRPM4—it is heat sensitive but not known to be involved in temperature sensation—
TRPM8—sense noxious cold

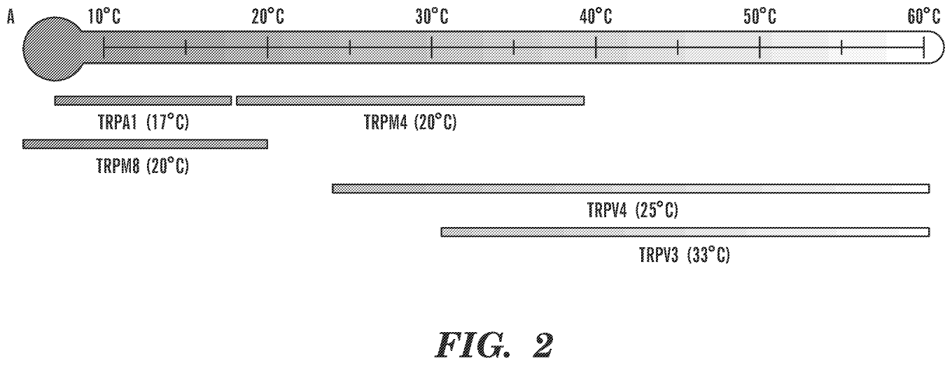

FIG. 2 shows temperature ranges over which TRP genes are active.

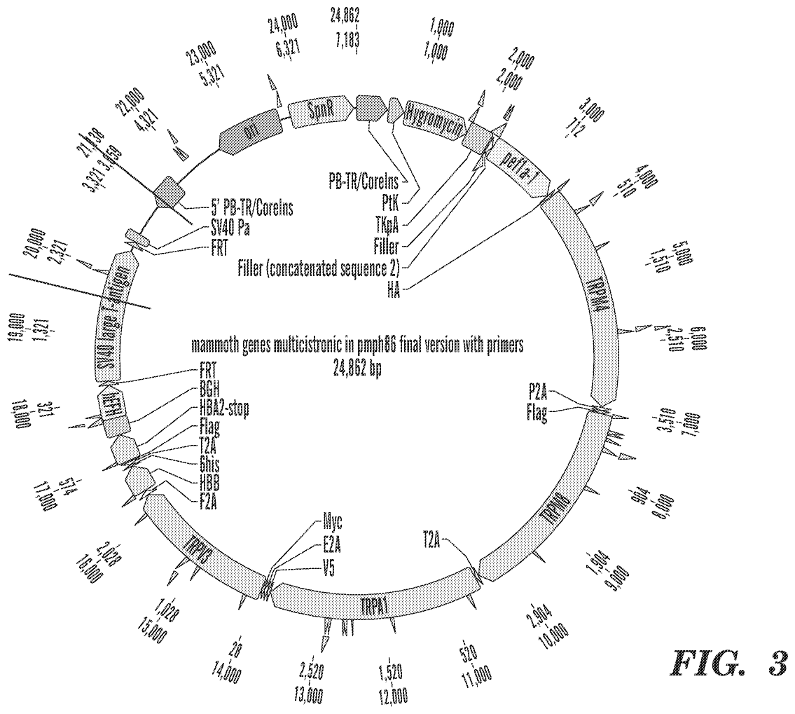

FIG. 3 shows a multicistronic vector with cloned mammoth alleles.

### Example 4: Generation of a Multicistronic Vector and Reprogramming of African Elephant Cells

A multicistronic vector with cloned mammoth alleles was generated (FIG. 3-5).

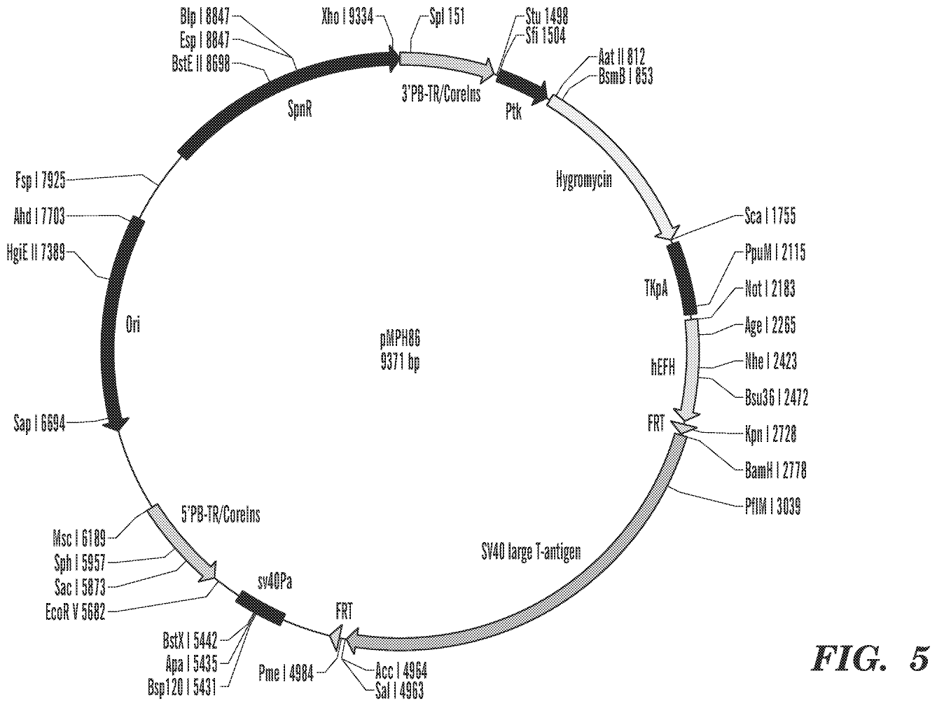

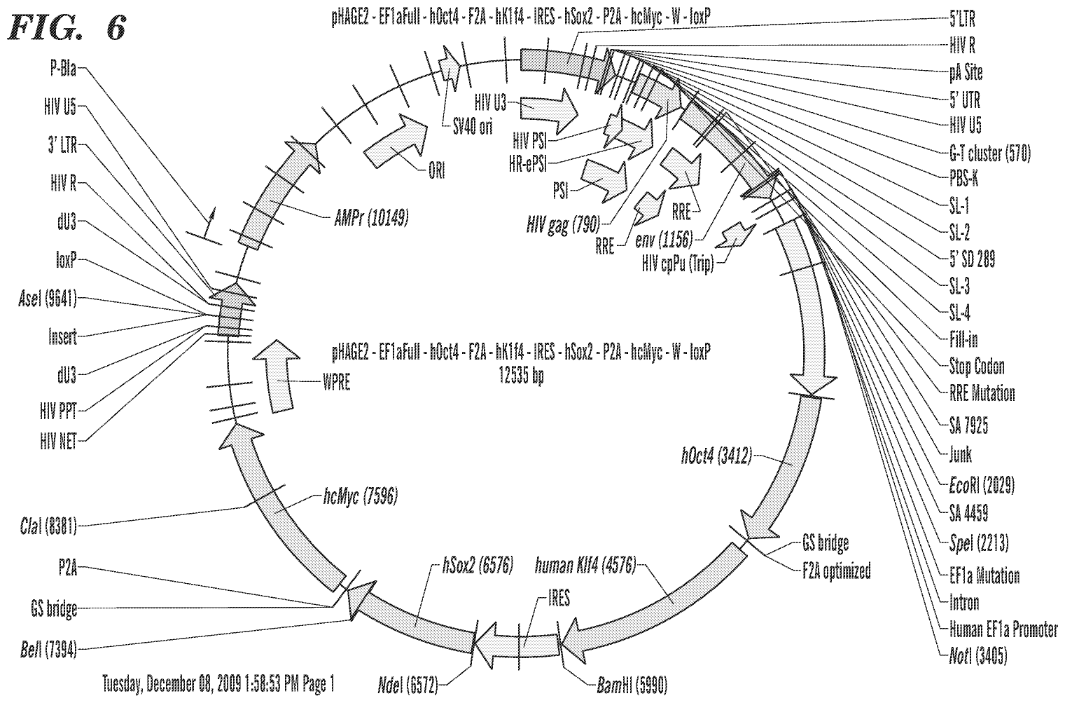

Next, induced stem cells from a biopsy of an African elephant (Loxodonta africana) frozen placenta were obtained and maintained in culture (FIG. 4, left). A transposon plasmid was generated containing SV40LT and hygromycin resistance genes. The plasmid was generated by cloning pHAGE2-EF1-OSKM into a Pme1 site that contains the human reprogramming factors OCT4, SOX2, KLF4, and c-MYC, immortalization gene SV40LT, and a hygromycin selectable marker (FIGS. 5-6).

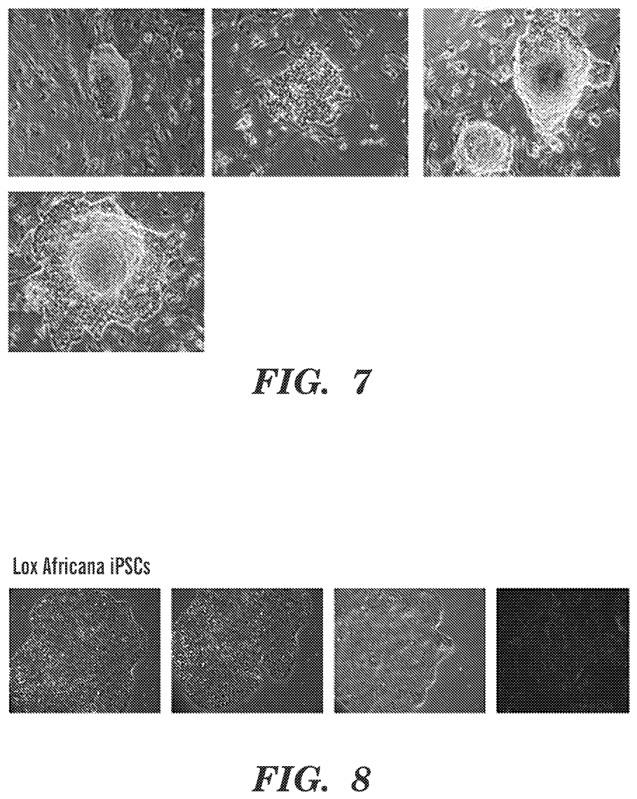

Loxodonta africana cells were transfected with the transposon reprogramming factors and transposase. Cells were selected in the presence of hygromycin and surviving cells were expanded and reprogramming was initiated with the reprogramming vectors described above (FIGS. 3-6). Cell colonies were derived in a layer of feeder cells (MEFs) (plate pre-coated with 0.1% gelatin) and maintained in a medium referred to herein as Essential 8 (Gibco) that contains a proprietary formulation with insulin, selenium, transferrin, L-ascorbic acid, FGF2, and TGFβ (or NODAL) in DMEM/F12 with pH adjusted with NaHCO₃ (e.g., as described in Chen G, et al. Nat Methods. 2011). (FIG. 7). Colonies started to emerge at two weeks. Single colonies were transferred to matrigel-coated plates and maintained in feeder-free conditions with Essential 8.

Loxodonta africana induced stem cell colonies were then expanded in feeder-free conditions with MATRIGEL™ (FIG. 8). In order to test differentiation into different lineages, a teratoma assay was performed. The Loxodonta africana induced stem cells were injected into immune-compromised mice.

Cells can be differentiated along different lineages via various protocols known in the art from induced stem cell stage, or transdifferentiated with distinct transcription factors from fibroblast-like to other cell types.

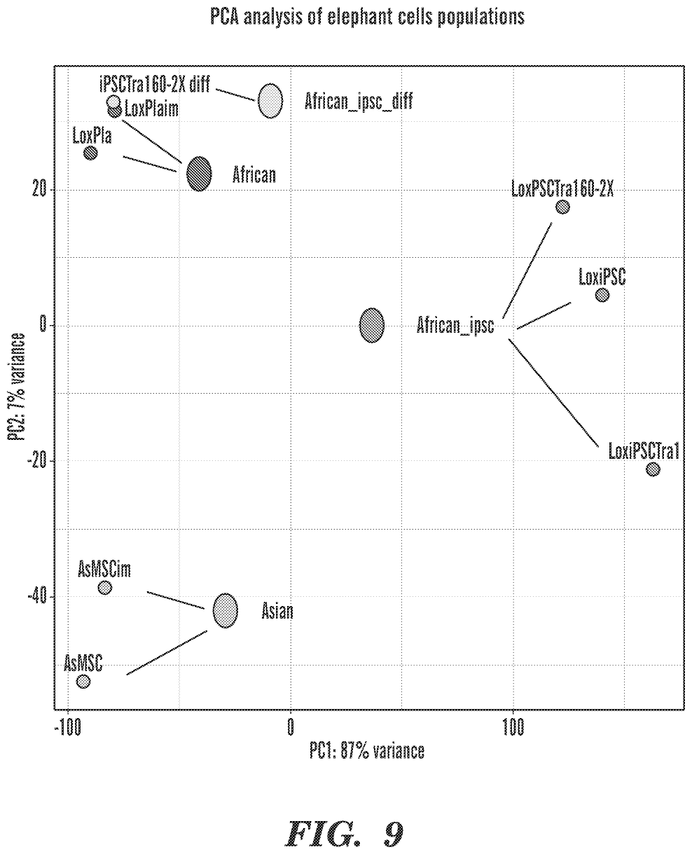

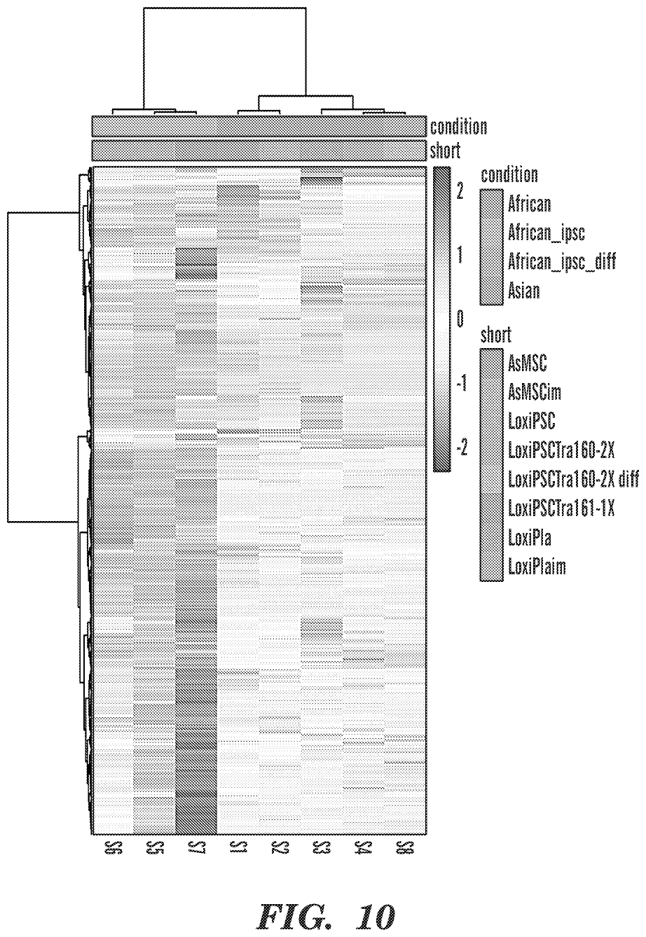

RNA seq experiments of the Loxodonta africana induced stem cell populations demonstrated that the cells are closer to a pluripotent cell than to a terminally differentiated phenotype. Principal Component Analysis, or PCA was used to identify specific properties of the following cells:
ele1 AsMSC Af28 Asian Mesenchymal stem cells (Asian elephant parental cells);
ele2 AsMSCim Af28 Asian Mesenchymal stem cells SV40LT (Asian elephant parental cells immortalized);
ele3 LoxPla Loxodonta Afr Placental cells P.3 (African elephant parental cells);
ele4 LoxPlaim Loxodonta Afr Placental cells SV40LT (African elephant parental cells immortalized);
ele5 LoxiPSC P.9 induced stem cells from Loxodonta placenta (African elephant induced stem cells);
ele6 LoxiPSCTra160-2× sorted 2× with TRA160 PE and FITC P.7 (African elephant induced stem cells sorted);
ele7 LoxiPSCTra161-1× sorted 1× with TRA160 FITC P.9 (African elephant induced stem cells sorted); and
ele8 LoxiPSCTra160-2× diff sorted 2× with TRA160 PE and FITC P.7 differentiated (African elephant differentiated from stem cells) (FIG. 9).

A heatmap of the various Loxodonta africana induced stem cell populations was constructed to determine which pluripotent cell markers were prominently expressed in the elephant induced stem cells and low in the fibroblast-like cells obtained from Loxodonta africana (FIG. 10).

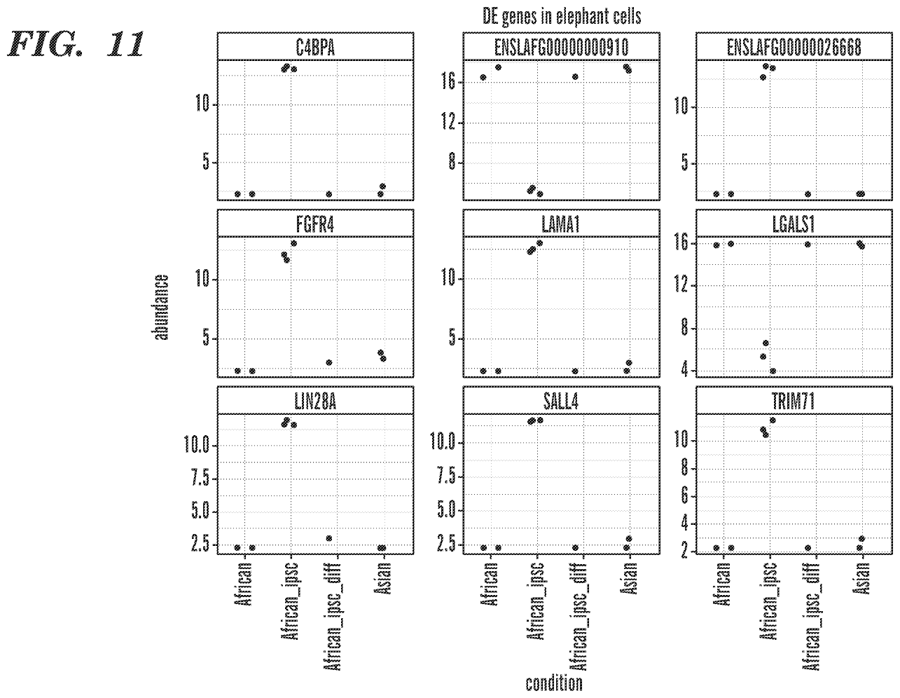

A computational comparison of differentiation markers that were low in elephant induced stem cells and high in differentiated parental cell populations was performed. Genes that were differentially expressed in the elephant cells included LIN 28A, SALL4, TRIM 7, LAMA1, ENSLAFG00000026668, FGFR4, and C4BPA with increased abundance in Loxodonta africana induced stem cells and ENSSLAFG00000000910, LGALS1 with decreased abundance in Loxodonta africana induced stem cells (FIG. 11).

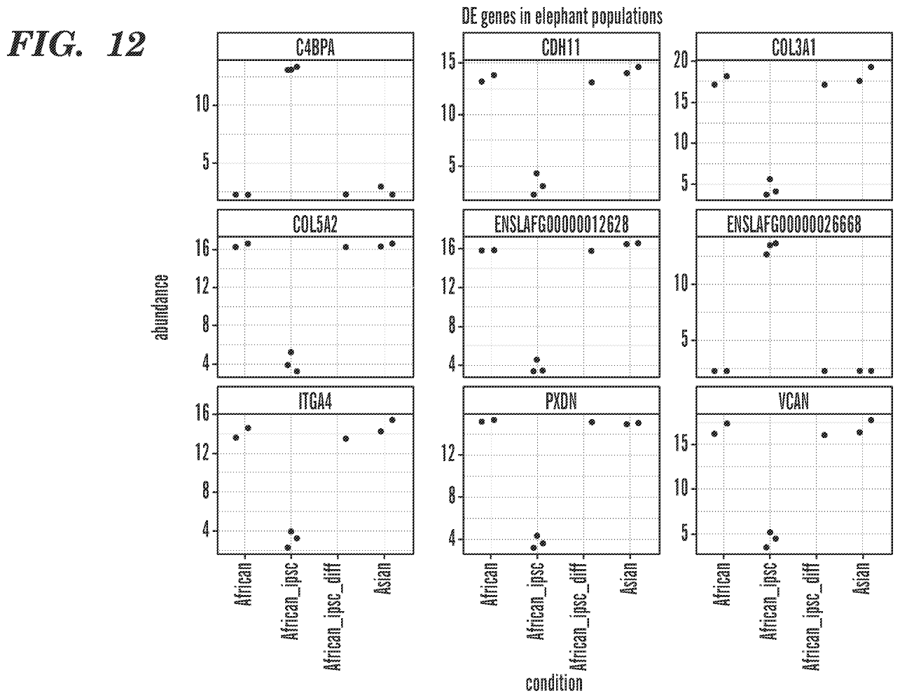

In addition, about 11,000 SNP changes in coding regions of the genes differentially expressed in the Loxodonta africana induced stem cell populations were observed. Many ENSLAF genes were annotated and have unknown functional effects. Gene ontology analysis revealed that the genes that are enriched in this analysis are correlated with developmental, cell cycle, ion channels, and metabolism pathways (FIG. 12).

A 23 genome analysis with mammoth related species was used to identify mammoth specific traits (FIG. 1). The genes are involved in several biological processes, molecular functions, and classes of proteins listed in the table below.

Biological Processes
Regulation of intracellulular pH
regulation of axonogenesis/developmental process
tRNA/metabolic processes
cell-cell adhesion
tissue development
microtubule-based movement
negative regulation of biological process
gene expression
cellular macromolecule metabolic process
Molecular Function
Tyrosine kinase
calcium channel activity
sodium ion transmembrane transporter activity
secondary active transmembrane transporter activity
active transmembrane transporter activity
active ion transmembrane transporter activity
catalytic activity, acting on RNA
phosphoric ester hydrolase activity
hydrolase activity, acting on ester bonds
cytoskeletal protein binding
ATPase activity
Unclassified
Protein classes
metalloprotease
protein modifying enzyme
ion channel
transporter
G-protein modulator
hydrolase
metabolite interconversion enzyme
transferase
nucleic acid binding protein
Unclassified
immunoglobulin receptor superfamily
defense/immunity protein
immunoglobulin

TABLE 2

| | | | | | | | | | Engi- | | | | |
|---|---|---|---|---|---|---|---|---|---|---|---|---|---|
| | | | Engi- | | | | | | neer- | | | | |
| | | | neer- | | | | | | ing | | | | |
| | | | ing | | | | | | Tool | | | | |
| Afri- | | | Tool | | | | | | option | | | | |
| can | Wool- | | option | | | | | | 2 | | | | |
| Ele- | ly | Amino | 1 | Edit- | | SEQ | | SEQ | (SpCas9) | | SEQ | | SEQ |
| phant | Mam- | Acid | (SPRYC) | ing | | ID | | ID | Editing | | ID | | ID |
| Ref | moth | Change | Gene | Method | sgRNA | NO: | PAM | NO: | Method | sgRNA | NO: | PAM | NO: |
| T | A | p.Thr470Ser | KRT8 | HDR | ACCACA | 1 | GGTA | 54 | HDR | ACCACA | 106 | GGTA | 158 |
| | | | | | GTCTTG | | | | | GTCTTG | | | |
| | | | | | GTGGAG | | | | | GTGGAG | | | |
| | | | | | CCG | | | | | CCG | | | |
| C | T | p.Gly454Ser | KRT8 | CBE | CCAGAG | 2 | GAAG | 55 | CBE | CAGAGC | 107 | AAGG | 159 |
| | | | | | CCGAAG | | | | | CGAAGC | | | |
| | | | | | CTAGAC | | | | | TAGACT | | | |
| | | | | | TGG | | | | | GGA | | | |
| T | C | p.Gln357Arg | KRT8 | ABE | GATGCC | 3 | TGAG | 56 | ABE | GATGCC | 108 | TGAG | 160 |
| | | | | | CAAAAC | | | | | CAAAAC | | | |
| | | | | | AAGCTG | | | | | AAGCTG | | | |
| | | | | | GCT | | | | | GCT | | | |
| C | A | p.Ala340Ser | KRT8 | HDR | GCAGCC | 4 | CTGT | 57 | HDR | GGCAGC | 109 | TCTG | 161 |
| | | | | | TCCAGG | | | | | CTCCAG | | | |
| | | | | | GAAGCC | | | | | GGAAGC | | | |
| | | | | | CTC | | | | | CCT | | | |
| C | G | p.Glu339Asp | KRT8 | HDR | GCAGCC | 5 | CTGT | 58 | HDR | GGCAGC | 111 | TCTG | 162 |
| | | | | | TCCAGG | | | | | CTCCAG | | | |
| | | | | | GAAGCC | | | | | GGAAGC | | | |
| | | | | | CTC | | | | | CCT | | | |
| T | G | p.Lys312Gln | KRT8 | HDR | TCAGTC | 6 | GTCA | 59 | HDR | GTCTTC | 112 | ATCC | 163 |
| | | | | | TTCGTA | | | | | GTACGA | | | |
| | | | | | CGACGA | | | | | CGAAGG | | | |
| | | | | | AGG | | | | | TCA | | | |
| C | T | p.Arg310His | KRT8 | CBE | CGTACG | 7 | CGTG | 60 | CBE | CGTACG | 113 | CGTG | 164 |
| | | | | | ACGAAG | | | | | ACGAAG | | | |
| | | | | | GTCATC | | | | | GTCATC | | | |
| | | | | | CCC | | | | | CCC | | | |
| C | T | p.Ala245Thr | KRT8 | CBE | ATCTGG | 8 | GATC | 61 | CBE | CTGGGC | 114 | TCTC | 165 |
| | | | | | GCCTGC | | | | | CTGCAG | | | |
| | | | | | AGCTCA | | | | | CTCACG | | | |
| | | | | | CGG | | | | | GAT | | | |
| G | A | p.Ser35Phe | KRT8 | CBE | TCAGCT | 9 | CGGG | 62 | CBE | ATCAGC | 115 | CCGG | 166 |
| | | | | | CTTCTG | | | | | TCTTCT | | | |
| | | | | | CCTTCT | | | | | GCCTTC | | | |
| | | | | | CCC | | | | | TCC | | | |
| C | G | p.Gly28Ala | KRT8 | HDR | GCCGGG | 10 | AGCG | 63 | HDR | GCCGGG | 116 | AGCG | 167 |
| | | | | | CCCGCT | | | | | CCCGCT | | | |
| | | | | | CGTGTA | | | | | CGTGTA | | | |
| | | | | | AGA | | | | | AGA | | | |
| A | T | p.Cys711Ser | TRPM8 | HDR | GCCACA | 11 | TAAT | 64 | HDR | CCACAG | 117 | AATA | 168 |
| | | | | | GCCGAC | | | | | CCGACC | | | |
| | | | | | CAAAGG | | | | | AAAGGT | | | |
| | | | | | TAT | | | | | ATA | | | |
| C | T | p.Gly710Ser | TRPM8 | CBE | CCACAG | 12 | AATA | 65 | CBE | CCACAG | 118 | AATA | 169 |
| | | | | | CCGACC | | | | | CCGACC | | | |
| | | | | | AAAGGT | | | | | AAAGGT | | | |
| | | | | | ATA | | | | | ATA | | | |
| C | A | p.Ala533Ser | TRPM8 | HDR | AAGTTT | 13 | AACA | 66 | HDR | AGTTTG | 119 | ACAA | 170 |
| | | | | | GCGACC | | | | | CGACCA | | | |
| | | | | | AGCTTC | | | | | GCTTCC | | | |
| | | | | | CAA | | | | | AAA | | | |
| C | T | p.Arg368His | TRPM8 | CBE | CACCGT | 14 | GCAC | 67 | CBE | CACCGT | 120 | GCAC | 171 |
| | | | | | ACGGGG | | | | | ACGGGG | | | |
| | | | | | CAGAAA | | | | | CAGAAA | | | |
| | | | | | GCG | | | | | GCG | | | |
| A | C | p.Leu107Arg | TRPV3 | HDR | CTTGGC | 15 | GGCC | 68 | HDR | CTTGGC | 121 | GGCC | 172 |
| | | | | | CAGGTT | | | | | CAGGTT | | | |
| | | | | | TGCACT | | | | | TGCACT | | | |
| | | | | | GAG | | | | | GAG | | | |
| C | A | p.Gly1016Val | TRPA1 | HDR | CATTAG | 16 | TGGG | 69 | HDR | CATTAG | 122 | TGGG | 173 |
| | | | | | CCCCCC | | | | | CCCCCC | | | |
| | | | | | TTGGTA | | | | | TTGGTA | | | |
| | | | | | TCT | | | | | TCT | | | |
| A | T | p.Asn614His | PER2 | HDR | GGCCCT | 17 | AGCG | 70 | HDR | GGCCCT | 123 | AGCG | 174 |
| | | | | | GAATGC | | | | | GAATGC | | | |
| | | | | | CAGCGA | | | | | CAGCGA | | | |
| | | | | | CAA | | | | | CAA | | | |
| A | stop | p.Asn614* | PER2 | HDR | GGCCCT | 18 | AGCG | 71 | HDR | GGCCCT | 124 | AGCG | 175 |
| | | | | | GAATGC | | | | | GAATGC | | | |
| | | | | | CAGCGA | | | | | CAGCGA | | | |
| | | | | | CAA | | | | | CAA | | | |
| A | G | p.Phe786Leu | LEPR | ABE | TCCTGA | 19 | AGTG | 72 | ABE | TCCTGA | 125 | AGTG | 176 |
| | | | | | AAAATC | | | | | AAAATC | | | |
| | | | | | CTGATG | | | | | CTGATG | | | |
| | | | | | TCA | | | | | TCA | | | |
| G | A | p.Pro838Ser | CD109 | CBE | CGTTTC | 20 | ATGC | 73 | CBE | CGTTTC | 126 | ATGC | 177 |
| | | | | | ACCTAC | | | | | ACCTAC | | | |
| | | | | | TGCTTC | | | | | TGCTTC | | | |
| | | | | | TGA | | | | | TGA | | | |
| G | C | p.Gln804Glu | CD109 | HDR | TGCTGG | 21 | TACT | 74 | HDR | GTCTGC | 127 | GTTT | 178 |
| | | | | | TATCCT | | | | | TGGTAT | | | |
| | | | | | GTTGCG | | | | | CCTGTT | | | |
| | | | | | TTT | | | | | GCG | | | |
| T | C | p.Asn294Asp | CD109 | ABE | CTCTTT | 22 | TGAA | 75 | ABE | CTCTTT | 128 | TGAA | 179 |
| | | | | | TAATGA | | | | | TAATGA | | | |
| | | | | | GGAAGA | | | | | GGAAGA | | | |
| | | | | | GAT | | | | | GAT | | | |
| C | T | p.Arg68Gln | BARX2 | CBE | ATAAGC | 23 | AACC | 76 | CBE | AGCCCG | 129 | CTGA | 180 |
| | | | | | CCGAAG | | | | | AAGGGA | | | |
| | | | | | GGATGG | | | | | TGGGGA | | | |
| | | | | | GGA | | | | | ACC | | | |
| T | C | p.Ile979Val | RBL1 | HDR | TGGGAA | 24 | GCCT | 77 | HDR | GGAAAT | 130 | CTGG | 181 |
| | | | | | ATGCGG | | | | | GCGGCG | | | |
| | | | | | CGGGGT | | | | | GGGTGA | | | |
| | | | | | GAG | | | | | GCC | | | |
| C | T | p.Gly50Ser | HBA2 | CBE | CCATGG | 25 | AGGA | 78 | | | | | |
| | | | | | CCCAGG | | | | | | | | |
| | | | | | TCGAAG | | | | | | | | |
| | | | | | TGA | | | | | | | | |
| A | G | p.Leu183Ser | BMP2 | ABE | GAATTT | 26 | CAGG | 79 | ABE | GAATTT | 131 | CAGG | 182 |
| | | | | | CAAGTT | | | | | CAAGTT | | | |
| | | | | | GGTGGG | | | | | GGTGGG | | | |
| | | | | | TGC | | | | | TGC | | | |
| C | T | p.Glu690Lys | NES | CBE | TTTTCT | 27 | CAGT | 80 | CBE | TGATTT | 132 | TCTC | 183 |
| | | | | | TTTGCT | | | | | TCTTTT | | | |
| | | | | | AGATGT | | | | | GCTAGA | | | |
| | | | | | CTC | | | | | TGT | | | |
| C | G | p.Glu625Asp | NES | HDR | CTTGAT | 28 | GATT | 81 | HDR | TGATTC | 133 | TTGC | 184 |
| | | | | | TCTCCT | | | | | TCCTTT | | | |
| | | | | | TTTCTA | | | | | TCTAGA | | | |
| | | | | | GAG | | | | | GAT | | | |
| C | T | p.Val611Ile | NES | CBE | TTCTAC | 29 | CTAG | 82 | CBE | TTTCTA | 134 | TCTA | 185 |
| | | | | | GGGTGT | | | | | CGGGTG | | | |
| | | | | | AAGTAG | | | | | TAAGTA | | | |
| | | | | | TTC | | | | | GTT | | | |
| T | A | p.Met132Lys | BHLHE22 | HDR | CGGATG | 30 | CACG | 83 | HDR | GTGCGG | 135 | CGCC | 186 |
| | | | | | CTCTCC | | | | | ATGCTC | | | |
| | | | | | AAGATC | | | | | TCCAAG | | | |
| | | | | | GCC | | | | | ATC | | | |
| C | T | p.Glu50Lys | CRP | CBE | GCCTCG | 31 | CAGT | 84 | CBE | AAGGCC | 136 | TCTC | 187 |
| | | | | | AGTGGC | | | | | TCGAGT | | | |
| | | | | | TGCTTT | | | | | GGCTGC | | | |
| | | | | | CTC | | | | | TTT | | | |
| G | A | p.Arg96* | FABP2 | CBE | ATTCAA | 32 | GAAA | 85 | CBE | TCAAGC | 137 | AAGG | 188 |
| | | | | | GCGAGT | | | | | GAGTAG | | | |
| | | | | | AGACAA | | | | | ACAATG | | | |
| | | | | | TGG | | | | | GAA | | | |
| C | T | p.Val405Met | HRH1 | CBE | CGGTTC | 33 | CACA | 86 | CBE | CGGTTC | 138 | CACA | 189 |
| | | | | | ACGTGC | | | | | ACGTGC | | | |
| | | | | | AACCCA | | | | | AACCCA | | | |
| | | | | | GAC | | | | | GAC | | | |
| G | C | p.Ser257Arg | HRH1 | HDR | TCGCTG | 34 | TGGT | 87 | HDR | ACCTCG | 139 | CCCT | 190 |
| | | | | | AAGGAC | | | | | CTGAAG | | | |
| | | | | | TCTCTC | | | | | GACTCT | | | |
| | | | | | CCT | | | | | CTC | | | |
| C | T | p.Arg311Gln | LGALS12 | CBE | ACTGAT | 35 | GCCG | 88 | CBE | ACTGAT | 140 | GCCG | 191 |
| | | | | | CCGAAG | | | | | CCGAAG | | | |
| | | | | | CTCCCG | | | | | CTCCCG | | | |
| | | | | | CAG | | | | | CAG | | | |
| G | T | p.Ser1409* | MED13 | HDR | TTTCTT | 36 | CAAC | 89 | HDR | TTTGAT | 141 | TCTC | 192 |
| | | | | | TGATGC | | | | | GCAGTA | | | |
| | | | | | AGTAGA | | | | | GATCCA | | | |
| | | | | | TCC | | | | | ACT | | | |
| G | T | p.Ser1406Tyr | MED13 | HDR | TGCAGT | 37 | TGAT | 90 | HDR | TGCAGT | 142 | TGAT | 193 |
| | | | | | AGATCC | | | | | AGATCC | | | |
| | | | | | AACTCT | | | | | AACTCT | | | |
| | | | | | CAT | | | | | CAT | | | |
| C | G | p.Pro393Ala | MLXIPL | HDR | CCACAC | 38 | CACT | 91 | HDR | | | | |
| | | | | | CCCCAC | | | | | | | | |
| | | | | | CCCTCC | | | | | | | | |
| | | | | | TCC | | | | | | | | |
| C | T | p.Gly883Ser | FIG4 | CBE | CCTTTA | 39 | GGCT | 92 | CBE | TTACCG | 143 | TTGG | 194 |
| | | | | | CCGGCC | | | | | GCCTGG | | | |
| | | | | | TGGATG | | | | | ATGTGG | | | |
| | | | | | TGG | | | | | GCT | | | |
| G | C | p.Asp874Glu | FIG4 | HDR | GGAAGA | 40 | CTGT | 93 | HDR | GGAAGA | 144 | CTGT | 195 |
| | | | | | TGTCTG | | | | | TGTCTG | | | |
| | | | | | TGGATT | | | | | TGGATT | | | |
| | | | | | TTC | | | | | TTC | | | |
| A | G | p.Thr402Ala | HDAC4 | ABE | TGGGCA | 41 | GCCC | 94 | ABE | CCTGGG | 145 | ACGC | 196 |
| | | | | | CGCTGC | | | | | CACGCT | | | |
| | | | | | CCCTCC | | | | | GCCCCT | | | |
| | | | | | ACG | | | | | CCA | | | |
| A | G | p.Thr537Ala | HDAC4 | ABE | GGAGGA | 42 | GGGA | 95 | ABE | GGAGGA | 146 | GGGA | 197 |
| | | | | | GACAGA | | | | | GACAGA | | | |
| | | | | | GGCTGC | | | | | GGCTGC | | | |
| | | | | | CCG | | | | | CCG | | | |
| T | C | p.Ile2858Val | HTT | ABE | TCGGCC | 43 | CTTG | 96 | ABE | CGGCCA | 147 | TTGC | 198 |
| | | | | | ATCTTC | | | | | TCTTCC | | | |
| | | | | | CACTGC | | | | | ACTGCG | | | |
| | | | | | GTC | | | | | TCT | | | |
| A | C | p.Asp2752Glu | HTT | HDR | CGCGCT | 44 | TGAC | 97 | HDR | GCGCGC | 148 | CTGA | 199 |
| | | | | | ATCCAG | | | | | TATCCA | | | |
| | | | | | CAGACG | | | | | GCAGAC | | | |
| | | | | | GCT | | | | | GGC | | | |
| C | T | p.Arg10Cys | PFAS | CBE | CTATGT | 45 | ATGA | 98 | CBE | TATGTC | 149 | TGAG | 200 |
| | | | | | CCGTCC | | | | | CGTCCC | | | |
| | | | | | CTCTGG | | | | | TCTGGC | | | |
| | | | | | CCA | | | | | CAT | | | |
| A | G | p.Gln1030Arg | PFAS | ABE | GTGGCA | 46 | GCTG | 99 | ABE | TGGCAC | 150 | CTGA | 201 |
| | | | | | CAGGAG | | | | | AGGAGG | | | |
| | | | | | GAAAAG | | | | | AAAAGG | | | |
| | | | | | GGG | | | | | GGC | | | |
| G | A | p.Glu1176Lys | PFAS | CBE | TCCTCG | 47 | GACC | 100 | CBE | CCTCCT | 151 | CCGA | 202 |
| | | | | | TTGGGG | | | | | CGTTGG | | | |
| | | | | | TCGCCC | | | | | GGTCGC | | | |
| | | | | | CCG | | | | | CCC | | | |
| G | A | p.Val222Met | PKD1 | CBE | GGGAGC | 48 | ACAA | 101 | CBE | GGGAGC | 152 | ACAA | 203 |
| | | | | | ACGGTG | | | | | ACGGTG | | | |
| | | | | | GGGCCC | | | | | GGGCCC | | | |
| | | | | | CCA | | | | | CCA | | | |
| T | C | p.Met505Thr | PKD1 | ABE | GCTCCC | 49 | CGCA | 102 | ABE | CGCTCC | 153 | CCGC | 204 |
| | | | | | ATGAGG | | | | | CATGAG | | | |
| | | | | | ACATTC | | | | | GACATT | | | |
| | | | | | TCC | | | | | CTC | | | |
| G | A | p.Arg750Gln | PKD1 | CBE | AGGATG | 50 | TGGG | 103 | CBE | AGGATG | 154 | TGGG | 205 |
| | | | | | TCGAAG | | | | | TCGAAG | | | |
| | | | | | CCCAGG | | | | | CCCAGG | | | |
| | | | | | TTT | | | | | TTT | | | |
| G | T | p.Ala1270Ser | PKD1 | HDR | CTGATG | 51 | CATG | 104 | HDR | TGATGC | 155 | ATGT | 206 |
| | | | | | CCCTGC | | | | | CCTGCT | | | |
| | | | | | TGGCAG | | | | | GGCAGC | | | |
| | | | | | CCC | | | | | CCA | | | |
| C | G | p.Leu2073Val | PKD1 | HDR | TCTACC | 52 | TACC | 105 | HDR | ACCTGC | 156 | CGTG | 207 |
| | | | | | TGCAGC | | | | | AGCCCG | | | |
| | | | | | CCGGGG | | | | | GGGACT | | | |
| | | | | | ACT | | | | | ACC | | | |
| C | A | p.Thr1262Asn | SLX4 | | | | | | HDR | CAGCCC | 157 | GGG | 208 |
| | | | | | | | | | | CAGCAG | | | |
| | | | | | | | | | | TAGGGC | | | |
| | | | | | | | | | | CA | | | |

In Table 2, “ABE” refers to Adenine Base Editor; “CBE” refers to Cytosine Base Editor; “HDR” refers to homology directed repair; and “PAM” refers to protospacer adjacent motif

TABLE 3

| | | | | | | SEQ | | SEQ | | SEQ | | SEQ |
|---|---|---|---|---|---|---|---|---|---|---|---|---|
| Gene | Coor- | AA_ | Ele- | Mam- | | ID | | ID | | ID | | ID |
| name | dinates | sub. | phant | moth | SSODN+ | NO: | SSODN− | NO: | gRNA− | NO: | gRNA+ | NO: |
| APOB | scaffold_ | p.Ala | G | A | GCCTGGGAAG | 209 | GTGTTCTGAC | 234 | 22: | 258 | 20: | 282 |
| | 20: | 424 | | | GCCCCCTCAT | | CAAAGGACGG | | CCTCTTTTGG | | CAATCTCTTA | |
| | 32822225- | Val | | | CAGCATGAGA | | TGATAGTACA | | CTACAGATCC | | TCCACTGGAG | |
| | 32822225 | | | | TAGGCAGCCA | | ATAGTCCCCT | | \|7: | | \|6: | |
| | | | | | ATCTCTTATC | | CTTTTGGCTA | | GATCCAGGAA | | CATCGAAGAA | |
| | | | | | CACTGGAGAG | | CAGATCCAGG | | GCCCTTCTTC | | AGCCTGAAGA | |
| | | | | | GCACCATCGA | | AAGCCCTTCT | | \|6: | | \|7: | |
| | | | | | AGAAAACCTG | | TCAGGTTTTC | | CTTCTTCAGG | | ATCGAAGAAA | |
| | | | | | AAGAAGGGCT | | TTCGATGGTG | | CTTTCTTCGA | | GCCTGAAGAA | |
| | | | | | TCCTGGATCT | | CCTCTCCAGT | | | | \|15: | |
| | | | | | GTAGCCAAAA | | GGATAAGAGA | | | | AAGCCTGAAG | |
| | | | | | GAGGGGACTA | | TTGGCTGCCT | | | | AAGGGCTTCC | |
| | | | | | TTGTACTATC | | ATCTCATGCT | | | | | |
| | | | | | ACCGTCCTTT | | GATGAGGGGG | | | | | |
| | | | | | GGTCAGAACA | | CCTTCCCAGG | | | | | |
| | | | | | C | | C | | | | | |
| CD109 | scaffold_ | p.Asn | T | C | GCCCAGGGGA | 210 | TTTGAATTGC | 235 | 0: | 259 | | |
| | 0: | 294 | | | AGAAAGATCC | | TAAAGTGAGA | | TGCAAACTTC | | | |
| | 92113390- | Asp | | | ATGTGTTCAT | | AATAAAATTG | | TCTTTTAATG | | | |
| | 92113390 | | | | AAAGCCCATC | | AACTTTTCAA | | \|15: | | | |
| | | | | | TGAAAAATCC | | TAAAACAGAT | | TAATGAGGAA | | | |
| | | | | | ATTACCTTTT | | AAATGGATCT | | GAGATGAAAA | | | |
| | | | | | TCATCTCTTC | | GCAAACTTCT | | | | | |
| | | | | | CTCATCAAAA | | CTTTTGATGA | | | | | |
| | | | | | GAGAAGTTTG | | GGAAGAGATG | | | | | |
| | | | | | CAGATCCATT | | AAAAAGGTAA | | | | | |
| | | | | | TATCTGTTTT | | TGGATTTTTC | | | | | |
| | | | | | ATTGAAAAGT | | AGATGGGCTT | | | | | |
| | | | | | TCAATTTTAT | | TATGAACACA | | | | | |
| | | | | | TTCTCACTTT | | TGGATCTTTC | | | | | |
| | | | | | AGCAATTCAA | | TTCCCCTGGG | | | | | |
| | | | | | A | | C | | | | | |
| COL27A1 | scaffold_ | p.Gln | T | A | GCACAGGGAA | 211 | CCTTCCTCTC | 236 | 18: | 260 | 14: | 283 |
| | 6: | 1265 | | | GGAGTGGGGC | | ACTCTTTTCC | | GAAGGAAAAC | | TGGGAGGCAG | |
| | 45633049- | Leu | | | AAGGGAGGAG | | CTCCTCTCTC | | CGGGCAAGCA | | GAGTCTACCT | |
| | 45633049 | | | | GAGAAAGGGG | | TTCAGGGTCC | | \|10: | | \|3: | |
| | | | | | ATGGTGGGAG | | TGAAGGAAAA | | ACCGGGCAAG | | AGTCTACCTT | |
| | | | | | GCAGGAGTCT | | CCGGGCAAGC | | CAAGGAGAGA | | GGCTCCAGTC | |
| | | | | | ACCTTGGCTC | | AAGGAGAGAA | | \|9: | | | |
| | | | | | CAGTCAGGCC | | GGGCCTGACT | | CCGGGCAAGC | | | |
| | | | | | CTTCTCTCCT | | GGAGCCAAGG | | AAGGAGAGAA | | | |
| | | | | | TGCTTGCCCG | | TAGACTCCTG | | \|0: | | | |
| | | | | | GTTTTCCTTC | | CCTCCCACCA | | CAAGGAGAGA | | | |
| | | | | | AGGACCCTGA | | TCCCCTTTCT | | AGGGCCAGAC | | | |
| | | | | | AGAGAGAGGA | | CCTCCTCCCT | | \|8: | | | |
| | | | | | GGGAAAAGAG | | TGCCCCACTC | | GAAGGGCCAG | | | |
| | | | | | TGAGAGGAAG | | CTTCCCTGTG | | ACTGGAGCCA | | | |
| | | | | | G | | C | | | | | |
| CRP | scaffold_ | p.Leu | A | T | TTCCTCACCT | 212 | ACAAGCCAGG | 237 | 13: | 261 | 1: | 284 |
| | 33: | 110* | | | TGGGCTTCCT | | AGAATACAGC | | CATTGCATTT | | CAAGTCACAC | |
| | 9027519- | | | | ATTCACCCAG | | TTATCTGTGG | | GTGTGTGACT | | ACAAATGCAA | |
| | 9027519 | | | | AACTCAACAA | | GTGGGACTGA | | \|14: | | \|14: | |
| | | | | | TTCCTGAGAC | | AGTAGTTTTC | | ATTGCATTTG | | TGCAATGGTG | |
| | | | | | CGACTCCCAA | | CAGCATCCTG | | TGTGTGACTT | | CAAATGTATC | |
| | | | | | GTCACACACA | | ATACATTTGC | | | | | |
| | | | | | AATGCTATGG | | ACCATAGCAT | | | | | |
| | | | | | TGCAAATGTA | | TTGTGTGTGA | | | | | |
| | | | | | TCAGGATGCT | | CTTGGGAGTC | | | | | |
| | | | | | GGAAAACTAC | | GGTCTCAGGA | | | | | |
| | | | | | TTCAGTCCCA | | ATTGTTGAGT | | | | | |
| | | | | | CCCACAGATA | | TCTGGGTGAA | | | | | |
| | | | | | AGCTGTATTC | | TAGGAAGCCC | | | | | |
| | | | | | TCCTGGCTTG | | AAGGTGAGGA | | | | | |
| | | | | | T | | A | | | | | |
| CRP | scaffold_ | p.Thr | T | C | TAGACAAGAT | 213 | CCAAGAGGAT | 238 | | 262 | 16: | 285 |
| | 33: | 10Ala | | | CTCAGCTACC | | AACCAAAGTT | | | | TCTCTGAAAA | |
| | 9028091- | | | | ATCTGAAACA | | CTGGCCACAC | | | | AGCAATGGAG | |
| | 9028091 | | | | GCACCTCACC | | AGACAGCAAG | | | | \|10: | |
| | | | | | TGTCTCTGAA | | GAGGGAACAT | | | | AAAAAGCAAT | |
| | | | | | AAAGCAATGG | | GGAGAAGCTG | | | | GGAGAGGCTA | |
| | | | | | AGAGGCTAAG | | TTGCTGTGTT | | | | \|6: | |
| | | | | | GAAGGCCAGG | | TCCTGGCCTT | | | | AGCAATGGAG | |
| | | | | | AAACACAGCA | | CCTTAGCCTC | | | | AGGCTAAGGA | |
| | | | | | ACAGCTTCTC | | TCCATTGCTT | | | | \|1: | |
| | | | | | CATGTTCCCT | | TTTCAGAGAC | | | | TGGAGAGGCT | |
| | | | | | CCTTGCTGTC | | AGGTGAGGTG | | | | AAGGAAGGTC | |
| | | | | | TGTGTGGCCA | | CTGTTTCAGA | | | | | |
| | | | | | GAACTTTGGT | | TGGTAGCTGA | | | | | |
| | | | | | TATCCTCTTG | | GATCTTGTCT | | | | | |
| | | | | | G | | A | | | | | |
| DLK1 | scaffold_ | p.G1y | C | G | ATGTCGCAGA | 214 | TGCCCACTTT | 239 | 6: | 263 | 1: | 286 |
| | 9: | 35 | | | GATGACCCTC | | TCCTTCCCGC | | GACCATTGCG | | CAGATCCCAT | |
| | 75817870- | Ala | | | CCAGCCTTCG | | AGGTGCCACC | | TGCCCTCTCC | | TGACGCAGCC | |
| | 75817870 | | | | TTGCAAACAC | | CTGGCTGGCA | | \|6: | | \|4: | |
| | | | | | ACTGCCCGGG | | GGGTCCCCTG | | CCCTCTCCTG | | CCCATTGACG | |
| | | | | | CTCGAAGCAG | | TGTGACCATT | | GCTGCGTCAA | | CAGCCAGGAG | |
| | | | | | ATCCCATTGA | | GCGTGCCCTC | | \|7: | | \|5: | |
| | | | | | CGCAGGCAGG | | TCCTGCCTGC | | CCTCTCCTGG | | CCATTGACGC | |
| | | | | | AGAGGGCACG | | GTCAATGGGA | | CTGCGTCAAT | | AGCCAGGAGA | |
| | | | | | CAATGGTCAC | | TCTGCTTCGA | | | | \|15: | |
| | | | | | ACAGGGGACC | | GCCCGGGCAG | | | | AGCCAGGAGA | |
| | | | | | CTGCCAGCCA | | TGTGTTTGCA | | | | GGGCACGCAA | |
| | | | | | GGGTGGCACC | | ACGAAGGCTG | | | | | |
| | | | | | TGCGGGAAGG | | GGAGGGTCAT | | | | | |
| | | | | | AAAAGTGGGC | | CTCTGCGACA | | | | | |
| | | | | | A | | T | | | | | |
| FN1 | scaffold_ | p.Asp | T | G | AGTCATCTGA | 215 | GTTTCGATTC | 240 | 20: | 264 | 18: | 287 |
| | 3: | 1685 | | | ATAACTTTAT | | TGAGCATAGA | | TGTGGGCTGC | | CAAACAGAAA | |
| | 11686754- | Glu | | | CAACTTTTTC | | CGCTAACCAC | | AAGCCTTCGA | | TGACCATCGA | |
| | 11686754 | | | | ATGGGTGACT | | ATACTCCACT | | \|7: | | | |
| | | | | | TTGATACTGA | | GTGGGCTGCA | | TTCTGTTTGA | | | |
| | | | | | GTTTGCTTTT | | AGCCTTCGAT | | TCTGCAAAAG | | | |
| | | | | | TACCTCTTTT | | GGTCATTTCT | | | | | |
| | | | | | GCAGAGCAAA | | GTTTGCTCTG | | | | | |
| | | | | | CAGAAATGAC | | CAAAAGAGGT | | | | | |
| | | | | | CATCGAAGGC | | AAAAAGCAAA | | | | | |
| | | | | | TTGCAGCCCA | | CTCAGTATCA | | | | | |
| | | | | | CAGTGGAGTA | | AAGTCACCCA | | | | | |
| | | | | | TGTGGTTAGC | | TGAAAAAGTT | | | | | |
| | | | | | GTCTATGCTC | | GATAAAGTTA | | | | | |
| | | | | | AGAATCGAAA | | TTCAGATGAC | | | | | |
| | | | | | C | | T | | | | | |
| FREM1 | scaffold_ | p.Ile | A | G | TATTTCTTTT | 216 | GGGCAGAGTT | 241 | 15: | 265 | 12: | 288 |
| | 6: | 990Val | | | TTGTAGGTGA | | CCCTGTAGGG | | TCCGAATTTA | | TTTACATCAT | |
| | 84145600- | | | | ATTTATCCAT | | TCCATGATTT | | CTTCCCGAGA | | AAATCCATCT | |
| | 84145600 | | | | GAAAAATTTA | | GAAATTCCAT | | \|5: | | \|13: | |
| | | | | | GCCAAAAGGA | | GACATCCGAA | | TGGATTTATG | | TTACATCATA | |
| | | | | | CTTAAACAGT | | TTTACTTCCC | | ATGTAAAGAA | | AATCCATCTC | |
| | | | | | AAGACCATTC | | GAGATGGATT | | | | | |
| | | | | | TTTACGTCAT | | TATGACGTAA | | | | | |
| | | | | | AAATCCATCT | | AGAATGGTCT | | | | | |
| | | | | | CGGGAAGTAA | | TACTGTTTAA | | | | | |
| | | | | | ATTCGGATGT | | GTCCTTTTGG | | | | | |
| | | | | | CATGGAATTT | | CTAAATTTTT | | | | | |
| | | | | | CAAATCATGG | | CATGGATAAA | | | | | |
| | | | | | ACCCTACAGG | | TTCACCTACA | | | | | |
| | | | | | GAACTCTGCC | | AAAAAGAAAT | | | | | |
| | | | | | C | | A | | | | | |
| GHR | scaffold_ | p.Met | A | G | AGTTACATCA | 217 | GCAAGGCAGT | 242 | 15: | 267 | | |
| | 7: | 534 | | | CCACAGAAAG | | CGCGTTGAGG | | ATATGGATGG | | | |
| | 47751631- | Val | | | CCTTACCACT | | ACGAGGCCCT | | AGGTATAGTC | | | |
| | 47751631 | | | | ACTGCTGTGA | | GTGGAGACTG | | \|14: | | | |
| | | | | | GATCAGAGGC | | TATTATATGG | | TATGGATGGA | | | |
| | | | | | AGCAGAACGA | | ATGGAGGTAT | | GGTATAGTCT | | | |
| | | | | | GCACCCAGCT | | AGTCTGGGAC | | \|9: | | | |
| | | | | | CCGAGGTGCC | | AGGCACCTCG | | ATGGAGGTAT | | | |
| | | | | | TGTCCCAGAC | | GAGCTGGGTG | | AGTCTGGGAC | | | |
| | | | | | TATACCTCCA | | CTCGTTCTGC | | \|1: | | | |
| | | | | | TCCATATAAT | | TGCCTCTGAT | | ATAGTCTGGG | | | |
| | | | | | ACAGTCTCCA | | CTCACAGCAG | | ACAGGCATCT | | | |
| | | | | | CAGGGCCTCG | | TAGTGGTAAG | | \|5: | | | |
| | | | | | TCCTCAACGC | | GCTTTCTGTG | | TGGGACAGGC | | | |
| | | | | | GA | | G | | ATCTCGGAGC | | | |
| | | | | | CTGCCTTGC | | TGATGTAACT | | \|6: | | | |
| LPINI | scaffold_ | p.Val | G | A | ATTTGTGTTT | 218 | TAACCTTTGC | 243 | 4: | 268 | 17: | 289 |
| | 20: | 297 | | | TTTAAAGTCC | | AGCTTGTGGC | | CTTCTGCACT | | ACCTAAAAGT | |
| | 21750119- | Met | | | TTCATGTTCC | | AATTCTCCCC | | GTCCTATCCA | | GATTCAGAAT | |
| | 21750119 | | | | CGACCTTCAA | | ACAGCCAGAG | | | | \|2: | |
| | | | | | CACCTAAAAG | | CATTTCTGGG | | | | AGAATTGGTC | |
| | | | | | TGATTCAGAA | | TTATTCTTCT | | | | AGCAAGTCCG | |
| | | | | | TTGGTCAGCA | | GCACTGTCCT | | | | \|3: | |
| | | | | | AGTCCATGGA | | ATCCATGGAC | | | | TGGTCAGCAA | |
| | | | | | TAGGACAGTG | | TTGCTGACCA | | | | GTCCGTGGAT | |
| | | | | | CAGAAGAATA | | ATTCTGAATC | | | | | |
| | | | | | ACCCAGAAAT | | ACTTTTAGGT | | | | | |
| | | | | | GCTCTGGCTG | | GTTGAAGGTC | | | | | |
| | | | | | TGGGGAGAAT | | GGGAACATGA | | | | | |
| | | | | | TGCCACAAGC | | AGGACTTTAA | | | | | |
| | | | | | TGCAAAGGTT | | AAAACACAAA | | | | | |
| | | | | | A | | T | | | | | |
| MLXIPL | scaffold_ | p.Ala2 | G | C | TAATAAGGCG | 219 | TGTCCGAGTC | 244 | 7: | 269 | 17: | 290 |
| | 45: | Pro | | | CGCAGGCCAC | | GGTGTCCGGG | | GGCCAGACCC | | CGCACCGGGC | |
| | 17547012- | | | | GCGAGCGGCG | | CTCGGGGCGG | | GCCAGCGCCC | | AGGGCGGCCG | |
| | 17547012 | | | | CGGCGGCCGG | | CCCGCGCGCC | | \|5: | | \|11: | |
| | | | | | GCGCACCGGG | | CTGCAAGCCC | | CAGCGCCCCG | | GGGCAGGGCG | |
| | | | | | CAGGGCGGCC | | GCGGCCAGAC | | GCCAAAGCCA | | GCCGTGGCCA | |
| | | | | | GTGGCCATGG | | CCGCCAGCGC | | \|11: | | \|5: | |
| | | | | | CTTTGCCCGG | | CCCGGGCAAA | | CCCGGCCAAA | | GGCGGCCGTG | |
| | | | | | GGCGCTGGCG | | GCCATGGCCA | | GCCATGGCCA | | GCCATGGCTT | |
| | | | | | GGTCTGGCCG | | CGGCCGCCCT | | | | \|1: | |
| | | | | | CGGGCTTGCA | | GCCCGGTGCG | | | | GCCGTGGCCA | |
| | | | | | GGGCGCGCGG | | CCCGGCCGCC | | | | TGGCTTTGGC | |
| | | | | | GCCGCCCCGA | | GCGCCGCTCG | | | | \|0: | |
| | | | | | GCCCGGACAC | | CGTGGCCTGC | | | | CCGTGGCCAT | |
| | | | | | CGACTCGGAC | | GCGCCTTATT | | | | GGCTTTGGCC | |
| | | | | | A | | A | | | | \|1: | |
| | | | | | | | | | | | CGTGGCCATG | |
| | | | | | | | | | | | GCTTTGGCCG | |
| | | | | | | | | | | | \|7: | |
| | | | | | | | | | | | CATGGCTTTG | |
| | | | | | | | | | | | GCCGGGGCGC | |
| | | | | | | | | | | | \|10: | |
| | | | | | | | | | | | GGCTTTGGCC | |
| | | | | | | | | | | | GGGGCGCTGG | |
| | | | | | | | | | | | \|11: | |
| | | | | | | | | | | | GCTTTGGCCG | |
| | | | | | | | | | | | GGGCGCTGGC | |
| | | | | | | | | | | | \|16: | |
| | | | | | | | | | | | GGCCGGGGCG | |
| | | | | | | | | | | | CTGGCGGGTC | |
| PER2 | scaffold_ | p.Asn | A | T | AGGTACCTGG | 220 | GACACACAAC | 245 | 10: | 270 | 11: | 291 |
| | 55: | 614 | | | AGAGCTGCAG | | CTCACACGCT | | TGTCGTCCTG | | TGAGCTCCCA | |
| | 1026156- | Tyr | | | CGAGGCTGCC | | CCACGGCTCA | | CGCTTGTCGC | | GCCGACACTC | |
| | 1026156 | | | | ACACTGAAGA | | AAGCAAACAC | | \|2: | | \|15: | |
| | | | | | GGAAGTATGA | | ACTACCTCCT | | TGCGCTTGTC | | TGAATGCCAG | |
| | | | | | GCTCCCAGCC | | GTCGTCCTGC | | GCTGGCATTC | | CGACAAGCGC | |
| | | | | | GACACTCAGG | | GCTTGTCGCT | | \|1: | | | |
| | | | | | CCCTGTATGC | | GGCATACAGG | | GCGCTTGTCG | | | |
| | | | | | CAGCGACAAG | | GCCTGAGTGT | | CTGGCATTCA | | | |
| | | | | | CGCAGGACGA | | CGGCTGGGAG | | \|11: | | | |
| | | | | | CAGGAGGTAG | | CTCATACTTC | | GGCATTCAGG | | | |
| | | | | | TGTGTTTGCT | | CTCTTCAGTG | | GCCTGAGTGT | | | |
| | | | | | TTGAGCCGTG | | TGGCAGCCTC | | \|15: | | | |
| | | | | | GAGCGTGTGA | | GCTGCAGCTC | | TTCAGGGCCT | | | |
| | | | | | GGTTGTGTGT | | TCCAGGTACC | | GAGTGTCGGC | | | |
| | | | | | C | | T | | \|16: | | | |
| | | | | | | | | | TCAGGGCCTG | | | |
| | | | | | | | | | AGTGTCGGCT | | | |
| PER2 | scaffold_ | p.Tyr | A | G | TTACCAATTT | 221 | ACTCAGGGGG | 246 | 20: | 271 | 0: | 292 |
| | 55: | 1233 | | | CCCGTTTTCT | | GTCCACTTTC | | GCTTTGCTGA | | CAACTTTTGT | |
| | 1038555- | Cys | | | TTTAAGGACT | | TTCCTCTTTG | | GTCCCAGAGC | | GCACCGTATG | |
| | 1038555 | | | | GTGTTTACTG | | GTGTCTGTGG | | \|2: | | \|18: | |
| | | | | | TGAAAACAAG | | CTTTGCTGAG | | GCAGGAATAT | | TGAGGAAGAT | |
| | | | | | GGGAAAGGCA | | TCCCAGAGCA | | CTTCCTCATA | | ATTCCTGCTC | |
| | | | | | ACTTTTGTGC | | GGAATATCTT | | | | | |
| | | | | | ACCGTGTGAG | | CCTCACACGG | | | | | |
| | | | | | GAAGATATTC | | TGCACAAAAG | | | | | |
| | | | | | CTGCTCTGGG | | TTGCCTTTCC | | | | | |
| | | | | | ACTCAGCAAA | | CCTTGTTTTC | | | | | |
| | | | | | GCCACAGACA | | ACAGTAAACA | | | | | |
| | | | | | CCAAAGAGGA | | CAGTCCTTAA | | | | | |
| | | | | | AGAAAGTGGA | | AAGAAAACGG | | | | | |
| | | | | | CCCCCCTGAG | | GAAATTGGTA | | | | | |
| | | | | | T | | A | | | | | |
| PKD1 | scaffold_ | p.Met | T | C | CTGCCTTGCC | 222 | TCCTGAGGGG | 247 | 14: | 272 | 18: | 293 |
| | 53: | 505 | | | TGGACACCTA | | CTGCGAGGGC | | GGTCCCTGCA | | CCCAGGCCCC | |
| | 13911026- | Thr | | | CCTTCACTGC | | CTCCTGCTGC | | GGTCCCCAAT | | GTGTGGGATG | |
| | 13911026 | | | | ACACTCCACC | | ACCAGGGGAC | | \|1: | | \|3: | |
| | | | | | CCCAGGCCCC | | TCAAGGGTCC | | CCCCAATAGG | | GGATGCGGAG | |
| | | | | | GTGTGGGATG | | CTGCAGGTCC | | CGCTCCCATG | | AATGTCCTCA | |
| | | | | | CGGAGAATGT | | CCAATAGGCG | | | | \|2: | |
| | | | | | CCTCACGGGA | | CTCCCGTGAG | | | | GATGCGGAGA | |
| | | | | | GCGCCTATTG | | GACATTCTCC | | | | ATGTCCTCAT | |
| | | | | | GGGACCTGCA | | GCATCCCACA | | | | \|10: | |
| | | | | | GGGACCCTTG | | CGGGGCCTGG | | | | GTCCTCATGG | |
| | | | | | AGTCCCCTGG | | GGGTGGAGTG | | | | GAGCGCCTAT | |
| | | | | | TGCAGCAGGA | | TGCAGTGAAG | | | | \|11: | |
| | | | | | GGCCCTCGCA | | GTAGGTGTCC | | | | TCCTCATGGG | |
| | | | | | GCCCCTCAGG | | AGGCAAGGCA | | | | AGCGCCTATT | |
| | | | | | A | | G | | | | \|12: | |
| | | | | | | | | | | | CCTCATGGGA | |
| | | | | | | | | | | | GCGCCTATTG | |
| PKD1 | scaffold_ | p.Leu | C | G | GTGGTCTTCC | 223 | TCACCGCAGC | 248 | 13: | 273 | 20: | 294 |
| | 53: | 2073 | | | ACTGGGACTT | | CTGTGCCACA | | CACCTGTACA | | GCAGGCAACA | |
| | 13918109- | Val | | | CGGGGATGGG | | AAGAAGCTGA | | CGGTAGTCCC | | GCAGAGCCCT | |
| | 13918109 | | | | GCCCCAGTGC | | CCAGGTTGGA | | \|12: | | \|5: | |
| | | | | | AGGCAACAGC | | CGCGTTCACC | | ACCTGTACAC | | ACCCACATCT | |
| | | | | | AGAGCCCTGG | | TGTACACGGT | | GGTAGTCCCC | | ACCTGCAGCC | |
| | | | | | GCTACCCACA | | AGTCCCCGGG | | \|5: | | \|6: | |
| | | | | | TCTACGTGCA | | CTGCACGTAG | | CACGGTAGTC | | CCCACATCTA | |
| | | | | | GCCCGGGGAC | | ATGTGGGTAG | | CCCGGGCTGC | | CCTGCAGCCC | |
| | | | | | TACCGTGTAC | | CCCAGGGCTC | | \|4: | | \|7: | |
| | | | | | AGGTGAACGC | | TGCTGTTGCC | | CCCCGGGCTG | | CCACATCTAC | |
| | | | | | GTCCAACCTG | | TGCACTGGGG | | CAGGTAGATG | | CTGCAGCCCG | |
| | | | | | GTCAGCTTCT | | CCCCATCCCC | | \|5: | | | |
| | | | | | TTGTGGCACA | | GAAGTCCCAG | | CCCGGGCTGC | | | |
| | | | | | GGCTGCGGTG | | TGGAAGACCA | | AGGTAGATGT | | | |
| | | | | | A | | C | | \|14: | | | |
| | | | | | | | | | CAGGTAGATG | | | |
| | | | | | | | | | TGGGTAGCCC | | | |
| | | | | | | | | | \|15: | | | |
| | | | | | | | | | AGGTAGATGT | | | |
| | | | | | | | | | GGGTAGCCCA | | | |
| SLX4 | scaffold_ | p.Val | G | A | GAGCTTATCC | 224 | CCTCCTCCTC | 249 | 20: | 274 | 7: | 295 |
| | 53: | 92Met | | | TCATGGGTCT | | CAGGCTCTCC | | CCTGCCCCAG | | TCCCCTGCCA | |
| | 11683192- | | | | CCGGTGCTTT | | TCAAGTGTGG | | CTCCTGCTGC | | CAGAGAACGA | |
| | 11683192 | | | | TCCCCAGGCT | | GTACTCGTTC | | \|19: | | \|1: | |
| | | | | | GTGAGCCCGG | | CTGCCCCAGC | | CTGCCCCAGC | | CACAGAGAAC | |
| | | | | | GTCCCCTGCC | | TCCTGCTGCA | | TCCTGCTGCA | | GACGGCGTGA | |
| | | | | | ACAGAGAACG | | GGGCCAAGGC | | \|13: | | \|7: | |
| | | | | | ACGGCATGAT | | CATCATGCCG | | CAGCTCCTGC | | GAACGACGGC | |
| | | | | | GGCCTTGGCC | | TCGTTCTCTG | | TGCAGGGCCA | | GTGATGGCCT | |
| | | | | | CTGCAGCAGG | | TGGCAGGGGA | | \|11: | | | |
| | | | | | AGCTGGGGCA | | CCCGGGCTCA | | CATCACGCCG | | | |
| | | | | | GGAACGAGTA | | CAGCCTGGGG | | TCGTTCTCTG | | | |
| | | | | | CCCACACTTG | | AAAAGCACCG | | \|15: | | | |
| | | | | | AGGAGAGCCT | | GAGACCCATG | | ACGCCGTCGT | | | |
| | | | | | GGAGGAGGAG | | AGGATAAGCT | | TCTCTGTGGC | | | |
| | | | | | G | | C | | \|16: | | | |
| | | | | | | | | | CGCCGTCGTT | | | |
| | | | | | | | | | CTCTGTGGCA | | | |
| SSFA2 | scaffold_ | p.Asn | T | C | CTTCACTTTG | 225 | AAGAAGAAAG | 250 | 9: | 275 | | |
| | 3: | 287Asp | | | TTCCCCTTCA | | ACTCATCTTT | | AGGTAGTTCA | | | |
| | 45240725- | | | | GTTTCTCGGT | | CTTGCTGGCT | | GCAGCTGTTT | | | |
| | 45240725 | | | | TCAAGCTACT | | ACAGTTAAAG | | | | | |
| | | | | | ACTTGCTTCA | | AGGAGGCATC | | | | | |
| | | | | | TCTGAAAGTT | | AGGTAGTTCA | | | | | |
| | | | | | TGTCAATGTC | | GCAGCTGTTT | | | | | |
| | | | | | AACATCCTCC | | TGGAGGATGT | | | | | |
| | | | | | AAAACAGCTG | | TGACATTGAC | | | | | |
| | | | | | CTGAACTACC | | AAACTTTCAG | | | | | |
| | | | | | TGATGCCTCC | | ATGAAGCAAG | | | | | |
| | | | | | TCTTTAACTG | | TAGTAGCTTG | | | | | |
| | | | | | TAGCCAGCAA | | AACCGAGAAA | | | | | |
| | | | | | GAAAGATGAG | | CTGAAGGGGA | | | | | |
| | | | | | TCTTTCTTCT | | ACAAAGTGAA | | | | | |
| | | | | | T | | G | | | | | |
| TCOF1 | scaffold_ | p.Arg | G | A | AGGCCTGGCC | 226 | TCTCCCGACA | 251 | | | 15: | 296 |
| | 1: | 1209 | | | CCTGAGTGAG | | GCTTCCGCTT | | | | GAAGGTCCTG | |
| | 69924513- | Lys | | | GCCCAGGTGC | | GAGGCCTCCT | | | | GCTGAGTTGC | |
| | 69924513 | | | | AGGCCTCAGT | | CGGGCCTTCT | | | | \|4: | |
| | | | | | GGCGAAGGTC | | TGCTGCTCTC | | | | CTGAGTTGCT | |
| | | | | | CTGGCTGAGT | | CTTGGCAGCA | | | | GGAGCAGAAG | |
| | | | | | TGCTGGAGCA | | TCCGCAGCCT | | | | \|3: | |
| | | | | | GAAGAAGAAA | | TTTTCTTCTT | | | | GCTGGAGCAG | |
| | | | | | AAGGCTGCGG | | CTGCTCCAGC | | | | AAGAGGAAAA | |
| | | | | | ATGCTGCCAA | | AACTCAGCCA | | | | \|9: | |
| | | | | | GGAGAGCAGC | | GGACCTTCGC | | | | GCAGAAGAGG | |
| | | | | | AAGAAGGCCC | | CACTGAGGCC | | | | AAAAAGGCTG | |
| | | | | | GAGGAGGCCT | | TGCACCTGGG | | | | | |
| | | | | | CAAGCGGAAG | | CCTCACTCAG | | | | | |
| | | | | | CTGTCGGGAG | | GGGCCAGGCC | | | | | |
| | | | | | A | | T | | | | | |
| TRPM8 | scaffold_ | p.Arg | C | T | CTAACATCTA | 227 | TCGCGAGCCT | 252 | 22: | 276 | 15: | 297 |
| | 55: | 368 | | | CCCAACAGCA | | GGTGGAGATG | | TTCCATCATC | | CTGTTTCCTC | |
| | 5192157- | His | | | ACTCACCGAT | | GAGGACATCT | | AAGGAGAAGT | | CTCCGGAAGC | |
| | 5192157 | | | | TTGATCCAAC | | TGACACCTTC | | \|1: | | \|14: | |
| | | | | | TCTCTGTTTC | | CATCATCAAG | | GGTGCGCTTT | | TGTTTCCTCC | |
| | | | | | CTCCTCCGGA | | GAGAAGTTGG | | CTGCCCCGTA | | TCCGGAAGCC | |
| | | | | | AGCCGGGACA | | TGCGCTTTCT | | \|7: | | \|3: | |
| | | | | | CCGTATGGGG | | GCCCCATACG | | TTCTGCCCCG | | CCGGAAGCCG | |
| | | | | | CAGAAAGCGC | | GTGTCCCGGC | | TACGGTGTCC | | GGACACCGTA | |
| | | | | | ACCAACTTCT | | TTCCGGAGGA | | \|14: | | \|2: | |
| | | | | | CCTTGATGAT | | GGAAACAGAG | | CCGTACGGTG | | CGGAAGCCGG | |
| | | | | | GGAAGGTGTC | | AGTTGGATCA | | TCCCGGCTTC | | GACACCGTAC | |
| | | | | | AAGATGTCCT | | AATCGGTGAG | | | | \|1: | |
| | | | | | CCATCTCCAC | | TTGCTGTTGG | | | | GGAAGCCGGG | |
| | | | | | CAGGCTCGCG | | GTAGATGTTA | | | | ACACCGTACG | |
| | | | | | A | | G | | | | | |
| ADTRP | scaffold_ | p.Val | G | A | CAGTTTGTCT | 228 | AGAGACTTTT | 253 | 12: | 277 | 17: | 298 |
| | 44: | 121 | | | TTTTGTCGTT | | GGATTTCTGT | | ACTGCGTGAT | | CCGAGAACTC | |
| | 18092938- | Ile | | | CTGGGCACTC | | GTTCCCCACT | | TCAGCCATTT | | GTTTACTCAA | |
| | 18092938 | | | | TATCTGTATG | | CTTTCCCATC | | \|4: | | \|9: | |
| | | | | | ACCGAGAACT | | ACTCACCACT | | ATTTTGGAAA | | TAGATAACGT | |
| | | | | | CGTTTACTCA | | GCGTGATTCA | | GACGTTATCT | | CTTTCCAAAA | |
| | | | | | AAGGTCCTAG | | GCCATTTTGG | | | | | |
| | | | | | ATAACATCTT | | AAAGATGTTA | | | | | |
| | | | | | TCCAAAATGG | | TCTAGGACCT | | | | | |
| | | | | | CTGAATCACG | | TTGAGTAAAC | | | | | |
| | | | | | CAGTGGTGAG | | GAGTTCTCGG | | | | | |
| | | | | | TGATGGGAAA | | TCATACAGAT | | | | | |
| | | | | | GAGTGGGGAA | | AGAGTGCCCA | | | | | |
| | | | | | CACAGAAATC | | GAACGACAAA | | | | | |
| | | | | | CAAAAGTCTC | | AAGACAAACT | | | | | |
| | | | | | T | | G | | | | | |
| KRT3 | scaffold_ | p.Tyr | T | G | CTGCAGCTCATT | 230 | TTGTACCTGGGA | 255 | 11: | 279 | 14: | 300 |
| 5 | 31: | 417 | | | CGGATGGTGCCA | | GACCAGGGTAA | | CAACTATTCA | | GAGACAGGG | |
| | 23302945- | Ser | | | GGACTACAGGA | | TCTGAACATTTT | | CCTTCCAAGT | | GAGTCCCGACT\|1 | |
| | 23302945 | | | | GGCCGCCGGGA | | CTTTCTCCTAGG | | \|12: | | 0: | |
| | | | | | GACAGGGGAGT | | CTCCCCTGTAAC | | AACTATTCAC | | CAGGGGAGTC | |
| | | | | | CCCGACTTGGAA | | CCATGTGCCTTC | | CTTCCAAGTC | | CCGACTTGGA\|4: | |
| | | | | | GGTGAAGAGTT | | AACTCTTCACCT | | | | CTTGGAAGGTGA | |
| | | | | | GAAGGCACATG | | TCCAAGTCGGGA | | | | ATAGTTGA\|11: | |
| | | | | | GGTTACAGGGG | | CTCCCCTGTCTC | | | | G | |
| | | | | | AGCCTAGGAGA | | CCGGCGGCCTCC | | | | GTGAATAGTTGA | |
| | | | | | AAGAAAATGTTC | | TGTAGTCCTGGC | | | | AGGCACA\|12: | |
| | | | | | AGATTACCCTGG | | ACCATCCGAATG | | | | GT | |
| | | | | | TCTCCCAGGTAC | | AGCTGCAG | | | | GAATAGTTGAA | |
| | | | | | AA | | | | | | GGCACAT | |
| APOB | scaffold_ | p.Ala | G | A | GCCTGGGAAGG | 209 | GTGTTCTGACCA | 234 | 22: | 301 | 20: | 362 |
| | 20: | 424 | | | CCCCCTCATCAG | | AAGGACGGTGA | | CCTCTTTTGG | | CAATCTCTTA | |
| | 32822225- | Val | | | CATGAGATAGG | | TAGTACAATAGT | | CTACAGATCC | 302 | TCCACTGGAG | 363 |
| | 32822225 | | | | CAGCCAATCTCT | | CCCCTCTTTTGG | | 7: | | 6: | |
| | | | | | TATCCACTGGAG | | CTACAGATCCAG | | GATCCAGGAA | 303 | CATCGAAGAA | 364 |
| | | | | | AGGCACCATCG | | GAAGCCCTTCTT | | GCCCTTCTTC | | AGCCTGAAGA | |
| | | | | | AAGAAAACCTG | | CAGGTTTTCTTC | | 6: | | 7: | 365 |
| | | | | | AAGAAGGGCTT | | GATGGTGCCTCT | | CTTCTTCAGGC | | ATCGAAGAAA | |
| | | | | | CCTGGATCTGTA | | CCAGTGGATAA | | TTTCTTCGA | | GCCTGAAGAA | |
| | | | | | GCCAAAAGAGG | | GAGATTGGCTGC | | | | 15: | |
| | | | | | GGACTATTGTAC | | CTATCTCATGCT | | | | AAGCCTGAAG | |
| | | | | | TATCACCGTCCT | | GATGAGGGGGC | | | | AAGGGCTTCC | |
| | | | | | TTGGTCAGAACA | | CTTCCCAGGC | | | | | |
| | | | | | C | | | | | | | |
| CD109 | scaffold_ | p.Asn | T | C | GCCCAGGGGAA | 210 | TTTGAATTGCTA | 235 | 0: | 304 | | |
| | 0: | 294 | | | GAAAGATCCAT | | AAGTGAGAAAT | | TGCAAACTTCT | | | |
| | 92113390- | Asp | | | GTGTTCATAAAG | | AAAATTGAACTT | | CTTTTAATG | | | |
| | 92113390 | | | | CCCATCTGAAAA | | TTCAATAAAACA | | 15: | 305 | | |
| | | | | | ATCCATTACCTT | | GATAAATGGATC | | TAATGAGGAA | | | |
| | | | | | TTTCATCTCTTC | | TGCAAACTTCTC | | GAGATGAAAA | | | |
| | | | | | CTCATCAAAAGA | | TTTTGATGAGGA | | | | | |
| | | | | | GAAGTTTGCAGA | | AGAGATGAAAA | | | | | |
| | | | | | TCCATTTATCTG | | AGGTAATGGATT | | | | | |
| | | | | | TTTTATTGAAAA | | TTTCAGATGGGC | | | | | |
| | | | | | GTTCAATTTTAT | | TTTATGAACACA | | | | | |
| | | | | | TTCTCACTTTAG | | TGGATCTTTCTT | | | | | |
| | | | | | CAATTCAAA | | CCCCTGGGC | | | | | |
| COL2 | scaffold_ | p.Gln | T | A | GCACAGGGAAG | 211 | CCTTCCTCTCAC | 236 | 18: | 306 | 14: | 366 |
| 7A1 | 6: | 126 | | | GAGTGGGGCAA | | TCTTTTCCCTCC | | GAAGGAAAA | | TGGGAGGCA | |
| | 45633049- | 5Leu | | | GGGAGGAGGAG | | TCTCTCTTCAG | | CCGGGCAAGCA | | GGAGTCTACCT | |
| | 45633049 | | | | AAAGGGGATGG | | GGTCCTGAAGGA | | 10: | 307 | 3: | 367 |
| | | | | | TGGGAGGCAGG | | AAACCGGGCAAG | | ACCGGGCAA | | AGTCTACCTTG | |
| | | | | | AGTCTACCTTGG | | CAAGGAGAGAA | | GCAAGGAGAGA | | GCTCCAGTC | |
| | | | | | CTCCAGTCAGGC | | GGGCCTGACTGG | | 9: | 308 | | |
| | | | | | CCTTCTCTCCTT | | AGCCAAGGTAG | | CCGGGCAAGC | | | |
| | | | | | GCTTGCCCGGTT | | ACTCCTGCCTCC | | AAGGAGAGAA | | | |
| | | | | | TTCCTTCAGGAC | | CACCATCCCCTT | | 0: | 309 | | |
| | | | | | CCTGAAGAGAG | | TCTCCTCCTCCC | | CAAGGAGAGA | | | |
| | | | | | AGGAGGGAAAA | | TTGCCCCACTCC | | AGGGCCAGAC | | | |
| | | | | | GAGTGAGAGGA | | TTCCCTGTGC | | 8: | 310 | | |
| | | | | | AGG | | | | GAAGGGCCAG | | | |
| | | | | | | | | | ACTGGAGCCA | | | |
| CRP | scaffold_ | p.Leu | A | T | TTCCTCACCTTG | 212 | ACAAGCCAGGA | 237 | 13: | 311 | 1: | 368 |
| | 33: | 110 | | | GGCTTCCTATTC | | GAATACAGCTTA | | CATTGCATTT | | CAAGTCACAC | |
| | 9027519- | * | | | ACCCAGAACTCA | | TCTGTGGGTGGG | | GTGTGTGACT | | ACAAATGCAA | |
| | 9027519 | | | | ACAATTCCTGAG | | ACTGAAGTAGTT | | 14: | 312 | 14: | 369 |
| | | | | | ACCGACTCCCAA | | TTCCAGCATCCT | | ATTGCATTTG | | TGCAATGGTG | |
| | | | | | GTCACACACAA | | GATACATTTGCA | | TGTGTGACTT | | CAAATGTATC | |
| | | | | | ATGCTATGGTGC | | CCATAGCATTTG | | | | | |
| | | | | | AAATGTATCAGG | | TGTGTGACTTGG | | | | | |
| | | | | | ATGCTGGAAAA | | GAGTCGGTCTCA | | | | | |
| | | | | | CTACTTCAGTCC | | GGAATTGTTGAG | | | | | |
| | | | | | CACCCACAGATA | | TTCTGGGTGAAT | | | | | |
| | | | | | AGCTGTATTCTC | | AGGAAGCCCAA | | | | | |
| | | | | | CTGGCTTGT | | GGTGAGGAA | | | | | |
| CRP | scaffold_ | p.Thr | T | C | TAGACAAGATCT | 213 | CCAAGAGGATA | 238 | | | 16: | 370 |
| | 33: | 10Ala | | | CAGCTACCATCT | | ACCAAAGTTCTG | | | | TCTCTGAAAA | |
| | 9028091- | | | | GAAACAGCACC | | GCCACACAGAC | | | | AGCAATGGAG | |
| | 9028091 | | | | TCACCTGTCTCT | | AGCAAGGAGGG | | | | 10: | 371 |
| | | | | | GAAAAAGCAAT | | AACATGGAGAA | | | | AAAAAGCAA | |
| | | | | | GGAGAGGCTAA | | GCTGTTGCTGTG | | | | TGGAGAGGCTA | |
| | | | | | GGAAGGCCAGG | | TTTCCTGGCCTT | | | | 6: | 372 |
| | | | | | AAACACAGCAA | | CCTTAGCCTCTC | | | | AGCAATGGAG | |
| | | | | | CAGCTTCTCCAT | | CATTGCTTTTTC | | | | AGGCTAAGGA | |
| | | | | | GTTCCCTCCTTG | | AGAGACAGGTG | | | | 1: | 373 |
| | | | | | CTGTCTGTGTGG | | AGGTGCTGTTTC | | | | TGGAGAGGCT | |
| | | | | | CCAGAACTTTGG | | AGATGGTAGCTG | | | | AAGGAAGGTC | |
| | | | | | TTATCCTCTTGG | | AGATCTTGTCTA | | | | | |
| DLK1 | scaffold_ | p.Gly | C | G | ATGTCGCAGAG | 214 | TGCCCACTTTTC | 239 | 6: | 313 | 1: | 374 |
| | 9: | 35 | | | ATGACCCTCCCA | | CTTCCCGCAGGT | | GACCATTGCG | | CAGATCCCATT | |
| | 75817870- | Ala | | | GCCTTCGTTGCA | | GCCACCCTGGCT | | TGCCCTCTCC | | GACGCAGCC | |
| | 75817870 | | | | AACACACTGCCC | | GGCAGGGTCCCC | | 6: | 314 | 4: | 375 |
| | | | | | GGGCTCGAAGC | | TGTGTGACCATT | | CCCTCTCCTG | | CCCATTGACGC | |
| | | | | | AGATCCCATTGA | | GCGTGCCCTCTC | | GCTGCGTCAA | | AGCCAGGAG | |
| | | | | | CGCAGGCAGGA | | CTGCCTGCGTCA | | 7: | 315 | 5: | 376 |
| | | | | | GAGGGCACGCA | | ATGGGATCTGCT | | CCTCTCCTGG | | CCATTGACGCA | |
| | | | | | ATGGTCACACAG | | TCGAGCCCGGGC | | CTGCGTCAAT | | GCCAGGAGA | |
| | | | | | GGGACCCTGCCA | | AGTGTGTTTGCA | | | | 15: | 377 |
| | | | | | GCCAGGGTGGC | | ACGAAGGCTGG | | | | AGCCAGGAG | |
| | | | | | ACCTGCGGGAA | | GAGGGTCATCTC | | | | AGGGCACGCAA | |
| | | | | | GGAAAAGTGGG | | TGCGACAT | | | | | |
| | | | | | CA | | | | | | | |
| FN1 | scaffold_ | p.Asp | T | G | AGTCATCTGAAT | 215 | GTTTCGATTCTG | 240 | 20: | 316 | 18: | 378 |
| | 3: | 168 | | | AACTTTATCAAC | | AGCATAGACGCT | | TGTGGGCTGC | | CAAACAGAA | |
| | 11686754- | 5Glu | | | TTTTTCATGGGT | | AACCACATACTC | | AAGCCTTCGA | | ATGACCATCGA | |
| | 11686754 | | | | GACTTTGATACT | | CACTGTGGGCTG | | 7: | 317 | | |
| | | | | | GAGTTTGCTTTT | | CAAGCCTTCGAT | | TTCTGTTTGAT | | | |
| | | | | | TACCTCTTTTGC | | GGTCATTTCTGT | | CTGCAAAAG | | | |
| | | | | | AGAGCAAACAG | | TTGCTCTGCAAA | | | | | |
| | | | | | AAATGACCATCG | | AGAGGTAAAAA | | | | | |
| | | | | | AAGGCTTGCAGC | | GCAAACTCAGTA | | | | | |
| | | | | | CCACAGTGGAGT | | TCAAAGTCACCC | | | | | |
| | | | | | ATGTGGTTAGCG | | ATGAAAAAGTT | | | | | |
| | | | | | TCTATGCTCAGA | | GATAAAGTTATT | | | | | |
| | | | | | ATCGAAAC | | CAGATGACT | | | | | |
| FREM | scaffold_ | p.Ile | A | G | TATTTCTTTTTT | 216 | GGGCAGAGTTCC | 241 | 15: | 318 | 12: | 379 |
| 1 | 6: | 990 | | | GTAGGTGAATTT | | CTGTAGGGTCCA | | TCCGAATTTA | | TTTACATCAT | |
| | 84145600- | Val | | | ATCCATGAAAAA | | TGATTTGAAATT | | CTTCCCGAGA | | AAATCCATCT | |
| | 84145600 | | | | TTTAGCCAAAAG | | CCATGACATCCG | | 5: | 319 | 13: | 380 |
| | | | | | GACTTAAACAGT | | AATTTACTTCCC | | TGGATTTATGA | | TTACATCATA | |
| | | | | | AAGACCATTCTT | | GAGATGGATTTA | | TGTAAAGAA | | AATCCATCTC | |
| | | | | | TACGTCATAAAT | | TGACGTAAAGA | | | | | |
| | | | | | CCATCTCGGGAA | | ATGGTCTTACTG | | | | | |
| | | | | | GTAAATTCGGAT | | TTTAAGTCCTTT | | | | | |
| | | | | | GTCATGGAATTT | | TGGCTAAATTTT | | | | | |
| | | | | | CAAATCATGGAC | | TCATGGATAAAT | | | | | |
| | | | | | CCTACAGGGAA | | TCACCTACAAAA | | | | | |
| | | | | | CTCTGCCC | | AAGAAATA | | | | | |
| GHR | scaffold_ | p.Met | A | G | AGTTACATCACC | 217 | GCAAGGCAGTC | 242 | 15: | 320 | | |
| | 7: | 534 | | | ACAGAAAGCCTT | | GCGTTGAGGAC | | ATATGGATGG | | | |
| | 47751631- | Val | | | ACCACTACTGCT | | GAGGCCCTGTGG | | AGGTATAGTC | | | |
| | 47751631 | | | | GTGAGATCAGA | | AGACTGTATTAT | | 14: | 321 | | |
| | | | | | GGCAGCAGAAC | | ATGGATGGAGG | | TATGGATGGA | | | |
| | | | | | GAGCACCCAGCT | | TATAGTCTGGGA | | 1: | 323 | | |
| | | | | | CCGAGGTGCCTG | | CAGGCACCTCGG | | ATAGTCTGGG | | | |
| | | | | | TCCCAGACTATA | | AGCTGGGTGCTC | | ACAGGCATCT | | | |
| | | | | | CCTCCATCCATA | | GTTCTGCTGCCT | | 5: | 324 | | |
| | | | | | TAATACAGTCTC | | CTGATCTCACAG | | TGGGACAGGC | | | |
| | | | | | CACAGGGCCTCG | | CAGTAGTGGTAA | | ATCTCGGAGC | | | |
| | | | | | TCCTCAACGCGA | | GGCTTTCTGTGG | | 6: | 325 | | |
| | | | | | CTGCCTTGC | | TGATGTAACT | | GGGACAGGCA | | | |
| | | | | | | | | | TCTCGGAGCT | | | |
| LPIN1 | scaffold_ | p.Val | G | A | ATTTGTGTTTTT | 218 | TAACCTTTGCAG | 243 | 4: | 326 | 17: | 381 |
| | 20: | 297 | | | TAAAGTCCTTCA | | CTTGTGGCAATT | | CTTCTGCACTG | | ACCTAAAAGT | |
| | 21750119- | Met | | | TGTTCCCGACCT | | CTCCCCACAGCC | | TCCTATCCA | | GATTCAGAAT | |
| | 21750119 | | | | TCAACACCTAAA | | AGAGCATTTCTG | | | | 2: | 382 |
| | | | | | AGTGATTCAGAA | | GGTTATTCTTCT | | | | AGAATTGGTC | |
| | | | | | TTGGTCAGCAAG | | GCACTGTCCTAT | | | | AGCAAGTCCG | |
| | | | | | TCCATGGATAGG | | CCATGGACTTGC | | | | 3: | 383 |
| | | | | | ACAGTGCAGAA | | TGACCAATTCTG | | | | TGGTCAGCAA | |
| | | | | | GAATAACCCAG | | AATCACTTTTAG | | | | GTCCGTGGAT | |
| | | | | | AAATGCTCTGGC | | GTGTTGAAGGTC | | | | | |
| | | | | | TGTGGGGAGAA | | GGGAACATGAA | | | | | |
| | | | | | TTGCCACAAGCT | | GGACTTTAAAAA | | | | | |
| | | | | | GCAAAGGTTA | | ACACAAAT | | | | | |
| MLXI | scaffold_ | p.Ala | G | C | TAATAAGGCGC | 219 | TGTCCGAGTCGG | 244 | 7: | 327 | 17: | 384 |
| PL | 45: | 2Pro | | | GCAGGCCACGC | | TGTCCGGGCTCG | | GGCCAGACCC | | CGCACCGGGC | |
| | 17547012- | | | | GAGCGGCGCGG | | GGGCGGCCCGC | | GCCAGCGCCC | | AGGGCGGCCG | |
| | 17547012 | | | | CGGCCGGGCGC | | GCGCCCTGCAAG | | 5: | 328 | 11: | 385 |
| | | | | | ACCGGGCAGGG | | CCCGCGGCCAG | | CAGCGCCCCG | | GGGCAGGGC | |
| | | | | | CGGCCGTGGCCA | | ACCCGCCAGCGC | | GCCAAAGCCA | | GGCCGTGGCCA | |
| | | | | | TGGCTTTGCCCG | | CCCGGGCAAAG | | 11: | 329 | 5: | 386 |
| | | | | | GGGCGCTGGCG | | CCATGGCCACGG | | CCCGGCCAAA | | GGCGGCCGTG | |
| | | | | | GGTCTGGCCGCG | | CCGCCCTGCCCG | | GCCATGGCCA | | GCCATGGCTT | |
| | | | | | GGCTTGCAGGGC | | GTGCGCCCGGCC | | | | 1: | 387 |
| | | | | | GCGCGGGCCGC | | GCCGCGCCGCTC | | | | GCCGTGGCCAT | |
| | | | | | CCCGAGCCCGG | | GCGTGGCCTGCG | | | | GGCTTTGGC | |
| | | | | | ACACCGACTCGG | | CGCCTTATTA | | | | 0: | 388 |
| | | | | | ACA | | | | | | CCGTGGCCATG | |
| | | | | | | | | | | | GCTTTGGCC | |
| | | | | | | | | | | | 1: | 389 |
| | | | | | | | | | | | CGTGGCCATG | |
| | | | | | | | | | | | GCTTTGGCCG | |
| | | | | | | | | | | | 7: | 390 |
| | | | | | | | | | | | CATGGCTTTGG | |
| | | | | | | | | | | | CCGGGGCGC | |
| | | | | | | | | | | | 10: | 391 |
| | | | | | | | | | | | GGCTTTGGCC | |
| | | | | | | | | | | | GGGGCGCTGG | |
| | | | | | | | | | | | 11: | 392 |
| | | | | | | | | | | | GCTTTGGCCG | |
| | | | | | | | | | | | GGGCGCTGGC | |
| | | | | | | | | | | | 16: | 393 |
| | | | | | | | | | | | GGCCGGGGC | |
| | | | | | | | | | | | GCTGGCGGGTC | |
| PER2 | scaffold_ | p.Asn | A | T | AGGTACCTGGA | 220 | GACACACAACCT | 245 | 10: | 330 | 11: | 394 |
| | 55: | 614 | | | GAGCTGCAGCG | | CACACGCTCCAC | | TGTCGTCCTG | | TGAGCTCCCA | |
| | 1026156- | Tyr | | | AGGCTGCCACAC | | GGCTCAAAGCA | | CGCTTGTCGC | | GCCGACACTC | |
| | 1026156 | | | | TGAAGAGGAAG | | AACACACTACCT | | 2: | 331 | 15: | 395 |
| | | | | | TATGAGCTCCCA | | CCTGTCGTCCTG | | TGCGCTTGTCG | | TGAATGCCAG | |
| | | | | | GCCGACACTCAG | | CGCTTGTCGCTG | | CTGGCATTC | | CGACAAGCGC | |
| | | | | | GCCCTGTATGCC | | GCATACAGGGC | | 1: | 332 | | |
| | | | | | AGCGACAAGCG | | CTGAGTGTCGGC | | GCGCTTGTCGC | | | |
| | | | | | CAGGACGACAG | | TGGGAGCTCATA | | TGGCATTCA | | | |
| | | | | | GAGGTAGTGTGT | | CTTCCTCTTCAG | | 11: | 333 | | |
| | | | | | TTGCTTTGAGCC | | TGTGGCAGCCTC | | GGCATTCAGG | | | |
| | | | | | GTGGAGCGTGTG | | GCTGCAGCTCTC | | GCCTGAGTGT | | | |
| | | | | | AGGTTGTGTGTC | | CAGGTACCT | | 15: | 334 | | |
| | | | | | | | | | TTCAGGGCCT | | | |
| | | | | | | | | | GAGTGTCGGC | | | |
| | | | | | | | | | 16: | 335 | | |
| | | | | | | | | | TCAGGGCCTG | | | |
| | | | | | | | | | AGTGTCGGCT | | | |
| PER2 | scaffold_ | p.Tyr | A | G | TTACCAATTTCC | 221 | ACTCAGGGGGG | 246 | 20: | 336 | 0: | 396 |
| | 55: | 1233 | | | CGTTTTCTTTTA | | TCCACTTTCTTC | | GCTTTGCTGA | | CAACTTTTGTG | |
| | 1038555- | Cys | | | AGGACTGTGTTT | | CTCTTTGGTGTC | | GTCCCAGAGC | | CACCGTATG | |
| | 1038555 | | | | ACTGTGAAAAC | | TGTGGCTTTGCT | | 2: | 337 | 18: | 397 |
| | | | | | AAGGGGAAAGG | | GAGTCCCAGAG | | GCAGGAATAT | | TGAGGAAGAT | |
| | | | | | CAACTTTTGTGC | | CAGGAATATCTT | | CTTCCTCATA | | ATTCCTGCTC | |
| | | | | | ACCGTGTGAGG | | CCTCACACGGTG | | | | | |
| | | | | | AAGATATTCCTG | | CACAAAAGTTGC | | | | | |
| | | | | | CTCTGGGACTCA | | CTTTCCCCTTGT | | | | | |
| | | | | | GCAAAGCCACA | | TTTCACAGTAAA | | | | | |
| | | | | | GACACCAAAGA | | CACAGTCCTTAA | | | | | |
| | | | | | GGAAGAAAGTG | | AAGAAAACGGG | | | | | |
| | | | | | GACCCCCCTGAG | | AAATTGGTAA | | | | | |
| | | | | | T | | | | | | | |
| PKD1 | scaffold_ | p.Met | T | C | CTGCCTTGCCTG | 222 | TCCTGAGGGGCT | 247 | 14: | 338 | 18: | 398 |
| | 53: | 505 | | | GACACCTACCTT | | GCGAGGGCCTCC | | GGTCCCTGCA | | CCCAGGCCCC | |
| | 13911026- | Thr | | | CACTGCACACTC | | TGCTGCACCAGG | | GGTCCCCAAT | | GTGTGGGATG | |
| | 13911026 | | | | CACCCCCAGGCC | | GGACTCAAGGG | | 1: | 339 | 3: | 399 |
| | | | | | CCGTGTGGGATG | | TCCCTGCAGGTC | | CCCCAATAGG | | GGATGCGGAG | |
| | | | | | CGGAGAATGTCC | | CCCAATAGGCGC | | CGCTCCCATG | | AATGTCCTCA | |
| | | | | | TCACGGGAGCG | | TCCCGTGAGGAC | | | | 2: | 400 |
| | | | | | CCTATTGGGGAC | | ATTCTCCGCATC | | | | GATGCGGAGA | |
| | | | | | CTGCAGGGACCC | | CCACACGGGGC | | | | ATGTCCTCAT | |
| | | | | | TTGAGTCCCCTG | | CTGGGGGTGGA | | | | 10: | 401 |
| | | | | | GTGCAGCAGGA | | GTGTGCAGTGAA | | | | GTCCTCATGG | |
| | | | | | GGCCCTCGCAGC | | GGTAGGTGTCCA | | | | GAGCGCCTAT | |
| | | | | | CCCTCAGGA | | GGCAAGGCAG | | | | 11: | 402 |
| | | | | | | | | | | | TCCTCATGGG | |
| | | | | | | | | | | | AGCGCCTATT | |
| | | | | | | | | | | | 12: | 403 |
| | | | | | | | | | | | CCTCATGGGA | |
| | | | | | | | | | | | GCGCCTATTG | |
| PKD1 | scaffold_ | p.Leu | C | G | GTGGTCTTCCAC | 223 | TCACCGCAGCCT | 248 | 13: | 340 | 20: | 404 |
| | 53: | 2073 | | | TGGGACTTCGGG | | GTGCCACAAAG | | CACCTGTACA | | GCAGGCAAC | |
| | 13918109- | Val | | | GATGGGGCCCC | | AAGCTGACCAG | | CGGTAGTCCC | | AGCAGAGCCCT | |
| | 13918109 | | | | AGTGCAGGCAA | | GTTGGACGCGTT | | 12: | 341 | 5: | 405 |
| | | | | | CAGCAGAGCCCT | | CACCTGTACACG | | ACCTGTACAC | | ACCCACATCTA | |
| | | | | | GGGCTACCCACA | | GTAGTCCCCGGG | | GGTAGTCCCC | | CCTGCAGCC | |
| | | | | | TCTACGTGCAGC | | CTGCACGTAGAT | | 5: | 342 | 6: | 406 |
| | | | | | CCGGGGACTACC | | GTGGGTAGCCCA | | CACGGTAGTCC | | CCCACATCTAC | |
| | | | | | GTGTACAGGTGA | | GGGCTCTGCTGT | | CCGGGCTGC | | CTGCAGCCC | |
| | | | | | ACGCGTCCAACC | | TGCCTGCACTGG | | 4: | 343 | 7: | 407 |
| | | | | | TGGTCAGCTTCT | | GGCCCCATCCCC | | CCCCGGGCTGC | | CCACATCTACC | |
| | | | | | TTGTGGCACAGG | | GAAGTCCCAGTG | | AGGTAGATG | | TGCAGCCCG | |
| | | | | | CTGCGGTGA | | GAAGACCAC | | 5: | 344 | | |
| | | | | | | | | | CCCGGGCTGC | | | |
| | | | | | | | | | AGGTAGATGT | | | |
| | | | | | | | | | 14: | 345 | | |
| | | | | | | | | | CAGGTAGATG | | | |
| | | | | | | | | | TGGGTAGCCC | | | |
| | | | | | | | | | 15: | 346 | | |
| | | | | | | | | | AGGTAGATGT | | | |
| | | | | | | | | | GGGTAGCCCA | | | |
| SLX4 | scaffold_ | p.Val | G | A | GAGCTTATCCTC | 224 | CCTCCTCCTCCA | 249 | 20: | 347 | 7: | 408 |
| | 53: | 92 | | | ATGGGTCTCCGG | | GGCTCTCCTCAA | | CCTGCCCCAG | | TCCCCTGCCAC | |
| | 11683192- | Met | | | TGCTTTTCCCCA | | GTGTGGGTACTC | | CTCCTGCTGC | | AGAGAACGA | |
| | 11683192 | | | | GGCTGTGAGCCC | | GTTCCTGCCCCA | | 19: | 348 | 1: | 409 |
| | | | | | GGGTCCCCTGCC | | GCTCCTGCTGCA | | CTGCCCCAGC | | CACAGAGAAC | |
| | | | | | ACAGAGAACGA | | GGGCCAAGGCC | | TCCTGCTGCA | | GACGGCGTGA | |
| | | | | | CGGCATGATGGC | | ATCATGCCGTCG | | 13: | 349 | 7: | 410 |
| | | | | | CTTGGCCCTGCA | | TTCTCTGTGGCA | | CAGCTCCTGC | | GAACGACGGC | |
| | | | | | GCAGGAGCTGG | | GGGGACCCGGG | | TGCAGGGCCA | | GTGATGGCCT | |
| | | | | | GGCAGGAACGA | | CTCACAGCCTGG | | 11: | 350 | | |
| | | | | | GTACCCACACTT | | GGAAAAGCACC | | CATCACGCCG | | | |
| | | | | | GAGGAGAGCCT | | GGAGACCCATG | | TCGTTCTCTG | | | |
| | | | | | GGAGGAGGAGG | | AGGATAAGCTC | | 15: | 351 | | |
| | | | | | | | | | ACGCCGTCGT | | | |
| | | | | | | | | | TCTCTGTGGC | | | |
| | | | | | | | | | 16: | 352 | | |
| | | | | | | | | | CGCCGTCGTT | | | |
| | | | | | | | | | CTCTGTGGCA | | | |
| SSFA2 | scaffold_ | p.Asn | T | C | CTTCACTTTGTT | 225 | AAGAAGAAAGA | 250 | 9: | 353 | | |
| | 3: | 287 | | | CCCCTTCAGTTT | | CTCATCTTTCTT | | AGGTAGTTCA | | | |
| | 45240725- | Asp | | | CTCGGTTCAAGC | | GCTGGCTACAGT | | GCAGCTGTTT | | | |
| | 45240725 | | | | TACTACTTGCTT | | TAAAGAGGAGG | | | | | |
| | | | | | CATCTGAAAGTT | | CATCAGGTAGTT | | | | | |
| | | | | | TGTCAATGTCAA | | CAGCAGCTGTTT | | | | | |
| | | | | | CATCCTCCAAAA | | TGGAGGATGTTG | | | | | |
| | | | | | CAGCTGCTGAAC | | ACATTGACAAAC | | | | | |
| | | | | | TACCTGATGCCT | | TTTCAGATGAAG | | | | | |
| | | | | | CCTCTTTAACTG | | CAAGTAGTAGCT | | | | | |
| | | | | | TAGCCAGCAAG | | TGAACCGAGAA | | | | | |
| | | | | | AAAGATGAGTCT | | ACTGAAGGGGA | | | | | |
| | | | | | TTCTTCTT | | ACAAAGTGAAG | | | | | |
| TCOF | scaffold_ | p.Arg | G | A | AGGCCTGGCCCC | 226 | TCTCCCGACAGC | 251 | | | 15: | 411 |
| 1 | 1: | 120 | | | TGAGTGAGGCCC | | TTCCGCTTGAGG | | | | GAAGGTCCTG | |
| | 69924513- | 9Lys | | | AGGTGCAGGCCT | | CCTCCTCGGGCC | | | | GCTGAGTTGC | |
| | 69924513 | | | | CAGTGGCGAAG | | TTCTTGCTGCTC | | | | | |
| | | | | | GTCCTGGCTGAG | | TCCTTGGCAGCA | | | | 4: | 412 |
| | | | | | TTGCTGGAGCAG | | TCCGCAGCCTTT | | | | CTGAGTTGCTG | |
| | | | | | AAGAAGAAAAA | | TTCTTCTTCTGCT | | | | GAGCAGAAG | |
| | | | | | GGCTGCGGATGC | | CCAGCAACTCAG | | | | 3: | 413 |
| | | | | | TGCCAAGGAGA | | CCAGGACCTTCG | | | | GCTGGAGCAG | |
| | | | | | GCAGCAAGAAG | | CCACTGAGGCCT | | | | AAGAGGAAAA | |
| | | | | | GCCCGAGGAGG | | GCACCTGGGCCT | | | | 9: | 414 |
| | | | | | CCTCAAGCGGA | | CACTCAGGGGCC | | | | GCAGAAGAGG | |
| | | | | | AGCTGTCGGGA | | AGGCCT | | | | AAAAAGGCTG | |
| | | | | | GA | | | | | | | |
| TRPM | scaffold_ | p.Arg | C | T | CTAACATCTACC | 227 | TCGCGAGCCTGG | 252 | 22: | 354 | 15: | 415 |
| 8 | 55: | 368 | | | CAACAGCAACTC | | TGGAGATGGAG | | TTCCATCATC | | CTGTTTCCTC | |
| | 5192157- | His | | | ACCGATTTGATC | | GACATCTTGACA | | AAGGAGAAGT | | CTCCGGAAGC | |
| | 5192157 | | | | CAACTCTCTGTT | | CCTTCCATCATC | | 1: | 355 | 14: | 416 |
| | | | | | TCCTCCTCCGGA | | AAGGAGAAGTT | | GGTGCGCTTTC | | TGTTTCCTCC | |
| | | | | | AGCCGGGACAC | | GGTGCGCTTTCT | | TGCCCCGTA | | TCCGGAAGCC | |
| | | | | | CGTATGGGGCA | | GCCCCATACGGT | | 7: | 356 | 3: | 417 |
| | | | | | GAAAGCGCACC | | GTCCCGGCTTCC | | TTCTGCCCCGT | | CCGGAAGCCG | |
| | | | | | AACTTCTCCTTG | | GGAGGAGGAAA | | ACGGTGTCC | | GGACACCGTA | |
| | | | | | ATGATGGAAGG | | CAGAGAGTTGG | | 14: | 357 | 2: | 418 |
| | | | | | TGTCAAGATGTC | | ATCAAATCGGTG | | CCGTACGGTG | | CGGAAGCCGG | |
| | | | | | CTCCATCTCCAC | | AGTTGCTGTTGG | | TCCCGGCTTC | | GACACCGTAC | |
| | | | | | CAGGCTCGCGA | | GTAGATGTTAG | | | | 1: | 419 |
| | | | | | | | | | | | GGAAGCCGGG | |
| | | | | | | | | | | | ACACCGTACG | |
| ADTR | scaffold_ | p.Val | G | A | CAGTTTGTCTTT | 228 | AGAGACTTTTGG | 253 | 12: | 358 | 17: | 420 |
| P | 44: | 121 | | | TTGTCGTTCTGG | | ATTTCTGTGTTC | | ACTGCGTGAT | | CCGAGAACTC | |
| | 18092938- | Ile | | | GCACTCTATCTG | | CCCACTCTTTCC | | TCAGCCATTT | | GTTTACTCAA | |
| | 18092938 | | | | TATGACCGAGA | | CATCACTCACCA | | 4: | 359 | 9: | 421 |
| | | | | | ACTCGTTTACTC | | CTGCGTGATTCA | | ATTTTGGAAAG | | TAGATAACGTC | |
| | | | | | AAAGGTCCTAG | | GCCATTTTGGAA | | ACGTTATCT | | TTTCCAAAA | |
| | | | | | ATAACATCTTTC | | AGATGTTATCTA | | | | | |
| | | | | | CAAAATGGCTG | | GGACCTTTGAGT | | | | | |
| | | | | | AATCACGCAGTG | | AAACGAGTTCTC | | | | | |
| | | | | | GTGAGTGATGG | | GGTCATACAGAT | | | | | |
| | | | | | GAAAGAGTGGG | | AGAGTGCCCAG | | | | | |
| | | | | | GAACACAGAAA | | AACGACAAAAA | | | | | |
| | | | | | TCCAAAAGTCTC | | GACAAACTG | | | | | |
| | | | | | T | | | | | | | |
| KRT3 | scaffold_ | p.Tyr | T | G | CTGCAGCTCATT | 230 | TTGTACCTGGGA | 255 | 11: | 360 | 14: | 422 |
| 5 | 31: | 417 | | | CGGATGGTGCCA | | GACCAGGGTAA | | CAACTATTCA | | GAGACAGGG | |
| | 23302945- | Ser | | | GGACTACAGGA | | TCTGAACATTTT | | CCTTCCAAGT | | GAGTCCCGACT | |
| | 23302945 | | | | GGCCGCCGGGA | | CTTTCTCCTAGG | | 12: | 361 | 10: | 423 |
| | | | | | GACAGGGGAGT | | CTCCCCTGTAAC | | AACTATTCAC | | CAGGGGAGTC | |
| | | | | | CCCGACTTGGAA | | CCATGTGCCTTC | | CTTCCAAGTC | | CCGACTTGGA | |
| | | | | | GGTGAAGAGTT | | ??????TCACCT | | | | 4: | 424 |
| | | | | | GAAGGCACATG | | TCCAAGTCGGGA | | | | CTTGGAAGGT | |
| | | | | | GGTTACAGGGG | | CTCCCCTGTCTC | | | | GAATAGTTGA | |
| | | | | | AGCCTAGGAGA | | CCGGCGGCCTCC | | | | 11: | 425 |
| | | | | | AAGAAAATGTTC | | TGTAGTCCTGGC | | | | GGTGAATAGT | |
| | | | | | AGATTACCCTGG | | ACCATCCGAATG | | | | TGAAGGCACA | |
| | | | | | TCTCCCAGGTAC | | AGCTGCAG | | | | 12: | 426 |
| | | | | | AA | | | | | | GTGAATAGTT | |
| | | | | | | | | | | | GAAGGCACAT | |

1. A viable cell comprising at least one exogenous nucleic acid sequence selected from the group consisting of: the woolly mammoth genes in TABLE 1.

2. The cell of claim 1, wherein the cell expresses a polypeptide encoded by the at least one nucleic acid sequence.

3. The cell of claim 1, wherein the cell is selected from the group consisting of a stem cell, a reprogrammed cell, a fibroblast cell, a mesenchymal cell, a nerve cell, cartilage cell, bone cell, muscle cell, bone cell, fat cell, and epidermal cell.

4. The cell of claim 1, wherein the cell expresses at least one stem cell marker.

5. The cell of claim 4, wherein the stem cell marker is selected from NANOG, SSEA1, SSEA4, or TRA-1-60.

6. The cell of claim 3, wherein the stem cell is an induced stem cell, embryonic stem (ES) cell, or mesenchymal stem cell (MSC).

7.-9. (canceled)

10. The cell of claim 1, wherein the cell is at least one of a cell previously differentiated in vitro into a cell selected from the group consisting of a nerve cell, cartilage cell, bone cell, muscle cell, bone cell, fat cell, or epidermal cell;
does not express an endogenous homologue of the at least one exogenous nucleic acid sequence;
is edited to inhibit expression of an endogenous homologue of the at least one exogenous nucleic acid sequence;
an elephant cell; and
a hyrax cell or manatee cell.

11.-14. (canceled)

15. The cell of claim 10, wherein the elephant cell is an African elephant (Loxodanta Africanus) cell or an Asian elephant (Elephas maximus) cell, wherein the hyrax cell is selected from the group consisting of: Dendrohyrax arboreus cell, a Dendrohyrax dorsalis cell, a Heterohyrax brucei cell, and a Procavia capensis cell, or wherein the manatee cell is selected from the group consisting of: a Trichechus inunguis cell, a Trichechus manatus cell, a Trichechus manatus latirostris cell, a Trichechus manatus manatus cell, and a Trichechus senegalensis cell.

16.-20. (canceled)

21. The cell of claim 1, wherein the cells exhibit one or more phenotypes selected from the group consisting of: a modulation of calcium signals; a modulation of electrophysiological function; a modulation in the rate of protein synthesis, a modulation in metabolic function; and a modulation in the lipid content of the cell membrane as compared to an appropriate control.

22.-23. (canceled)

24. The cell of claim 1, wherein the cell is an elephant cell edited to alter an elephant homologue of the at least one gene.

25. The cell of claim 24, wherein the elephant cell is edited to delete or inhibit the function of at least one gene.

26.-30. (canceled)

31. A non-human organism comprising the cell of claim 1.

32.-43. (canceled)

44. An elephant cell comprising at least one guide RNA listed in TABLES 2 or 3.

45. The elephant cell of claim 44, further expressing an RNA-guided endonuclease guided by the at least one guide RNA.

46.-47. (canceled)

48. A guide RNA comprising a sequence selected from SEQ ID NO: 1 to SEQ ID NO: 426.

49. A nucleic acid encoding a guide RNA of claim 48.

50. The nucleic acid of claim 49, wherein the nucleic acid encoding the guide RNA is operably linked to a nucleic acid sequence directing the expression of the guide RNA.

51. A vector comprising a nucleic acid of claim 49.

52. A cell comprising a guide RNA of claim 48.

53.-54. (canceled)

55. The cell of claim 52, further comprising an RNA-guided endonuclease, the activity of which is guided by the guide RNA.
